(19) United States
(12) Patent Application Publication
(10) Pub. No.: US 2011/0171291 A1
(43) Pub. Date: Jul. 14, 2011

(54) PATHOLOGICALLY-ACTIVATED THERAPEUTICS

(75) Inventor: Stuart A. Lipton, San Diego, CA (US)

(73) Assignee: Sanford-Burnham Medical Research Institute

(21) Appl. No.: 12/986,746

(22) Filed: Jan. 7, 2011

Related U.S. Application Data
(60) Provisional application No. 61/293,194, filed on Jan. 7, 2010.
(60) Provisional application No. 61/328,051, filed on Apr. 26, 2010.

(51) Int. Cl. A61K 9/127; G01N 33/53; C07K 16/00; G01N 33/573; C40B 30/04; A61K 39/395; A61P 25/28

(52) U.S. Cl. 424/450; 436/501; 530/387.1; 435/7.4; 506/9; 424/130.1

(57) ABSTRACT

Disclosed are methods and compositions for identifying, producing, and using pathologically-activated targeting compounds. Pathologically-activated compounds are compound that only have an effect, or have a disproportionate effect, on a target molecule when a pathological condition exists.

### CROSS-REFERENCE TO RELATED APPLICATIONS

This application claims benefit of U.S. Provisional Application No. 61/293,194, filed Jan. 7, 2010, and U.S. Provisional Application No. 61/328,051, filed Apr. 26, 2010. Application No. 61/293,194, filed Jan. 7, 2010, and Application No. 61/328,051, filed Apr. 26, 2010, are hereby incorporated herein by reference in their entirety.

### STATEMENT REGARDING FEDERALLY SPONSORED RESEARCH

This invention was made with government support under Grant Nos. P01 HD29587 and NIH EY05477 awarded by the National Institutes of Health (NIH). The government has certain rights in the invention.

### REFERENCE TO SEQUENCE LISTING

The Sequence Listing submitted Jan. 7, 2011 as a text file named “SBMRI_{—}12_{—}8403_AMD_AFD_Sequence_Listing.txt,” created on Jan. 6, 2011, and having a size of 2,006 bytes is hereby incorporated by reference pursuant to 37 C.F.R. §1.52(e)(5).

### FIELD OF THE INVENTION

The disclosed invention is generally in the field of therapeutics and treatment and specifically in the area of therapeutics affected by disease states.

### BACKGROUND OF THE INVENTION

The industrialized world faces an unusual predicament. The demographic shift towards an ageing population, coupled with the prevalence of Alzheimer's disease and vascular dementia (both of which cause cognitive decline), has led economists to estimate that by 2050, the entire economy of the industrialized world could be consumed by the costs of caring for the sick and elderly. What can be done to abate these dreaded neurodegenerative diseases and their socioeconomic effects?

Until recently, only symptomatic approaches were available—for example, cholinergic drugs for Alzheimer's disease, which mildly enhance memory by increasing levels of the neurotransmitter acetylcholine. But the concept of ‘brain protection’ raises hope that neurons can be preserved from the ravages of neurodegenerative insults.

In most neurodegenerative diseases, the brain is attacked and nerve cells are killed by a variety of overactive signaling pathways. These pathways are triggered by conditions such as oxidative or nitrosative stress, accumulation of aberrant proteins, and excessive activity in the brain of the neurotransmitter glutamate (excitotoxicity). Excitotoxic damage, a common final pathway contributing to most or all neurodegenerative disorders, is largely caused by overstimulation of N-methyl-D-aspartate (NMDA)-type glutamate receptors (NMDARs). This causes excessive influx of Ca²⁺ through the receptors' associated ion channels, resulting in detrimental enzymatic reactions and generation of toxic oxygen and nitrogen free radicals.

### BRIEF SUMMARY OF THE INVENTION

Many if not all drugs, particularly when developed against targets in the brain, manifest unacceptable clinical side effects. Described herein is a platform to discover pharmacological therapeutic agents that are pathologically activated, thereby avoiding side effects. The disclosed methods can use high-throughput screening (HTS) assays of chemical libraries combined in an iterative fashion with structural biology (crystallographic structural analysis with in silico screens). The ‘initial hits’ in this approach to drug discovery finds cryptic sites on drug targets that are exposed only upon excessive activation by agonist. Developing antagonists to these ‘cryptic’ sites insures that only excessive (pathological) activity of the drug target is curtailed by the drug thus developed, while sparing normal activity of the target.

The methods described herein can revolutionize the way that biopharmaceuticals are discovered. The methods are not merely new techniques for high-throughput screening (HTS) but rather a whole new way to look at how to discover new drug therapies.

The development of clinical therapeutic agents (drugs) with minimal side effects (to ensure clinical tolerability) is a big problem in the biopharmaceutical industry. Most drugs are currently discovered by high-affinity screening, resulting in drugs that strongly interact with their targets and block all functions, including any normal function of the target. The opposite approach has been taken in the disclosed methods in order to avoid side effects. Drugs need to be developed that only interact with the target when it is excessively (pathologically) activated and avoid the target under physiological conditions. These drugs can be referred to as Pathologically-Activated Therapeutics or “PAT” drugs because a pat is a gentle tap (Lipton, S. A. Nature Rev. Drug Discov. 5, 160-170 (2006); Lipton, S. A. Nature 428, 473 (2004); Lipton, S. A. et al. Cell Death Differ. 11, 18-20 (2004); Lipton S A. Nat. Rev Neurosci. 8, 803-808 (2007)); often, this means that the drug will be of relatively low affinity (binding only when the target becomes more available because of excessive/pathological activity). Specific pharmacological parameters should be achieved in order to produce such drugs, such as antagonists that are Uncompetitive with Fast Off-rates (which we recently termed UFO drugs) (Lipton S. A. Nat. Rev Neurosci. 8, 803-808 (2007)). This approach will be useful since the entire population would benefit from more clinically-tolerated drugs. The impact will be tremendous and can change the very roots of pharmaceutical drug development.

Examples of approaches to pathologically-activated therapeutics include UFO drugs, targeted S-nitrosylation, targeting of upregulated drug targets, target-induced drug activation, and activation of pro-drugs by abnormal redox states. For example, when a pathological condition exists the target molecule could be in a particular state to which the pathologically-activated compound differentially binds or is differentially targeted, whereupon the pathologically-activated compound has an effect on the target molecule. Thus, in this example, the target molecule is itself in a pathological state. The effect of the pathologically-activated compound can be, for example, via binding of the pathologically-activated compound to the target compound and/or via the pathologically-activated compound chemically reacting with the target molecule, another molecule associated with or near the target molecule, or a cell or tissue associated with or near the target molecule. For example, the pathologically-activated compound can be an inhibitor of the target molecule. As another example, the pathologically-activated compound can be linked to a nitro group that nitrosylates the target molecule, thus affecting the activity of the target molecule.

As another example, expression of the target molecule could be induced, upregulated or overexpressed under pathological conditions (such as in disease states) such that the pathologically-activated compound differentially accumulates where the induced, upregulated or overexpressed target molecule is induced, upregulated or overexpressed, whereupon the pathologically-activated compound has a differential effect on the target molecule due to the differential accumulation. Thus, in this example, the target molecule is not (necessarily) in a pathological state. Rather, the normal (or pathological) form of the target compound is upregulated or overexpressed. Compounds that bind to the target molecule but that have a high off-rate (and thus, relatively low affinity for the target compound) are useful forms of pathologically-activated compounds for this purpose. For example, the off-rate can be considered a fast off-rate if the off-rate has a ratio of 2:1 to the on-rate. In cases where the induced, upregulated or overexpressed target molecule can affect molecules that bind (such as enzymes that chemically alter substrate molecules), the pathologically-activated compound can be a pro-drug that can be activated by the target molecule. In such cases, the fact that the target molecule is differentially expressed under pathological conditions links the effect of the pathologically-activated compound to the pathological expression of the target molecule.

As another example, the pathologically-activated compound can be active only under particular conditions that occur under pathological conditions, whereupon the pathologically-activated compound affects the target molecule only under pathological conditions. For example, many pathological states are associated with oxidizing conditions. A compound that is differentially reactive or that is altered and activated under oxidizing conditions can be used as a pathologically-activated compound for pathological states that includes oxidizing conditions. For example, compounds are known that become electrophiles under oxidizing conditions. Such compounds can be used in or as pathologically-activated compounds.

Disclosed are methods and compositions for identifying, producing, and using pathologically-activated targeting compounds. Disclosed are methods of identifying a pathologically-activated targeting compound, the method comprising determining if a test compound binds uncompetitively to a target molecule, where if the test compound binds uncompetitively to the target molecule then the test compound is identified as a pathologically-activated targeting compound.

The disclosed methods can further comprise determining if the test compound is an uncompetitive inhibitor of the target molecule, where if the test compound is an uncompetitive inhibitor of the target molecule then the test compound is identified as a pathologically-activated compound. The disclosed methods can further comprise determining if the test compound has a fast off-rate for the target molecule. The disclosed methods can further comprise producing the pathologically-activated targeting compound. The disclosed methods can further comprise producing the pathologically-activated compound. The composition identified by any of the disclosed methods can be produced.

Also disclosed are methods of making a pathologically-activated compound, the method comprising producing a pathologically-activated compound identified by the disclosed methods. Also disclosed are methods of forming a pathologically-activated compound, the method comprising linking a nitro group to a pathologically-activated targeting compound. Also disclosed are methods of forming a pathologically-activated compound, the method comprising linking a redox-activated group to a targeting compound, where the redox-activated group becomes chemically reactive when exposed to oxidizing conditions or the redox-activated group becomes chemically reactive when exposed to reducing conditions. Also disclosed are pathologically-activated compounds, including pathologically-activated compounds identified, produced, or both by the disclosed methods.

The disclosed methods can be performed in various ways and using various conditions and components. For example, determining if a test compound binds uncompetitively to the target molecule and determining if the test compound is an uncompetitive inhibitor of the target molecule can be accomplished in different assays. As another example, determining if a test compound binds uncompetitively to the target molecule can be performed prior to determining if the test compound is an uncompetitive inhibitor of the target molecule. As another example, determining if the test compound is an uncompetitive inhibitor of the target molecule can be performed prior to determining if a test compound binds uncompetitively to the target molecule. As another example, determining if a test compound binds uncompetitively to the target molecule and determining if the test compound is an uncompetitive inhibitor of the target molecule can be accomplished in the same assay.

As another example, determining if the test compound binds uncompetitively to the target molecule can be accomplished by steps comprising bringing into contact the target molecule and the test compound and determining if the test compound exhibits uncompetitive binding to the target molecule. As another example, determining if the test compound is an uncompetitive inhibitor of the target molecule can be accomplished by steps comprising bringing into contact the target molecule and the test compound and determining if the test compound exhibits uncompetitive inhibition on the activity of the target molecule. As another example, determining if the test compound binds uncompetitively to the target molecule can be accomplished by comparing the binding of the test molecule in the presence of the same concentration of test compound and different concentrations of a molecule know to bind the target molecule. As another example, determining if the test compound is an uncompetitive inhibitor of the target molecule can be accomplished by comparing the activity of the target molecule in the presence of the same concentration of test compound and different concentrations of an agonist of the target molecule.

As another example, determining if the test compound binds uncompetitively to the target molecule can be determined by measuring the ratio of binding to the target molecule in the presence of a concentration of test compound and a first concentration of a molecule known to bind the target molecule compared to binding to the target molecule in the presence of the same concentration of test compound and a second concentration of the molecule known to bind the target molecule, where the first concentration of the molecule known to bind the target molecule is greater than the second concentration of the molecule known to bind the target molecule, where a ratio of greater than 1 indicates that the test compound binds uncompetitively to the target molecule. As another example, determining if the test compound is an uncompetitive inhibitor of the target molecule can be determined by measuring the ratio of the activity of the target molecule in the presence of a concentration of test compound and a first concentration of an agonist of the target molecule to the activity of the target molecule in the presence of the same concentration of test compound and a second concentration of an agonist of the target molecule, where the first concentration of agonist is greater than the second concentration of agonist, where a ratio of greater than 1 indicates that the test compound is an uncompetitive inhibitor of the target molecule.

As another example, determining if the test compound binds uncompetitively to the target molecule can be accomplished by comparing the binding of the test compound in the presence of the same concentration of test compound and different expression levels of the target molecule. As another example, determining if the test compound is an uncompetitive inhibitor of the target molecule can be accomplished by comparing the activity of the target molecule in the presence of the same concentration of test compound and different expression levels of the target molecule.

As another example, determining if the test compound binds uncompetitively to the target molecule can be determined by measuring the ratio of binding to the target molecule in the presence of a concentration of test compound and a first expression level of the target molecule compared to binding to the target molecule in the presence of the same concentration of test compound and a second expression level of the target molecule, where the first expression level of the target molecule is greater than the second expression level of the target molecule, where a ratio of greater than 1 indicates that the test compound binds uncompetitively to the target molecule. As another example, determining if the test compound is an uncompetitive inhibitor of the target molecule can be determined by measuring the ratio of the activity of the target molecule in the presence of a concentration of test compound and a first expression level of the target molecule to the activity of the target molecule in the presence of the same concentration of test compound and a second expression level of the target molecule, where the first expression level of the target molecule is greater than the second expression level of the target molecule, where a ratio of greater than 1 indicates that the test compound is an uncompetitive inhibitor of the target molecule.

As another example, the test compound can be a modified form of a prior test compound. The prior test compound can be, for example, determined to bind uncompetitively to the target molecule, determined to be an uncompetitive inhibitor of the target molecule, or both. The prior test compound can be a modified form of a second prior test compound. The second prior test compound can be, for example, determined to bind uncompetitively to the target molecule, determined to be an uncompetitive inhibitor of the target molecule, or both. The prior test compound can be produced via multiple iterations of modifying an earlier test compound that was determined to bind uncompetitively to the target molecule, determined to be an uncompetitive inhibitor of the target molecule, or both, and determining if the modified test compound binds uncompetitively to the target molecule, is an uncompetitive inhibitor of the target molecule, or both. Different iterations and/or different instances of determining if any of the test compounds binds uncompetitively to the target molecules and/or different iterations and/or different instances of determining if any of the test compounds is an uncompetitive inhibitor of the target molecule are accomplished with the same type of assays, different types of assays, or a combination.

As another example, a redox-activated group becomes chemically reactive when exposed to oxidizing conditions or other redox chemical insult, for example, pesticide-induced oxidative damage to neurons causing Parkinson's disease or oxidation from ischemic conditions leading to stroke or Vascular dementia. Thus, the redox-activated group can become chemically reactive when exposed to oxidizing conditions.

As another example, the test molecule can have an apparent affinity for the target molecule of about 100 nM or greater, an apparent affinity for the target molecule of about 1 μM or greater, an apparent affinity for the target molecule of about 10 μM or greater, or an apparent affinity for the target molecule of about 100 μM or greater. As another example, the target molecule can be a receptor, a neuroreceptor, a G-coupled receptor, a channel receptor, or an AMPA (α-amino-3-hydroxyl-5-methyl-4-isoxazole-propionate)-, kainate (KA)-, or N-methyl-D-aspartate (NMDA)-sensitive glutamate receptor.

As another example, determining if a test compound binds uncompetitively to a target molecule can be performed simultaneously on 96 or more (e.g., 384 or 1152) test compounds. As another example, determining if a test compound is an uncompetitive inhibitor of a target molecule can be performed simultaneously on 96 or more test compounds. As another example, determining if a test compound binds uncompetitively to a target molecule can be accomplished using a high throughput screen. As another example, determining if a test compound is an uncompetitive inhibitor of a target molecule can be accomplished using a high throughput screen.

Also disclosed are methods comprising administering to a subject in need of inhibition of an activated target molecule a composition comprising a pathologically-activated compound identified by the disclosed methods. Also disclosed are methods comprising administering to a subject in need of inhibition of a target molecule a composition comprising a pathologically-activated compound active only under conditions that occur in a pathological state. The conditions that occur in a pathological state can be oxidizing conditions. The pathologically-activated compound can become electrophilic under the conditions that occur in the pathological state. Also disclosed are methods comprising administering to a subject in need of inhibition of a pathologically-activated target molecule a composition comprising a compound identified by the disclosed methods.

Also disclosed are methods for identifying compounds that inhibit S-nitrosylation. For example, the method can be comprised of comparing S-nitrosylation of a target of interest in the presence and absence of a test compound, wherein an amount of S-nitrosylation of the target measured in the presence of the test compound that is less than the amount of S-nitrosylation of the target measured in the absence of the test compound identifies the test compound as an inhibitor of S-nitrosylation of that target. The amount of S-nitrosylation of the target can be measured by detecting conversion of 2,3-diaminonaphthalene (DAN) to 2,3-naphthyltriazole (NAT). Conversion of 2,3-diaminonaphthalene (DAN) to 2,3-naphthyltriazole (NAT) can be accomplished by measuring the fluorescence of NAT. This conversion is mediated by S-nitrosothiol formation due to donation of NO (nitric oxide species). In the context of these methods, the nitric oxide can be released from the S-nitrosylated targets. The nitric oxide can be released, for example, in the presence of by CuCl₂, suitable for high-throughput screening, although the assay was originally conceived of with HgCl₂ (but which is less suitable for high-throughput screening because mercury (Hg) can be quite toxic to the scientists performing the screening procedure). The target of interest can be any protein or molecule that can be S-nitrosylated (which would affect the protein's activity). Of most interest are proteins and other molecules that are aberrantly S-nitrosylated and/or are S-nitrosylated under pathological conditions. Useful targets of interest can be, for example, protein disulfide isomerase (PDI), dynamin-related protein 1 (Drp1), parkin, DJ-1, a MEF2 transcription factor, a matrix metalloproteinase (MMP), GAPDH, COX-2, or a peroxiredoxin (Prx) such as PrxII (McKercher et al., In: Encyclopedia of Life Sciences: Cell Death. Melino G, Vaux D, eds, John Wiley & Sons, 2010, pp. 262-272).

The subject can be suffering from or at risk for a disease characterized by an activated target molecule. The activated target molecule can have increased activity relative to a target molecule that is not activated. The activated target molecule can be a target molecule that is in a different state or has different potential relative to a target molecule that is not activated. The activated target molecule can be a target molecule expressed at a higher level than a normal or comparison level. In some forms, each copy of the target molecule expressed at a higher level than a normal or comparison level individually can have activity substantially the same as a copy of the target molecule expressed at the normal or comparison level, where the collective activity of the target molecule expressed at a higher level than a normal or comparison level is greater than the collective activity of the target molecule expressed at the normal or comparison level.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1a-1i show S-nitrosylation of protein disulfide isomerase (PDI) in vitro and in vivo. a, Recombinant PDI protein was incubated with SNOC(S-nitrosocysteine [a physiological NO donor capable of S-nitrosylating cysteine thiol groups], 100 μM) for 30 min at room temperature 21° C. S-nitrosylated PDI (SNO-PDI) thus generated was assessed by DAN assay (n=4 experiments). Open symbols, with SNOC; filled symbol, without SNOC. Values are mean±s.e.m. b, Cell lysates transduced with wild-type (WT) or mutant (N-terminal (Nmut), C-terminal (Cmut), or N-terminal and C-terminal (N&C)) PDI were immunoprecipitated with anti-PDI antibody. Immunoprecipitates were then incubated in the presence or absence of SNOC and subjected to DAN assay. Values are mean±s.e.m., n=5; asterisks, P<0.01 by analysis of variance. c, Top: cell lysates from human 293T cells were incubated with SNOC at room temperature to assay for SNO-PDI. Control samples were subjected to decayed (old) SNOC. SNO-PDI was detected by biotin-switch assay 30 min after SNOC exposure. Bottom: total PDI in cell lysates by western analysis. d-f, Top panels: Human embryonic kidney (HEK)-293 cells stably expressing neuronal nitric oxide synthase (nNOS) were assayed for endogenous SNO-PDI. nNOS was activated by Ca²⁺ ionophore A23187 (5 μM) in the presence or absence of NOS inhibitor (N-nitro-L-arginine; NNA). Bottom panels: Total PDI. Activation of nNOS increased endogenous SNO-PDI (d). NNA prevented this increase (e). Mutation of critical cysteine thiol groups of PDI also prevented its S-nitrosylation (f). g, SNO-PDI increased in cells exposed to rotenone in a NOS-dependent manner. Top: lysates from SH-SY5Y (neuronal-like) cells exposed to rotenone for 6 h in the presence or absence of NNA. Bottom: Total PDI. h, i, Brain tissues from controls, from PD patients with diffuse Lewy body disease (h), or AD patients (i) were subjected to biotin-switch assay to detect in vivo S-nitrosylation.

FIGS. 2a-2e show S-nitrosylation of PDI regulates its enzymatic activity. a, The effect of S-nitrosylation on PDI chaperone activity is shown. Unfolded rhodanese was diluted in buffer containing wild-type PDI (filled in circles), SNO-PDI (filled in squares), SNOC (filled in triangles), dominant-negative PDI (N-terminal and C-terminal mutant; open squares) or buffer alone (open circles). Rhodanese aggregation was monitored (n=8 experiments). Values are mean±s.e.m. b, S-nitrosylation of PDI attenuates its isomerase activity. Scrambled RNase A from bovine pancreas was incubated with wild-type (wt) PDI, dominant-negative (dn) PDI or SNO-PDI. Asterisks, P<0.01 for n=5 experiments. Values are mean±s.e.m. c, PDI inhibits the aggregation of synphilin-1. SH-SY5Y cells were transfected with green fluorescent protein (GFP)-synphilin-1 (GFP-Synp) and wild-type or dominant-negative PDI. Inclusion body formation was monitored by deconvolution microscopy 24 h after exposure to SNOC or control solution. Images were deconvolved with SlideBook software (Intelligent Imaging Innovations, Inc.). d, Percentage of cells with GFP-Synp inclusions. Values are mean±s.e.m. for n=2,500 transfectants counted in five experiments; asterisks, P<0.05. e, Co-localization of synphilin-1 and polyubiquitin (polyUb) by immunofluorescence (n=3).

FIGS. 3a-3d show that NMDA stimulates the accumulation of polyubiquitinated proteins and UPR pathway. a, SNO-PDI was detected in a NOS-sensitive manner in primary cortical cultures exposed to NMDA. b, Primary cortical cells infected with adenoviral Ad-LacZ or Ad-wtPDI were exposed to NMDA and immunostained for polyubiquitinated protein (indicated by grey arrows) and neuronspecific MAP2 (edges of grey circles at 0 h and grey streaks at 0 h and 12 h). Hoechst-stained DNA (inner part of grey circles at Oh, darker grey circles at 12 h, and darker grey circle at 24 h) was used to assess condensed, apoptotic nuclei (indicated by white arrow in merged image). c, Quantification of apoptotic and/or polyubiquitinated neurons similar to those shown in b. Left, apoptotic cells; middle, polyubiquitinated cells; right, apoptotic and polyubiquitinated cells. Values are ratio (×100%) of affected to total neurons, expressed as mean±s.e.m. for n=7,000 neurons counted in six experiments; asterisks, P<0.01. Open circles, Ad-LacZ; circles, Ad-wtPDI; squares, Ad-dnPDI; triangles, Ad-wtPDI plus N-nitro-L-arginine. d, NMDA-stimulated processing of XBP-1 mRNA and induction of the transcription factor CHOP. Processing of endogenous XBP-1 mRNA was evaluated by PstI endonuclease digestion of XBP-1 cDNA. TG, thapsigargin; bp, base pairs.

FIGS. 4a-4e show the neuroprotection by PDI against ER stress, proteasome inhibition, or Pael receptor expression. a-d, SH-SY5Y cells were transduced for 24 h with expression vectors for control lacZ (a-c), PDI constructs (a-d), or Pael receptor (d). Cultures were then incubated for 15 h in the presence or absence of 100 μM SNOC with 5 μM thapsigargin (a), 10 μgml⁻¹ tunicamycin (b) or 0.1 μM proteasome inhibitor MG132 (c). Exposure to SNOC abolished the protective effect of PDI on cell death induced by ER stress, proteasome inhibitor or Pael receptor. dn, dominantnegative; wt, wild type. For each panel, values are mean±s.e.m. for n=3,500 cells counted in five experiments; asterisks, P<0.01. e, Possible mechanism of SNO-PDI contributing to the accumulation of aberrant proteins and to cell death in human neurodegenerative disorders. UPS, ubiquitin-proteasome system.

FIG. 5 shows uncompetitive, pathologically activated therapeutic drugs. The target molecule harbors a cryptic allosteric site that only becomes exposed to the antagonist on pathological activation of the target, such as by oxidative stress or by an excessive agonist if the target is a receptor. An uncompetitive antagonist can then bind to the site, inhibiting the activity of the target molecule and bringing it back towards normal levels. When the pathological insult is removed, the binding site on the target becomes hidden again. Note that a relatively fast off-rate is often important for drug action in this case so that the antagonistic effect is not too prolonged.

FIG. 6 shows the inhibition (%) of competitive (descending line), uncompetitive (ascending line), and non-competitive (flat line) antagonists. An uncompetitive antagonist is a defined as a drug whose inhibitory action is contingent upon prior activation of the receptor by an agonist. For example, a fixed low micromolar dose of memantine blocks the effect of increasing concentrations of N-methyl-D-aspartate (NMDA) to a greater degree than it blocks lower concentration of NMDA. This is characteristic of an uncompetitive antagonist. By contrast, a fixed dose of a competitive antagonist will manifest less effect as the concentration of agonist increases because the agonist will outcompete the antagonist, rendering it less effective. A third type of antagonist is a noncompetitive antagonist. Here, the inhibitor binds to an allosteric site whose availability is not affected by the concentration of agonist. A noncompetitive antagonist is therefore equally effective at various concentrations.

FIGS. 7a and 7b show uncompetitive antagonism by memantine. a, Shows the degree of blockade of N-methyl-D-aspartate (NMDA)-induced current by 6 μM memantine (MEM) increases with rising concentration of NMDA. Transformation of this plot reveals that the value of the response ratio minus 1 (Λ−1) increases with rising NMDA concentrations, which is consistent with a mechanism of uncompetitive antagonism (right panel). The response ratio, Λ, is defined as the amplitude of NMDA-induced current in the absence of MEM divided by the amplitude of this current in the presence of MEM. b, Whole-cell current response induced by 20 and 50 μM NMDA, as recorded with a patch electrode at −60 mV, illustrates that the relative degree of blockade by MEM is greater for the higher concentration of agonist (NMDA). The degree of current blockade was calculated from the ratio of the steady-state current amplitude during MEM application relative to the steady-state current induced by NMDA alone just before MEM application (Chen, H.-S. V. et al. J. Neurosci. 12, 4427-4436 (1992)).

FIG. 8 shows a flowchart of an example of workflow for screening for “UFO” antagonists to the AMPA/KA receptor (GluRs).

FIG. 9 shows the targeted delivery of a pathologically-activated therapeutic drug. Specifically, this figure shows a nitro group (—NO₂⁻) that forms an adduct with memantine to form NitroMemantine, yielding two sites of action to antagonize NMDA receptors. One site of action of NitroMemantine, at which memantine acts, is in the ion channel, and a second site is on the extracellular surface of the receptor where the nitro group reacts with a cysteine thiol (—SH) group. Memantine binding to NMDA receptor-operated open channels has the effect of targeting the nitro group to the receptor. Note that NR1 and NR2A are subunits of the NMDA receptor. Glycine and NMDA (or glutamate) serve as co-agonists of the receptor. Both must be present for the channel to be activated and open (Lipton, S. A. Paradigm Nature Rev. Drug Discov. 5, 160-170 (2006)).

FIG. 10 shows the activation of a pathologically-activated therapeutic (PAT) drug by its target. The drug is not active until it interacts with its target, for example, an MMP (matrix metalloproteinase) enzyme. MMPs can digest extracellular matrix, and excess activity of these enzymes can contribute to neuronal cell death and other types of cell injury. The MMP inhibitor drug (or pro-drug) shown here is then increasingly activated upon binding to the catalytic site of the enzyme. Hence, increased or pathological activity of the enzyme results in further activation of the drug that in turn inhibits enzyme activity, resulting in blockade of MMP action in a feed-back type manner.

FIG. 11 shows the regulation of protein activity by electrophilic pro-drugs. Electrophiles have electron-deficient carbon centers and can alkylate cysteine thiols. Specifically, the figure shows a terpenoid with a catechol ring as an example of a pro-drug that becomes an electrophile (wavy line represents extended terpenoid structure that is not shown). The catechol is converted to a quinone-type electrophile by oxidation. A nucleophile is a chemical species that is electron-rich and seeks out an electron-deficient site for reaction. Subsequent nucleophilic attack of the electrophilic carbon (indicated by the arrow from the “protein target”) by a specific cysteine residue of a target protein (R—SH) modulates the protein's biological function. Through this type of reaction scheme, electrophilic compounds induce the transcription of many phase 2 enzymes. These phase 2 enzymes, such as heme oxygenase 1 (HO1; also known as HMOX1), are involved in drug detoxification, redox regulation and neuroprotection (Satoh, T. et al. Proc. Natl. Acad. Sci. USA 103, 768-773 (2006); Satoh, T. et al. Trends Neurosci 30, 37-45 (2007)). For a detailed discussion of the chemical reactions involved see (Satoh, T. et al. Trends Neurosci 30, 37-45 (2007)).

FIG. 12 shows a schema of the apoptotic-like cell injury and death pathways triggered by excessive NMDA receptor (NMDAR) activity. The cascade includes (a) NMDAR hyperactivation; (b) activation of the p38 mitogen-activated kinase (MAPK)-MEF2C (transcription factor) pathway (MEF2 is subsequently cleaved by caspases to form an endogenous dominant-interfering form that contributes to neuronal cell death); (c) toxic effects of free radicals such as nitric oxide (NO) and reactive oxygen species (ROS); and (d) activation of apoptosis-inducing enzymes including caspases and apoptosis inducing factor. AIF, apoptosis-inducing factor; Cyt c, cytochrome c; nNOS: neuronal nitric oxide synthase.

FIG. 13 shows NMDAR model illustrating important binding and modulatory sites. Glycine (Gly) and glutamate/N-methyl-D-aspartate (Glu/NMDA) are shown bound to their respective binding sites, and the binding site for Mg²⁺, MK-801 and memantine is within the ion channel pore region (Chen, H.-S. et al. J. Physiol. (Lond.) 499, 27-46 (1997); Chen, H.-S. et al. J. Neurosci. 12, 4427-4436 (1992); Stern-Bach, Y., et al. Neuron 13, 1345-1357 (1994); Monyer, H. et al. Science 256, 1217-1221 (1992); Nakanishi, S. Science 258, 597-603 (1992); Meguro, H. et al. Nature 357, 70-74 (1992); Kleckner, N. et al. Science 241, 835-837 (1988); Mayer, M. Nature 309, 261-263 (1984); Nowak, L. et al. Nature 307, 462-465 (1984); Mori, H. et al. Nature 358, 673-675 (1992); Huettner, J. Proc. Natl. Acad. Sci. USA 85, 1307-1311 (1988); Karschin, A., et al. J. Neurosci. 8, 2895-2906 (1988)). The Zn²⁺-binding site is also highlighted. The location of the cysteine sulfhydryl group (—SH) that reacts with nitric oxide (NO) species is labeled SNO. NR1, NMDA receptor subunit 1; NR2, NMDA receptor subunit 2A. Adapted from Lipton, S. A. Cell Death Differ. 11, 18-20 (2004).

FIG. 14 shows the chemical structure of memantine. Memantine has several important features: first, the three-ring structure; second, the bridgehead amine (—NH₂ group), which is charged at the physiological pH of the body (—NH₃⁺) and represents the region of memantine that binds at or near the Mg²⁺ binding site in the NMDAR-associated ion channel; and third, the methyl group (—CH₃) side chains (unlike amantadine), which serve to stabilize memantine's interaction in the channel region of the NMDAR.

FIG. 15 shows blockade of NMDA-evoked current by memantine and the relatively fast off-rate of a pathologically-activated therapeutic drug. Specifically, this figure shows a whole-cell recording of an NMDA-evoked current from a solitary neuron obtained with a patch electrode. The noisy current trace between 5 and 50 seconds indicates that at the resting potential, the on-time (that is, the time until peak blockade) of micromolar concentrations of memantine (MEM) is approximately 1 second. The off-time (that is, the time to recovery of the current after the washout of MEM) from the effect is approximately 5.5 seconds. The application of MEM produced an effective blockade only during NMDA receptor activation, which is consistent with the notion that its mechanism of action is open-channel blockade. That is, channels are only open in the presence of agonist NMDA, so MEM can only block the channels in the presence of the agonist. By contrast, MK-801 (also known as dizocilpine), a high-affinity NMDA receptor antagonist, would produce persistent blockade (top trace between 30 and 50 seconds) (Lipton, S. A. Paradigm Nature Rev. Drug Discov. 5, 160-170 (2006)). By contrast, MK-801 would produce persistent blockade (top trace after 30 seconds, simulated recording). See Chen, H.-S. V. et al. J. Neurosci. 12, 4427-4436 (1992). At a holding potential of approximately −50 mV, whole cell recording of NMDA-evoked current from a solitary neuron revealed that the on-time (time until peak blockade) of micromolar memantine (MEM) was approximately 1 second, whereas the off-time (recovery time) from the effect was ˜5.5 seconds (wide trace).

FIG. 16 shows the uncompetitive inhibition by memantine. Paradoxically, a fixed dose of memantine (e.g., 1 μM) blocks the effect of increasing concentrations of NMDA to a greater degree than lower concentrations of NMDA. This finding is characteristic of an uncompetitive antagonist, adapted from Chen, H.-S. V. et al. J. Neurosci. 12, 4427-4436 (1992).

FIGS. 17a and 17b show the effect of memantine on single-channel recordings of NMDAR-associated channels. Memantine blocks single-channel activity in outside-out patches from central neurons. Single-channel activity elicited in the same multi-channel patch by 100 μM NMDA (left) and by simultaneous application of 100 μM NMDA plus 12 μM memantine (right) at a holding potential of −60 mV. Filtered at 5 kHz; 50 μs digitization rate. See Chen, H.-S. V. et al. J. Neurosci. 12, 4427-4436 (1992).

FIGS. 18a and 18b show the relative lack of effect of memantine on the NMDA receptor component of excitatory postsynaptic currents (EPSCs). Compared with 1 μM MK-810, a similar concentration of memantine blocked little of the NMDA receptor-mediated component of the EPSC, which was recorded from the hippocampal autapse preparation. Note that the initial component of the EPSC is mediated by AMPA-sensitive glutamate receptors, whereas a delayed component is mediated by NMDA receptors. MK-801 produced use-dependent accumulation in the ion channels, leading to complete blockade of the NMDA receptor component, while memantine blocked only 10-15% of the NMDA receptor-mediated component of the EPSC under these conditions. See Chen, H.-S. V. et al. Neuroscience 86, 1121-1132 (1998).

FIGS. 19a and 19b show the fluorescence response indicating calcium ion entry induced by Kainate (KA) at AMPA-type glutamate receptor channels in cultured neurons. Fluorescent dye loading is accomplished by adding Fluo-4 NW to cells at 37° C. for 20 min and at RT (room temperature) for 10 min. The cells are incubated with compounds to be screened (generally 1 to 10 μM in 1% DMSO) for 2 min, followed by incubation with 200 μM kainate (KA) for 10 min (20 μM KA is used for the second, lower concentration of agonist—not shown here). Fluo-4 NW is a single-wavelength emitter which does not need to be washed out (no wash=NW); its fluorescence level can be readily quantified. FIG. 19 shows the relative fluorescence of cultured neurons treated with or without KA. FIG. 19a shows the value at 10 min after KA addition, while FIG. 19b plots the time course with the KA incubation. The Z-factor, a statistical indication of the robustness of a high-throughput screen, fluctuated between 0.54-0.73 through the time course. This range of Z-factors is more than adequate for HTS (a value of Z≧0.5 generally indicates a robust screen).

FIG. 20 shows a FLIRP Membrane Potential assay using inducible GluR1/GluR2 (unedited R form) AMPA receptors in an HEK293 cell line. After loading with membrane Potential FLIPR dye (Molecular Devices) for 1 hour at room temperature, membrane potential was monitored on a FlexStation 3 (Molecular Devices). The assay was run in the presence 100 μM cyclothiazide (CTZ, to minimize receptor desensitization when using AMPA or glutamate as the agonist to activate the receptors). Baseline fluorescence signal was read for 1 min, following which glutamate was added to reach a final concentration of 200 μM. Fluorescence intensity change was read for the ensuing 4 minutes. The Z-score calculated at peak response was 0.7 (n=11 samples measured for each condition).

FIGS. 21a and 21b show S-nitrosylation of PDI. a, SNOC but not diethylamine-NO (DEA-NO, an NO donor that does not readily nitrosylate cysteine thiol as shown in Uehara, T. et al. Nature 441, 513-517 (2006)) or hydrogen peroxide elicits S-nitrosylation of PDI. SH-SY5Y cells were incubated with DEA-NO, hydrogen peroxide (H₂O₂), or SNOC for 30 min. (Top) Cell lysates were subjected to the biotin-switch assay to assess S-nitrosylation of PDI (SNO-PDI). (Bottom) Total PDI in cell lysates. b, HEK293 cells stably expressing nNOS were treated with A23187 (to increase intracellular calcium ions and thus activate nNOS to generate endogenous NO), and then PDI S-nitrosylation was assessed by using the biotin-switch assay in the presence or absence of ascorbate (Asc). Input: total PDI in cell lysates.

FIG. 22 shows the LC-MS/MS (Liquid chromatography-tandem mass spectrometry) analysis of PDI showing modified cysteine thiol groups in the C-terminal CGHC motif. Deconvoluted MS/MS spectra of precursor ion at m/z 939.41²⁺ (inset) obtained from a tryptic digest of PDI after exposure to as little as 10 μM SNOC in vitro. The m/z 939.41²⁺ precursor ion corresponds to the PDI peptide ³⁷⁰NVFVEFYAPWCGHCK³⁸⁴ (SEQ ID NO:1) with sulfination (SO₂H) and MMTS (SSCH₃) modifications at the two cysteine (Cys) residues, respectively. Analysis of the structurally informative fragment ions, y₄, y₇, and b₁₀ in multiple spectra confirmed the identity of the C-terminal peptide containing the sulphinated ³⁸⁰Cys residue. Sulphination was only observed after exposure to an NO donor. A, alanine; C, cysteine; E, glutamate; F, phenylalanine; G, glycine; H, histidine; K, lysine; N, asparagines; P, proline; V, valine; W, tryptophan; Y, tyrosine.

FIGS. 23a and 23b show S-nitrosylation of PDI in human brains. a, (Top) Brain tissues from Alzheimer's disease (AD) patients were subjected to the biotin-switch assay to assess in vivo S-nitrosylation. (Bottom) Total PDI in brain lysates by Western blot. b, Ratio of increased SNO-PDI formation. Blots from the biotin-switch assay and Western analyses were quantified by densitometry, and the relative ratio of SNO-PDI to total PDI calculated. Subsequently, the SNO-PDI ratio comparing patient brains to control brains or NO donor-exposed cells to control cells was determined. Values are mean±s.e.m., *P<0.01 by ANOVA.

FIG. 24 shows the effect of oxidative and nitrosative stress on PDI activity. Chaperone activity of PDI was assessed by standard methods measuring the degree of aggregation of rhodanese as described elsewhere herein. Unfolded rhodanese was incubated with wild-type (wt)PDI, SNOC-exposed PDI (SNO-PDI), hydrogen peroxide-exposed PDI (H₂O₂—PDI), or buffer alone (Spontaneous). Values are mean±s.e.m., n=3; *P<0.01 by ANOVA.

FIG. 25 shows the colocalization of overexpressed PDI protein with the endoplasmic reticulum (ER) marker Calreticulin in SH-SY5Y cells. Cells were transfected with wild-type PDI (pCR-wtPDI) using Lipofectamine2000. Immunostaining with anti-PDI antibody revealed high-intensity staining of PDI (grey areas in PDI images and identical areas in the merged images) compared to endogenous PDI staining in control cells. Double immunostaining revealed that overexpressed PDI (grey areas in PDI images and identical areas in the merged images) was exclusively colocalized with the ER marker Calreticulin (grey areas in Calreticulin images and identical areas in the merged images) in a perinuclear pattern (white arrow merged image). Cell nuclei were counterstained with Hoechst dye (grey areas in Hoechst images and identical areas in the merged images).

FIGS. 26a and 26b show the effect of endogenous NO and transduced PDI on cell survival. a, Effect of NOS inhibition and PDI expression on NMDA-induced damage of cerebrocortical neurons. Excitotoxic injury was monitored by the extent of dendritic damage, visualized with MAP2 staining NMDA-induced neuronal injury was attenuated by the NOS inhibitor L-Nitroarginine (NNA) and by transduction with adenoviral PDI. *P<0.01 by ANOVA compared to lacZ infection and NMDA. b, Effects of thapsigargin, tunicamycin, and MG132 on SH-SY5Y cell death. Cells were treated with an ER stressor (thapsigargin or tunicamycin) or a proteasome inhibitor (MG132), and stained with Hoechst 33342 to assess apoptotic nuclei. As a control, cells were also transduced with adenoviral constructs to verify that infection with PDI did not affect cell survival by itself *P<0.01 by ANOVA compared to lacZ infection alone.

FIGS. 27a and 27b show the overexpression of PDI in cerebrocortical neurons by adenoviral (Ad) infection. a, PDI expression in lysates of cells infected by Ad-wtPDI at various multiplicities of infection (MOI). Primary cortical cultures were infected by Ad-wtPDI at 2.5, 5, 10 or 20 MOI, and incubated for 48 h. Then, total cell lysates were prepared and subjected to Western blot analysis with anti-PDI antibody. PDI expression significantly increased after each infection and reached a plateau at a MOI of 10. b, Primary cerebrocortical cultures of neurons were infected with Ad-lacZ, Ad-wtPDI or Ad-dnPDI at a MOI of 10 for 6 h, rinsed, and then further incubated for 48 h. The cells were fixed, washed with phosphate-buffered saline (PBS), and immunostained with anti-microtubule associated protein-2 (MAP2, a specific marker for neurons) polyclonal Ab (pAb, grey areas in MAP2 images and identical areas in the merged images) and anti-PDI monoclonal antibody (mAb, grey areas in PDI images and identical areas in the merged images). Primary antibodies (1:200 dilution) were incubated for 12 h at 4° C. After washing, the cells were incubated for 1 h at 37° C. with anti-mouse and anti-rabbit Abs conjugated with Alexa-488 and -594 (1:200 dilution), respectively, and then incubated in Hoechst 33342 dye to fluorescently stain nuclei and allow assessment of nuclear morphology for the presence of apoptosis. Fluorescent images revealed high levels of expression of the PDI constructs in neurons with the brightest staining pattern localized to perinuclear and peripheral structures, consistent with ER, as demonstrated previously (Tanaka, S., et al. J. Biol. Chem., 275, 10388-10393, (2000); Conn, K. J., et al. Brain Res., 1022, 164-172, (2004)).

FIG. 28 shows a FLIPR assay using Calcium 5 FLIPR kit on HEK293 cells stably transduced with the GluR2Q subunit of the AMPA receptor. After loading with Calcium 5 for 1 hour at 37° C., [Ca²⁺]ᵢ was monitored on a FlexStation 3 (Molecular Devices). The assay was run in the presence of 100 μM cyclothiazide (CTZ, which blocks desensitization of GluR2 homomeric channels responding to AMPA as the agonist) and 10 mM Ca²⁺ to increase the signal in response to AMPA. The baseline fluorescence signal was read for 1 minute, then 200 μM AMPA was added. Fluorescence intensity change was read for the next 4 minutes. As a control, NBQX (a known AMPA receptor antagonist) was pre-added into each well at a concentration of 100 μM. The Z-score calculated at the peak of the calcium signal for 200 μM AMPA vs. blank buffer experiment was 0.8 (n=10 for each condition).

### DETAILED DESCRIPTION OF THE INVENTION

The disclosed method and compositions may be understood more readily by reference to the following detailed description of particular embodiments and the Example included therein, and to the Figures and their previous and following description.

Disclosed are methods and compositions for identifying, producing, and using pathologically-activated targeting compounds. Disclosed are methods of identifying a pathologically-activated targeting compound, the method comprising determining if a test compound binds uncompetitively to a target molecule, where if the test compound binds uncompetitively to the target molecule then the test compound is identified as a pathologically-activated targeting compound.

The development of neuroprotective drugs is essential for the treatment or management of various neurological disorders. These disorders range from acute stroke and head or spinal-cord trauma to more chronic neurodegenerative diseases such as Alzheimer's disease, cognitive dysfunction in Down syndrome, Parkinson's disease, Huntington's disease, amyotrophic lateral sclerosis (ALS), multiple sclerosis, HIV-associated dementia or HIV-associated neurocognitive disorder, and glaucoma. Additionally, cardiac diseases, digestive diseases, and other systemic maladies are included. However, clinical use of neuroprotective treatments has been limited owing to serious side effects. These include cognitive problems, hallucinations and even coma (Lipton, S. A., Nature Rev. Drug Discov. 5, 160-170 (2006); Koroshetz, W. et al. Trends Pharmacol. Sci. 17, 227-233 (1996); Kemp, J. et al. Nature Neurosci. 5, 1039-1042 (2002), all of which can occur as a result of interference of the drugs with normal brain function. Indeed, because the brain is a complex organ that is capable of many intricate functions, it has proved difficult to develop drugs for the treatment of neurodegenerative disorders that do not interfere with the normal functioning of the nervous system. Although many factors, including absorption, distribution, metabolism, excretion (ADME) and pharmacokinetics, complicate drug development in general, brain function is particularly susceptible to disruption because many of the targets for drug action exert normal physiological actions in unaffected parts of the brain. Strong inhibition of these targets can block normal as well as abnormal activity. The same could be said for the rest of the body, but brain function appears to be particularly susceptible to side effects that cause clinical intolerability of drugs.

The neurodegenerative processes can be drug targets for neuroprotection. For example, injurious agents or instigators of degeneration could be counteracted, as in the case of excessive amounts of glutamate, which excite neurons to death, or aberrant proteins (such as mutant parkin protein in early-onset Parkinson's disease) that clog the brain's detoxification and degradation systems or otherwise disrupt normal function of the cell. Other examples include the depression of ‘death’ signaling pathways by inhibiting enzymes such as caspases; restoration of pathways that degrade aberrant proteins through the proteosomal and lysosomal systems; or enhancement of survival pathways, with either anti-cell-death proteins or neurotrophic factors that prolong neuronal life. Cell-based approaches could also be advanced with attempts to ameliorate inflammatory cell activities, and stem-cell replacement therapy to provide new brain cells.

However, many of these targets have normal functions, and the targets only become deleterious to the nervous system when in excess, when overactivated, or under maladaptive conditions. For example, physiological NMDA-type glutamate receptor activity is essential for normal neuronal development, communication between neurons, and memory formation. Neuroprotective agents that work by high-affinity binding to these receptors block all activity, and these drugs produce unacceptable side effects including hallucinations, drowsiness and coma. Similarly, microglia and astrocytes nurture and protect the neurons they surround, and only release toxic substances when they are inappropriately perturbed. These cell processes and molecules cannot simply be shut off without compromising normal or adaptive functions.

Targeting neuroprotective drugs and cell-based therapies at abnormal functions in an appropriate spatial and temporal pattern for the disease, while sparing normal, physiological activity are needed. However, drug discovery often involves high-affinity screening of target molecules. Thus, drugs can work “too well” and “all the time.” Analogous of the target molecule can be a television set, where “competitive” drugs battle one-on-one with the agonist at the on/off switch (the agonist binding site) and, when successful, will simply turn the set off. A neuroprotective agent that works by high-affinity binding to the receptor of a neurotransmitter will block all activity—normal and abnormal. In addition, such drugs will overpower lower (physiological) levels of neurotransmitter more effectively than higher (pathological) levels. Thus, normal brain areas will be shut off even before pathological areas are effectively protected. Thus, such drug use would result in unacceptable side effects by blocking normal physiological activity in all parts of the brain, even those not affected by the disease process.

Ideally, drugs should selectively bind under pathological conditions while not binding well under physiological conditions. For example, excitotoxicity could be prevented by turning down the excessive amounts of Ca²⁺ influx through the receptor's channel towards normal, thus avoiding the formation of free radicals. However, if the drug binds with high affinity in the channel, it will accumulate there, and block normal function.

Contrarily, a clinically tolerated drug would block only excessive activity while relatively sparing normal function, simply by adjusting the amounts of Ca²⁺ towards normal levels. Such drugs can be termed uncompetitive inhibitors and they work better when increasing levels of agonist are present, thereby blocking excessive (pathological) receptor activity while sparing lower (physiological) activity. This mechanism is not only drug selectivity for the target despite low affinity but, most importantly, give the drug the ability to come off the target relatively quickly, preventing accumulation and blocking of subsequent normal function. In example, memantine can preferentially block NMDA-glutamate receptor-associated ion channels when they are excessively open. Memantine is approved for treatment of Alzheimer's disease in Europe and the United States.

By virtue of their relatively gentle binding, drugs of this type work best under pathological conditions, while exerting minimal effects on normal brain activity. This simple concept can be extended to other neuroprotective targets or other pharmaceuticals.

The disclosed methods use these and other principles described herein to provide such pathologically-activated therapeutics, including methods for identifying, producing, testing, modifying, administering, and using pathologically-activated compounds.

Examples of approaches to pathologically-activated compounds and therapeutics include, for example, UFO drugs, targeted S-nitrosylation, targeting of upregulated drug targets, target-induced drug activation, and activation of pro-drugs by abnormal redox states. For example, when a pathological condition exists the target molecule could be in a particular state to which the pathologically-activated compound differentially binds or is differentially targeted, whereupon the pathologically-activated compound has an effect on the target molecule. Thus, in this example, the target molecule is itself in a pathological state. The effect of the pathologically-activated compound could be, for example, via binding of the pathologically-activated compound to the target compound and/or via the pathologically-activated compound chemically reacting with the target molecule, another molecule associated with or near the target molecule, or a cell or tissue associated with or near the target molecule. For example, the pathologically-activated compound can be an inhibitor of the target molecule. As another example, the pathologically-activated compound can be linked to a nitro group that nitrosylates the target molecule, thus affecting the activity of the target molecule.

As another example, expression of the target molecule could be induced, upregulated or overexpressed under pathological conditions (such as in disease states) such that the pathologically-activated compound differentially accumulates where the induced, upregulated or overexpressed target molecule is induced, upregulated or overexpressed, whereupon the pathologically-activated compound can have a differential effect on the target molecule due to the differential accumulation. Thus, in this example, the target molecule is not (necessarily) in a pathological state. Rather, the normal (or pathological) form of the target compound is upregulated or overexpressed. Compounds that bind to the target molecule but that have a high off-rate (and thus, relatively low affinity for the target compound) are useful forms of pathologically-activated compounds for this purpose. In cases where the induced, upregulated or overexpressed target molecule can affect molecules that bind (such as enzymes that chemically alter substrate molecules), the pathologically-activated compound can be a pro-drug that can be activated by the target molecule. In such cases, the fact that the target molecule is differentially expressed under pathological conditions links the effect of the pathologically-activated compound to the pathological expression of the target molecule.

As another example, the pathologically-activated compound can be active only under particular conditions that occur under pathological conditions, whereupon the pathologically-activated compound affects the target molecule (or its environment) only under pathological conditions. For example, many pathological states are associated with oxidizing conditions. A compound that is differentially reactive or that is altered and activated under oxidizing conditions can be used as a pathologically-activated compound for pathological states that includes oxidizing conditions. For example, compounds are known to become electrophiles under oxidizing conditions. Such compounds can be used in or as pathologically-activated compounds.

A “pathologically-activated compound” is a compound that only has an effect, or has a disproportionate effect, on a target molecule when a pathological condition exists. A “targeting compound” is a compound that binds to and/or accumulates at the site of a target molecule. A “pathologically-activated targeting compound” is a targeting compound that only binds to and/or accumulates at the site of a target molecule when a pathological condition exists. A pathologically-activated targeting compound can be a pathologically-activated compound. However, not all pathologically-activated compounds are pathologically-activated targeting compounds. Pathologically-activated targeting compounds are targeted to a target molecule when pathological conditions exist but may or may not affect the target compound. Pathologically-activated compounds affect the target molecule when pathological conditions exist but may or may not be targeted to the target molecule.

“An “activated target molecule” is a target molecule that has increased activity relative to a target molecule that is not activated or is a target molecule that is in a different state or has different potential relative to a target molecule that is not activated. A target molecule expressed at a higher level than a normal or comparison level can be an activated target molecule. A “disproportionate effect” refers to an effect that is 2, 3, 4, 5, 6, 7, 8, 9, 10, 20, 30, 40, 50, or 100 times greater in magnitude than a comparison effect.

“Test compound,” “test molecule,” and the like refer to any compounds, molecules, and the like that are to be used in the disclosed methods. For example, any compounds, molecules, and the like that are to be tested or screened in the disclosed methods for the desired or indicated activity. For example, compounds in chemical libraries can be used as test compounds.

“Identify,” identification,” and other forms refer to a selection of a compound, molecule, and the like, or a determination that a property, condition, characteristic, and the like exists, such as in a compound, molecule, and the like.

The term “hit” refers to a test compound that shows desired properties in an assay. The term “test compound” refers to a chemical to be tested by one or more screening method(s) as a putative modulator. A test compound can be any chemical, such as an inorganic chemical, an organic chemical, a protein, a peptide, a carbohydrate, a lipid, or a combination thereof. Usually, various predetermined concentrations of test compounds are used for screening, such as 0.01 micromolar, 1 micromolar and 10 micromolar. Test compound controls can include the measurement of a signal in the absence of the test compound or comparison to a compound known to modulate the target.

The term “modulate” as used herein refers to the ability of a compound to change an activity in some measurable way as compared to an appropriate control. As a result of the presence of compounds in the assays, activities can increase or decrease as compared to controls in the absence of these compounds. Preferably, an increase in activity is at least 25%, more preferably at least 50%, most preferably at least 100% compared to the level of activity in the absence of the compound. Similarly, a decrease in activity is preferably at least 25%, more preferably at least 50%, most preferably at least 100% compared to the level of activity in the absence of the compound. A compound that increases a known activity is an “agonist”. One that decreases, or prevents, a known activity is an “antagonist”.

The term “inhibit” means to reduce or decrease in activity or expression. This can be a complete inhibition of activity or expression, or a partial inhibition. Inhibition can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

The term “monitoring” as used herein refers to any method in the art by which an activity can be measured.

The term “providing” as used herein refers to any means of adding a compound or molecule to something known in the art. Examples of providing can include the use of pipettes, pipettemen, syringes, needles, tubing, guns, etc. This can be manual or automated. It can include transfection by any mean or any other means of providing nucleic acids to dishes, cells, tissue, cell-free systems and can be in vitro or in vivo.

The term “preventing” in the context of disease, disease conditions, and pathological conditions as used herein refers to administering a compound prior to the onset of clinical symptoms of a disease or conditions so as to prevent a physical manifestation of aberrations associated with the disease or condition.

By “treatment” and “treating” is meant the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, ameliorization, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

The term “in need of treatment” as used herein refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that include the knowledge that the subject is ill, or will be ill, as the result of a condition that is treatable by the disclosed compounds.

As used herein, “subject” includes, but is not limited to, animals, plants, bacteria, viruses, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

The terms “higher,” “increases,” “elevates,” or “elevation” refer to increases above basal levels, e.g., as compared to a control. The terms “low,” “lower,” “reduces,” or “reduction” refer to decreases below basal levels, e.g., as compared to a control.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### Methods

Disclosed are methods for identifying, producing, and using pathologically-activated targeting compounds. Disclosed are methods of identifying a pathologically-activated targeting compound, the method comprising determining if a test compound binds uncompetitively to a target molecule, where if the test compound binds uncompetitively to the target molecule then the test compound is identified as a pathologically-activated targeting compound. Also disclosed are methods comprising administering to a subject in need of inhibition of an activated target molecule a composition comprising a pathologically-activated compound identified by the disclosed methods. Also disclosed are methods comprising administering to a subject in need of inhibition of a target molecule a composition comprising a pathologically-activated compound active only under conditions that occur in a pathological state. The conditions that occur in a pathological state can be oxidizing conditions. The pathologically-activated compound can become electrophilic under the conditions that occur in the pathological state.

### A. Uncompetitive Antagonists

An uncompetitive drug is a receptor antagonist whose inhibitory action depends on prior activation of the receptor by an agonist (see FIG. 6). An example of such drug is the N-methyl-D-aspartate (NMDA) open channel clocker memantine (Chen, H.-S. V. and Lipton, S. A. J. Physiol. 499, 27-46 (1997); Chen, H.-S. V. et al. J. Neurosci. 12, 4427-4436 (1992)). Excessive activation of NMDA-type glutamate receptors (NMDARs) is thought to contribute to a wide range of neurological disorders including acute stroke, CNS trauma, Huntington's disease, Alzheimer's disease, Parkinson's disease, HIV-associated dementia, multiple sclerosis and glaucoma (Lipton, S. A. et al. N. Engl. J. Med. 330, 613-622 (1994); Choi, D. W. Neuron 1, 623-634; Meldrum, B. et al. Trends Pharmacol. Sci. 11, 379-387 (1990); Rothman, S. et al. Trends Neurosci. 18, 57-58 (1995); Mattson, M. Nature 430, 631-639 (2004); Lipton, S. A. Curr. Opin. Neurol. Neurosurg. 6, 588-596 (1993); Lipton, S. A. et al. N. Engl. J. Med. 332, 934-940 (1995); Kaul, M., et al. Nature 410, 988-994 (2001); Dreyer, E. B. JAMA 281, 306-308 (1999); De Felice, F. G. et al. J. Biol. Chem. 282, 11590-11601 (2007); Lacor, P. N. et al. J. Neurosci. 27, 796-807 (2007); Shankar, G. M. et al. J. Neurosci. 27, 2866-2875 (2007); Lipton, S. A. Neuron 50, 9-11 (2006)). Overactivation of NMDARs results in disproportionate opening of ion channels that are coupled to these receptors. Excessive opening of ion channels causes an exaggerated Ca²⁺ influx, which can result in the pathological activation of enzymes and the formation of free radicals (Lipton, S. A. Nature Rev. Drug Discov. 5, 160-170 (2006); Lipton, S. A. et al. N. Engl. J. Med. 330, 613-622 (1994)). The drug memantine blocks these ion channels preferentially when they are excessively open (hence, the term open-channel blocker). Indeed, memantine administration has been shown to result in the preferential blockade of excessive (usually extrasynaptic) NMDAR activity while sparing normal excitatory synaptic function (Lipton, S. A. Nature Rev. Drug Discov. 5, 160-170 (2006); Okamoto S.-i. et al., Nature Med. 15, 1407-1413 (2009)).

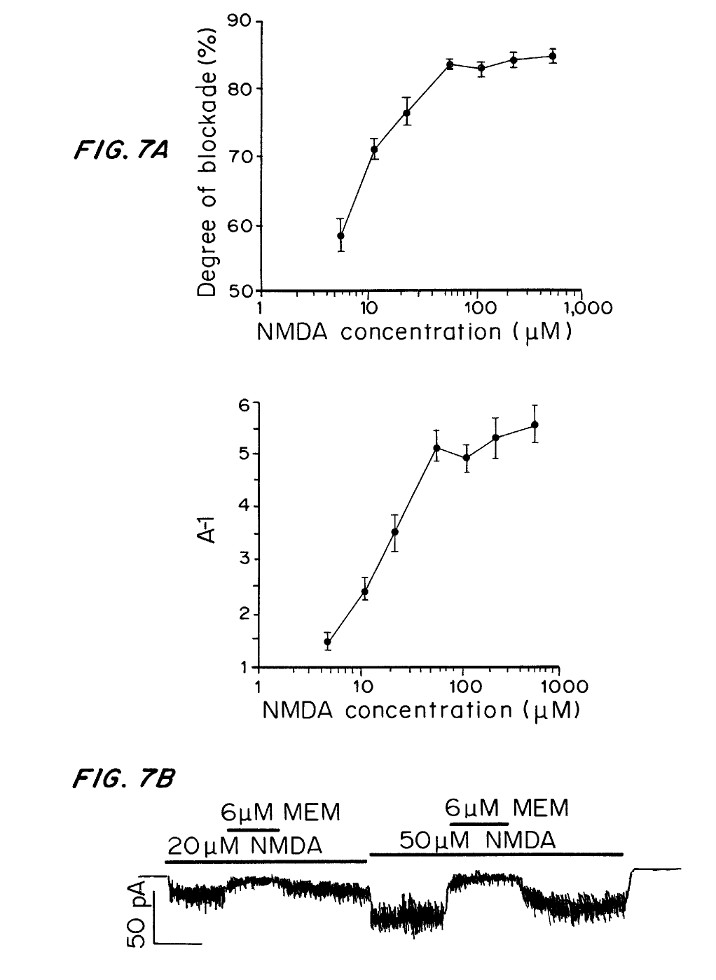

Paradoxically, a low micromolar concentration of memantine blocks the effects of higher concentrations of an NMDAR agonist to a greater degree than the effects of lower concentrations of agonist (see FIG. 7). This intriguing phenomenon can be explained by the fact that higher concentrations of agonist result in a greater fraction of open channels. As memantine blocks only open channels, relatively more channels are blocked when more agonist is present. Thus, memantine is an example of a drug that has an uncompetitive mechanism of action and blocks high, pathological levels of agonist more effectively than lower, physiological levels.

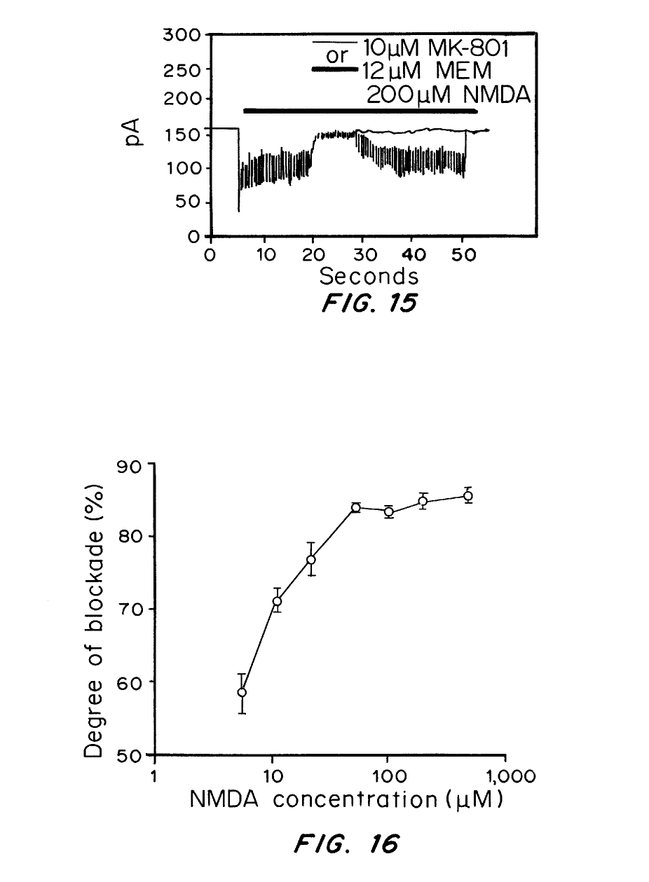

A feature that can greatly improve the clinical tolerability of an uncompetitive antagonist is a fast off-rate. This ensures that the drug does not, for example, accumulate in the ion channels and therefore does not block subsequent synaptic neurotransmission (Chen, H.-S. V. and Lipton, S. A., J. Neurochem. 97, 1611-1626 (2006); Okamoto S.-i. et al., Nature Med. 15, 1407-1413 (2009)). Drugs with an uncompetitive mechanism of action and a relatively fast off-rate can be referred to as UFO drugs. Many existing uncompetitive drugs do not have fast off-rates owing to their structure-receptor interactions. However, memantine has a relatively fast off-rate and is therefore an example of an UFO drug (see FIG. 15). Its fast off-rate contributes to its low affinity for the NMDAR (Lipton, S. A. Nature Rev. Drug Discov. 5, 160-170 (2006; Okamoto S.-i. et al., Nature Med. 15, 1407-1413 (2009)).

So, despite its low affinity, memantine is highly effective and selective for the NMDAR activity, notwithstanding its slight activity at 5-hydroxytryptamine 3 (5-HT3) and nicotinic α-7 receptors (Chen, H.-S. V. and Lipton, S. A., J. Neurochem. 97, 1611-1626 (2006); Okamoto S.-i. et al., Nature Med. 15, 1407-1413 (2009)). This indicates that effective and selective drugs do not need to have a high affinity. Most existing drugs have been discovered using high-affinity screening procedures, reflecting the assumption that a high affinity was thought to be required for selectivity. But high affinity does not necessarily guarantee high selectivity and vice versa. In fact, the methods described herein that screen for uncompetitive drugs against other targets allow for development of new forms of these types of drug.

An interesting analogy can be drawn between the action of memantine and the normal function of the NR3 subunit of the NMDAR, which is present predominantly during brain development (Das, S. et al. Nature 393, 377-381 (1998); Sucher, N. J. et al. J. Neurosci. 15, 6509-6520 (1995); Wong, H. K. et al. J. Comp. Neurol. 450, 303-317 (2002); Chatterton, J. E. et al. Nature 415, 793-798 (2002)). This subunit inhibits excessive Ca²⁺ influx and consequent neurotoxicity by blocking the outer channel vestibule of the NMDA-associated pore (Chen, H.-S. V. and Lipton, S. A., J. Neurochem. 97, 1611-1626 (2006); Wada, A., Takahashi, H., Lipton, S. A. & Chen, H.-S. V. NR3A modulates the outer vestibule of the “NMDA” receptor channel. J. Neurosci. 26, 13156-13166 (2006)). Thus, in some sense the action of memantine mimics that of this naturally occurring NMDAR subunit. Indeed, recent work in our laboratory has shown that this subunit provides neuroprotection during the neonatal period (Lipton, S. A. Nature Rev. Drug Discov. 5, 160-170 (2006); Nakanishi, N. et al., Neuroprotection by the NR3A subunit of the NMDA receptor. J Neurosci 2009; 29:5260-5265).

For uncompetitive binders, for example, when a pathological condition exists the target molecule could be in a particular state to which the ‘pathologically-activated compound’ differentially binds, whereupon the pathologically-activated compound has an effect on the target molecule. Thus, in this example, the target molecule is itself in a pathological state. An uncompetitively binding pathologically-activated compound can, for example, bind to abnormally activated or abnormally expressed target molecules. The effect of the pathologically-activated compound could be, for example, via binding of the pathologically-activated compound to the target compound and/or via the pathologically-activated compound chemically reacting with the target molecule, another molecule associated with or near the target molecule, or a cell or tissue associated with or near the target molecule. For example, the pathologically-activated compound can be an inhibitor of the target molecule.

Pathologically-activated targeting compounds useful for this form of the disclosed methods can be identified by determining if a test compound binds uncompetitively to a target molecule, where if the test compound binds uncompetitively to the target molecule then the test compound is identified as a pathologically-activated targeting compound. These methods can further comprise determining if the test compound is an uncompetitive inhibitor of the target molecule, where if the test compound is an uncompetitive inhibitor of the target molecule then the test compound is identified as a pathologically-activated compound. These methods can also further comprise determining if the test compound has a fast off-rate for the target molecule. These methods can also further comprise producing pathologically-activated compounds and pathologically-activated targeting compounds that bind uncompetitively to a target molecule.

Uncompetitive binding is a characteristic of the relationship of the binding of a compound, molecule, ligand, and the like to another molecule and its activity and/or binding to another compound, molecule, ligand, and the like, the principles of which are well known and features of which are described herein. A compound, molecule, ligand, and the like that exhibits uncompetitive binding can be said to bind uncompetitively to the other molecule. Uncompetitive inhibition is a characteristic of the relationship of the inhibition of a compound, molecule, ligand, and the like of the activity of another molecule, the principles of which are well known and features of which are described herein.

These forms of the methods can be performed in various ways and using various conditions and components. For example, determining if a test compound binds uncompetitively to the target molecule and determining if the test compound is an uncompetitive inhibitor of the target molecule can be accomplished in different assays. As another example, determining if a test compound binds uncompetitively to the target molecule can be performed prior to determining if the test compound is an uncompetitive inhibitor of the target molecule. As another example, determining if the test compound is an uncompetitive inhibitor of the target molecule can be performed prior to determining if a test compound binds uncompetitively to the target molecule. As another example, determining if a test compound binds uncompetitively to the target molecule and determining if the test compound is an uncompetitive inhibitor of the target molecule can be accomplished in the same assay.

As another example, determining if the test compound binds uncompetitively to the target molecule can be accomplished by steps comprising bringing into contact the target molecule and the test compound and determining if the test compound exhibits uncompetitive binding to the target molecule. As another example, determining if the test compound is an uncompetitive inhibitor of the target molecule can be accomplished by steps comprising bringing into contact the target molecule and the test compound and determining if the test compound exhibits uncompetitive inhibition on the activity of the target molecule. As another example, determining if the test compound binds uncompetitively to the target molecule can be accomplished by comparing the binding of the test molecule in the presence of the same concentration of test compound and different concentrations of a molecule know to bind the target molecule. As another example, determining if the test compound is an uncompetitive inhibitor of the target molecule can be accomplished by comparing the activity of the target molecule in the presence of the same concentration of test compound and different concentrations of an agonist of the target molecule.

As another example, determining if the test compound binds uncompetitively to the target molecule can be determined by measuring the ratio of binding to the target molecule in the presence of a concentration of test compound and a first concentration of a molecule known to bind the target molecule compared to binding to the target molecule in the presence of the same concentration of test compound and a second concentration of the molecule known to bind the target molecule, where the first concentration of the molecule known to bind the target molecule is greater than the second concentration of the molecule known to bind the target molecule, where a ratio of greater than 1 indicates that the test compound binds uncompetitively to the target molecule. As another example, determining if the test compound is an uncompetitive inhibitor of the target molecule can be determined by measuring the ratio of the activity of the target molecule in the presence of a concentration of test compound and a first concentration of an agonist of the target molecule to the activity of the target molecule in the presence of the same concentration of test compound and a second concentration of an agonist of the target molecule, where the first concentration of agonist is greater than the second concentration of agonist, where a ratio of greater than 1 indicates that the test compound is an uncompetitive inhibitor of the target molecule.

As another example, determining if the test compound binds uncompetitively to the target molecule can be accomplished by comparing the binding of the test compound in the presence of the same concentration of test compound and different expression levels of the target molecule. As another example, determining if the test compound is an uncompetitive inhibitor of the target molecule can be accomplished by comparing the activity of the target molecule in the presence of the same concentration of test compound and different expression levels of the target molecule.

As another example, determining if the test compound binds uncompetitively to the target molecule can be determined by measuring the ratio of binding to the target molecule in the presence of a concentration of test compound and a first expression level of the target molecule compared to binding to the target molecule in the presence of the same concentration of test compound and a second expression level of the target molecule, where the first expression level of the target molecule is greater than the second expression level of the target molecule, where a ratio of greater than 1 indicates that the test compound binds uncompetitively to the target molecule. As another example, determining if the test compound is an uncompetitive inhibitor of the target molecule can be determined by measuring the ratio of the activity of the target molecule in the presence of a concentration of test compound and a first expression level of the target molecule to the activity of the target molecule in the presence of the same concentration of test compound and a second expression level of the target molecule, where the first expression level of the target molecule is greater than the second expression level of the target molecule, where a ratio of greater than 1 indicates that the test compound is an uncompetitive inhibitor of the target molecule.

The off-rate can be considered a fast off-rate if the off-rate has a ratio of 2:1, 3:1, 4:1, 5:1, 6:1, 7:1, 8:1, 9:1, 10:1, 15:1, 20:1, 25:1, 30:1, 35:1, 40:1, 50:1, 60:1, 70:1, 80:1, 90:1, 100:1, 150:1, 200:1, 300:1, 400:1, 500:1, 750:1, 1000:1, 2000:1, 3000:1, 4000:1, 5000:1, 7500:1, 10⁴: 1, 10⁵: 1, 10⁶: 1, or 10⁷: 1 to the on-rate. The off-rate can be considered a fast off-rate if the off-rate has a ratio of at least 2:1, 3:1, 4:1, 5:1, 6:1, 7:1, 8:1, 9:1, 10:1, 15:1, 20:1, 25:1, 30:1, 35:1, 40:1, 50:1, 60:1, 70:1, 80:1, 90:1, 100:1, 150:1, 200:1, 300:1, 400:1, 500:1, 750:1, 1000:1, 2000:1, 3000:1, 4000:1, 5000:1, 7500:1, 10⁴: 1, 10⁵: 1, 10⁶: 1, or 10⁷: 1 to the on-rate. The off-rate can be considered a fast off-rate if the off-rate has a ratio of 2:1, 3:1, 4:1, 5:1, 6:1, 7:1, 8:1, 9:1, 10:1, 15:1, 20:1, 25:1, 30:1, 35:1, 40:1, 50:1, 60:1, 70:1, 80:1, 90:1, 100:1, 150:1, 200:1, 300:1, 400:1, 500:1, 750:1, 1000:1, 2000:1, 3000:1, 4000:1, 5000:1, 7500:1, 10⁴: 1, 10⁵: 1, 10⁶: 1, or 10⁷: 1 to the apparent affinity. The off-rate can be considered a fast off-rate if the off-rate has a ratio of at least 2:1, 3:1, 4:1, 5:1, 6:1, 7:1, 8:1, 9:1, 10:1, 15:1, 20:1, 25:1, 30:1, 35:1, 40:1, 50:1, 60:1, 70:1, 80:1, 90:1, 100:1, 150:1, 200:1, 300:1, 400:1, 500:1, 750:1, 1000:1, 2000:1, 3000:1, 4000:1, 5000:1, 7500:1, 10⁴: 1, 10⁵: 1, 10⁶: 1, or 10⁷: 1 to the apparent affinity.

As another example, the test compound can be a modified form of a prior test compound. The prior test compound can be, for example, determined to bind uncompetitively to the target molecule, determined to be an uncompetitive inhibitor of the target molecule, or both. The prior test compound can be a modified form of a second prior test compound. The second prior test compound can be, for example, determined to bind uncompetitively to the target molecule, determined to be an uncompetitive inhibitor of the target molecule, or both. The prior test compound can be produced via multiple iterations of modifying an earlier test compound that was determined to bind uncompetitively to the target molecule, determined to be an uncompetitive inhibitor of the target molecule, or both, and determining if the modified test compound binds uncompetitively to the target molecule, is an uncompetitive inhibitor of the target molecule, or both. Different iterations and/or different instances of determining if any of the test compounds binds uncompetitively to the target molecules and/or different iterations and/or different instances of determining if any of the test compounds is an uncompetitive inhibitor of the target molecule are accomplished with the same type of assays, different types of assays, or a combination.

Test compounds can be produced and/or identified in a variety of ways. For example, the disclosed methods can be performed iteratively with, for example, alterations to the test compound used in some or each of the iterations and/or alterations to some or all of the assays or techniques in each of some of the iterations. Such forms of the disclosed methods can be preceded by, interspersed with, and/or followed by steps, manipulations, uses, etc. that are not iterations of the assays and techniques in the iterative steps or phases. For example, following the identification of a test molecule as a pathologically-activated compound in an iterative process (or in a non-iterative process), the pathologically-activated compound can be produced. As another example, following the identification of a test molecule as a pathologically-activated compound in an iterative process (or in a non-iterative process), the pathologically-activated compound can be administered to a subject.

As another example, the method can comprise determining if a first test compound binds uncompetitively to a target molecule and/or determining if a first test compound uncompetitively inhibits the target molecule, where if the first test compound binds uncompetitively to the target molecule and/or the first test compound uncompetitively inhibits the target molecule, then the first test compound is modified to produce a second test compound. The determinations can then be repeated using the second test compound. This iterative process can be continued multiple times. In the last iteration, if the final test compound binds uncompetitively to the target molecule and/or the final test compound uncompetitively inhibits the target molecule, then the final test compound is identified as a pathologically-activated targeting compound.

The test molecule can have an apparent affinity for the target molecule of about 100 nM or greater, an apparent affinity for the target molecule of about 1 μM or greater, an apparent affinity for the target molecule of about 10 μM or greater, or an apparent affinity for the target molecule of about 100 μM or greater. The target molecule can be, for example, a receptor, a neuroreceptor, a G-coupled receptor, a channel receptor, or an or AMPA (α-amino-3-hydroxyl-5-methyl-4-isoxazole-propionate)-, kainate-, or N-methyl-D-aspartate (NMDA)-sensitive glutamate receptor.

As another example, determining if a test compound binds uncompetitively to a target molecule can be performed simultaneously on 96 or more test compounds. As another example, determining if a test compound is an uncompetitive inhibitor of a target molecule can be performed simultaneously on 96 or more test compounds. As another example, determining if a test compound binds uncompetitively to a target molecule can be accomplished using a high throughput screen (HTS). As another example, determining if a test compound is an uncompetitive inhibitor of a target molecule can be accomplished using a high throughput screen.

Methods to determine competitive, noncompetitive and uncompetitive binding are well known in the art. Nonlimiting examples of techniques that can be used to determine competitive, noncompetititve and uncompetitive binding are described below. For example, the kinetics of noncompetitive and uncompetitive components of antagonism can be analyzed using a rapid superfusion system. The mechanism and kinetics of memantine of NMDA-activated channels was determined using a rapid superfusion system and is discussed by Chen et al. (J. Physiol. 499, 27-46 (1997)) which is hereby incorporated by reference. Furthermore, the dissociation (off-rate) rate of an open-channel blocker can be determined using the techniques described by Chen et al. (J. Physiol. 499, 27-46 (1997)). Other examples of determining the kinetics of noncompetitive and uncompetitive components are discussed by Lipton, S. A. et al. (N. Engl. J. Med. 330, 613-622 (1994)), Lipton, S. A. (Trends Neurosci 16, 527-532 (1993)), Chen, H.-S. V. et al. (J. Neurosci. 12, 4427-4436 (1992)), Chen, H.-S. V. et al. (Neuroscience 86, 1121-1132 (1998)) which are hereby incorporated by reference.

As an example of kinetics analysis, the binding kinetics of memantine, an uncompetitive antagonist, are described.

$$Channel + MEM ↔ {{Channel}^{{k}_{on}}}_{{k}_{off}} MEM ( Blocked channel ) \qquad ( 1 )$$

This simple bimolecular scheme predicts that the macroscopic blocking and unblocking actions of memantine (MEM) proceeds with exponential relaxation. The macroscopic pseudo-first order rate constant of blocking (kₒₙ) depends linearly on memantine concentration (as well as a constant, A), and the macroscopic unblocking rate (k_{off}) is independent of memantine concentration ([MEM]).

kₒₙ=A·[MEM] (2)

k_{off}: [MEM] independent (3)

These predictions were borne out experimentally (Chen, H.-S. V. and Lipton, S. A. J. Physiol. (Lond.) 499, 27-46 (1997)). Both the macroscopic blocking and unblocking processes could be well fitted by a single exponential function. The macroscopic on-rate constant is related to the reciprocal of the measured time constant, which for onset (τₒₙ) is the sum of the pseudo-first order blocking rate constant (kₒₙ) and unblocking constant (k_{off}). The unblocking rate constant (k_{off}) is the reciprocal of the measured macroscopic unblocking time constant (k_{off}). These transformations lead to Equations (4) and (5):

kₒₙ=1/τₒₙ−1/τ_{off} (4)

k_{off}=1/τ_{off} (5)

The kon calculated from Equation 4 increased linearly with memantine concentration with a slope factor of 0.4±0.03 106 M−1 s−1 (mean±s.d.), while the k_{off} from Equation 5 remained relatively constant with a Y-axis intercept of 0.44±0.1 s−1 (Chen, H.-S. V. and Lipton, S. A. J. Physiol. (Lond.) 499, 27-46 (1997)). Here a rapid method was used to validate this result by estimating the apparent dissociation constant (Ki) at equilibrium for memantine action from the following equation:

Ki=k_{off}/(kₒₙ/[MEM]) (6)

Here it was found empirically that memantine was a relatively low-affinity (apparent affinity of ˜1 μM) open-channel blocker of the NMDAR-coupled ion channel, and a major component of the affinity was determined by k_{off} at clinically relevant concentrations in the low micromolar range.

### B. Targeted S-Nitrosylation or Prevention of S-Nitrosylation

Post-translational modification of proteins, such as phosphorylation, sulfation (sulfination), and sumoylation, can be harnessed for therapeutic purposes where such modifications decrease excessive, pathological activity of the protein. However, post-translational modifications are persistent owing to the fact that they involve covalent reactions rather than pharmacological interactions. It is therefore useful to induce such modifications only in appropriate situations, such as in damaged regions of the brain of individuals suffering from the conditions listed above, for whom neuroprotection would be beneficial.

An important type of post-translational modification is S-nitrosylation, in which a nitric oxide (NO) group reacts with a critical cysteine thiol to regulate protein activity (Hess, D. et al. Nature Rev. Mol. Cell Biol. 6, 150-166 (2005); Stamler, J. Science 258, 1898-1902 (1992)). For example, S-Nitrosylation of the NMDAR is able to downregulate excessive activity of this receptor and can thereby provide neuroprotection in animal models of hypoxia or ischaemia and of neurodegenerative conditions (Choi, Y. B. et al. Nature Neurosci. 3, 15-21 (2000); Lei, S. Z. et al. Neuron 8, 1087-1099 (1992); Lipton, S. A. et al. Nature 364, 626-632 (1993); Kim, W. K. et al. Neuron 24, 461-469 (1999); Takahashi, H. et al. Neuron 53, 53-64 (2007)).

Importantly, hypoxia facilitates the S-nitrosylation of NMDARs (Choi, Y. B. et al. Nature Neurosci. 3, 15-21 (2000); Lei, S. Z. et al. Neuron 8, 1087-1099 (1992); Lipton, S. A. et al. Nature 364, 626-632 (1993); Kim, W. K. et al. Neuron 24, 461-469 (1999); Takahashi, H. et al. Neuron 53, 53-64 (2007)). In other words, in hypoxic conditions lower amounts of NO are required to effect S-nitrosylation, and inhibition of NMDAR activity by S-nitrosylation is more effective. It is therefore possible for NO donors to preferentially react with the NMDAR in these conditions.

One species of nitric oxide (NO) donor that can be used to decrease NMDAR activity is the alkyl nitrate nitroglycerin. Unlike many other NO donors, alkyl nitrates do not have potential NO-associated neurotoxic effects as they do not directly generate the free radical NO^{·} (the ‘dot’ representing a free electron in the out pi molecular orbital) that could otherwise contribute to neurodegeneration (Lipton, S. A. Nature Rev. Drug Discov. 5, 160-170 (2006); Beckman, J et al. Proc. Natl. Acad. Sci. USA 87, 1620-1624 (1990); Dawson, V. et al. Proc. Natl. Acad. Sci. USA 88, 6368-6371 (1991); Lipton, S. A. et al. Nature 364, 626-632 (1993)). Instead, alkyl nitrates donate NO in the form of a nitro group, —NO₂⁻, which is a relatively safe donor of ‘NO’ in terms of avoiding neurotoxicity. It is also useful for the nitro group to be targeted to the appropriate receptor, in this case the NMDAR; otherwise other effects of NO such as dilatation of blood vessels and consequent hypotension could result.

Recently, memantine has been used to target NO to the NMDAR, and drugs known as NitroMemantines have been developed specifically for this purpose (Lipton, S. A. Nature Rev. Drug Discov. 5, 160-170 (2006)). They differ from memantine in that a nitro group is tethered to the memantine moiety. These drugs work like a guided missile: because of the action of the memantine moiety, NitroMemantines preferentially enter NMDAR-associated channels that are excessively open, which is the case when neurons are depolarized because they have been metabolically, chemically or physically compromised. NitroMemantines then deliver their payload, an NO group, to provide additional blockade of hyperfunctioning NMDARs beyond that offered by memantine alone (Lipton, S. A. Nature Rev. Drug Discov. 5, 160-170 (2006)). In preliminary studies, these drugs have been shown to be more effective than memantine alone in curbing excessive NMDAR-associated channel activity (Lipton, S. A. Nature Rev. Drug Discov. 5, 160-170 (2006)).

For targeted S-nitrosylating compounds, for example, when a pathological condition exists the target molecule could be in a particular state to which the pathologically-activated compound differentially binds or is differentially targeted, whereupon the pathologically-activated compound has an effect on the target molecule. Thus, in this example, the target molecule is itself in a pathological state. The S-nitrosylating compound can be, for example, an uncompetitive binder of the target molecule, thus providing S-nitrosylation when the target molecule is abnormally active or abnormally expressed. The effect of the pathologically-activated compound could be, for example, via the pathologically-activated compound chemically reacting with the target molecule, another molecule associated with or near the target molecule, or a cell or tissue associated with or near the target molecule. For example, the pathologically-activated compound can be linked to a nitro group that nitrosylates the target molecule, thus affecting the activity of the target molecule.

S-nitrosylation is a function of the ability of the pathologically-activated compound to S-nitrosylate the target molecule and/or other molecules in the vicinity of the target molecule. Targeting of the pathologically-activated compound can make use of targeting compounds that preferentially or differentially bind target molecules under pathological conditions. Thus, for example, compounds that bind target molecules uncompetitively can be used for S-nitrosylating pathologically-activated compounds. All of the methods and features discussed elsewhere herein for uncompetitive binding compounds can be used with the S-nitrosylating strategy. For example, pathologically-activated compounds can be formed by linking a nitro group to a pathologically-activated targeting compound. Thus, for this strategy, a nitro group can be linked to the pathologically-activated targeting compound.

Nitric oxide (NO) is a signaling molecule implicated in regulation of many biological processes, including neurotransmitter release, plasticity, and apoptosis (Dawson, T. M.; Snyder, S. H. Journal of Neuroscience 1994, 14, 5147-5159; Lipton, S. A.; Choi, Y. B.; Pan, Z. H.; Lei, S. Z.; Chen, H. S.; Sucher, N. J.; Loscalzo, J.; Singel, D. J.; Stamler, J. S, Nature 1993, 364, 626-632; Melino, G.; Bernassola, F.; Knight, R. A.; Corasaniti, M. T.; Nistico, G.; Finazzi-Agro, A. Nature 1997, 388, 432-433). The chemical reactions of NO are largely dictated by its redox state (Stamler, J. S. Cell 1994, 78, 931-936). NO has been shown to modulate the biological activity of many proteins by reacting with cysteine thiol to form an S-nitrosylated derivative. Such reactions regulate the activity of circulating, membrane-bound, cytosolic, and nuclear proteins, including hemoglobin, NMDA receptors, caspases, and NF—B (Jia, L.; Bonaventura, C. et al., Nature 1996, 380, 221-226; Choi, Y. B., et al., Nature Neuroscience 2000, 3, 15-21; Jaffrey, S. R.; Erdjument-Bromage, H.; et al., Nature Cell Biology 2001, 3, 193-197; Matthews, J. R. et al., Nucleic Acids Research 1996, 24, 2236-2242). S-nitrosylation reactions can regulate specific physiologic and pathophysiologic signaling cascades by directly modifying transcription and/or protein function. S-nitrosylation of different protein species can alter their function. For example, cyclooxygenase, thioredoxin, CFTR and p21^{ras} activities are increased, whereas NF—B, caspase and methionine adenosyl transferase activities are inhibited.

An S-nitrosylating agent or compound can function in vivo to react with protein thiol groups, transferring a NO group to the thiol to form an S-nitrosothiol. Suitable nitrosylating agents are described in Feelisch and Stamler, “Donors of Nitrogen Oxides”, Methods in Nitric Oxide Research edited by Feelisch and Stamler, (John Wiley & Sons) (1996), which is hereby incorporated into this application by reference in its entirety, and specifically for its description of nitrosylating agents and their use. S-nitrosylating agents include acidic nitrite, nitrosyl chloride, ethyl nitrite, glutathione, S-nitrosoglutathione, S-nitrosocysteinyl glycine, S-nitrosocysteine, N-acetyl cysteine, S-nitroso-N-acetyl cysteine, nitroglycerine, nitroprusside, nitric oxide, S-nitrosohemoglobin, S-nitrosoglutathione, S-nitroso-N-acetylpenicillamine, S-nitrosocysteine, S-nitroso-gamma-methyl-L-homocysteine, S-nitroso-L-homocysteine, S-nitroso-gamma-thio-L-leucine and S-nitroso-δ-thio-L-leucine and S-nitrosoalbumin. Alkyl nitrates are particularly useful.

Endogenous S-nitrosothiols (SNOs) are naturally occurring proteins in which a sulfur atom from cysteine or homocystine reacts with NO to form an S—NO bond. S-Nitrosocysteine (SNOC) and S-nitrosoglutathione (GSNO) are endogenous S-nitrosylating agents that regulate several cell-signaling cascades via post-translational modification of cellular proteins. This process involves the transfer of an NO⁺ group to a cysteine thiol-residue forming an S-nitrosothiol. Therefore, cellular S-nitrosylation signals are distinct from classical NO-sensitive cGMP-dependent regulation.

S-Nitrosylation of some targets may lead to aberrant function. Such is the case, for example, for protein disulfide isomerase (PDI) (Uehara et al. S-Nitrosylation of protein-disulphide isomerase links protein misfolding to neurodegeneration. Nature 441, 513-517 (2006)) which causes proteins to misfold in neurodegenerative diseases, and for dynamin-related protein 1 (Drp1) whose nitrosylation results in mitochondrial fragmentation, synaptic damage and eventual neuronal loss in neurodegenerative diseases (Cho et al., S-Nitrosylation of Drp1 mediates β-amyloid-related mitochondrial fission and neuronal injury. Science 324, 102-105 (2009)). Disclosed herein is a method for screening using HTS techniques to find lead compounds or ‘hits’ that prevent such aberrant nitrosylation reactions. For this purpose, the target molecule, such as PDI or Drp1, can be exposed to an NO donor such as SNOC and also to a series of test compounds to determine if these test compounds can prevent the S-nitrosylation reaction for occurring. S-Nitrosylation can be monitored by a chemical reaction called the DAN assay, as described herein (Gu, Z. et al. Science 297, 1186-1190 (2002); (Cho et al., S-Nitrosylation of Drp1 mediates β-amyloid-related mitochondrial fission and neuronal injury. Science 324, 102-105 (2009)). We have found that in the DAN assay CuCl₂ can be substituted for the elemental mercury (HgCl₂), which is known to those skilled in the art; this is desirable since copper is less toxic and therefore more amenable to HTS (with less danger to the workers in the laboratory where the robotic screening procedures are performed).

Accordingly, also disclosed are methods for identifying compounds that inhibit S-nitrosylation. For example, the method can be comprised of comparing S-nitrosylation of a target of interest in the presence and absence of a test compound, wherein an amount of S-nitrosylation of the target measured in the presence of the test compound that is less than the amount of S-nitrosylation of the target measured in the absence of the test compound identifies the test compound as an inhibitor of S-nitrosylation of that target. The amount of S-nitrosylation of the target can be measured by detecting conversion of 2,3-diaminonaphthalene (DAN) to 2,3-naphthyltriazole (NAT). Conversion of 2,3-diaminonaphthalene (DAN) to 2,3-naphthyltriazole (NAT) can be accomplished by measuring the fluorescence of NAT. This conversion is mediated by S-nitrosothiol formation due to donation of NO (nitric oxide species). In the context of these methods, the nitric oxide can be released from the S-nitrosylated targets. The nitric oxide can be released, for example, in the presence of by CuCl₂, suitable for high-throughput screening, although the assay was originally conceived of with HgCl₂ (but which is less suitable for high-throughput screening because mercury (Hg) can be quite toxic to the scientists performing the screening procedure). The target of interest can be any protein or molecule that can be S-nitrosylated (which would affect the protein's activity). Of most interest are proteins and other molecules that are aberrantly S-nitrosylated and/or are S-nitrosylated under pathological conditions. Useful targets of interest can be, for example, protein disulfide isomerase (PDI), dynamin-related protein 1 (Drp1), parkin, DJ-1, a MEF2 transcription factor, a matrix metalloproteinase (MMP), GAPDH, COX-2, or a peroxiredoxin (Prx) such as PrxII (McKercher et al., in: Encyclopedia of Life Sciences: Cell Death. Melino G. Vaux D, cads, John Wiley & Sons, 2010, pp. 262-272).

### C. Upregulation of Drug Targets

Certain pathological conditions, such as hypoxia or ischaemia, which can occur in stroke or vascular dementia (Lipton, S. A. et al. N. Engl. J. Med. 330, 613-622 (1994); Choi, D. Neuron 1, 623-634; Meldrum, B et al. Trends Pharmacol. Sci. 11, 379-387 (1990); Rothman, S. et al. Trends Neurosci. 18, 57-58 (1995)), can upregulate drug targets and thereby increase their specificity for a drug. For example, the lack of oxygen in hypoxia or ischaemia activates hypoxia inducible factor 1α (HIF1α) (Jaakkola, P. et al. Science 292, 468-472 (2001); Bruick, R. Science 294, 1337-1340 (2001); Lando, D., et al. Science 295, 858-861 (2002)), which can trigger the transcription of various target genes, including those for erythropoietin (EPO) and its receptor in the brain (Digicaylioglu, M. et al. Nature 412, 641-647 (2001); Maiese, K. et al. JAMA 293, 90-95 (2005); Brines, M. Nature Rev. Neurosci. 6, 484-494 (2005); Lipton, S. Et al. N. Engl. J. Med. 350, 2516-2517 (2004); Bernaudin, M. et al. J. Cereb. Blood Flow Metab. 19, 643-651 (1999)). EPO, which was originally discovered as a hormone that is produced by the kidney in response to hypoxia (Jelkmann, W. Physiol. Rev. 72, 449-489 (1992)), promotes the formation of red blood cells (erythrocytes) (Finch, C. A. Blood 60, 1241-1246 (1982)), thus increasing the oxygen-carrying capacity of the blood.

It was shown that EPO is also produced in the brain in response to hypoxia (Digicaylioglu, M. et al. Proc. Natl. Acad. Sci. USA 92, 3717-3720 (1995); Masuda, S. et al. J. Biol. Chem. 268, 11208-11216 (1993); Bernaudin, M. et al. Glia 30, 271-278 (2000); Morishita, E. et al. Neuroscience 76, 105-116 (1997)). Moreover, in an analogous fashion to its effect on red blood cells, EPO induces proliferation of neural progenitor cells. It also protects neurons from damage resulting from excessive NMDA or glutamate levels and from insults involving free-radical-mediated damage (Digicaylioglu, M. et al. Nature 412, 641-647 (2001); Maiese, K. et al. JAMA 293, 90-95 (2005); Brines, M. Nature Rev. Neurosci. 6, 484-494 (2005); Lipton, S. A. et al. N. Engl. J. Med. 350, 2516-2517 (2004)). The amount of endogenous EPO that is produced in the brain in response to hypoxia does not elicit maximal neuroprotection, as in both mice and humans administration of additional EPO can provide further neuroprotection (Bernaudin, M. et al. J. Cereb. Blood Flow Metab. 19, 643-651 (1999)).

Importantly, in mice hypoxia or ischaemia has been shown to also increase the number of EPO receptors (Bernaudin, M. et al. J. Cereb. Blood Flow Metab. 19, 643-651 (1999), which are located on neurons (Digicaylioglu, M. et al. Nature 412, 641-647 (2001). Hence, exogenous EPO can be pathologically targeted because its receptor is upregulated during hypoxia and in other forms of injury. In other words, exogenous EPO is an example of a PAT drug whose target is upregulated when needed—during hypoxia.

Expression of target molecules for pathologically-activated compounds could be induced, upregulated or overexpressed under pathological conditions (such as in disease states) such that the pathologically-activated compound differentially accumulates where the induced, upregulated or overexpressed target molecule is induced, upregulated or overexpressed, whereupon the pathologically-activated compound has a differential effect on the target molecule due to the differential accumulation. Thus, in this example, the target molecule is not (necessarily) in a pathological state. Rather, the normal (or pathological) form of the target compound is upregulated or overexpressed. For upregulated and overexpressed target molecules, pathologically-activated compounds can be targeted, for example, using a normal ligand or binding partner for the target molecule. Compounds that bind to the target molecule but that have a high off-rate (and thus, relatively low affinity for the target compound) are also useful forms of pathologically-activated compounds for targeting upregulated and overexpressed target molecules. Thus, for example, the pathologically-activated compound can be an uncompetitive binder of the target molecule, thus differentially binding and affecting the upregulated or overexpressed target molecule.

For this strategy, targeting of the pathologically-activated compound can make use of targeting compounds that preferentially or differentially bind target molecules under pathological conditions. Thus, for example, compounds that bind target molecules uncompetitively can be used for pathologically-activated compounds. All of the methods and features discussed elsewhere herein for uncompetitive binding compounds can be used with this strategy. The target molecule can have increased activity relative to a target molecule that is not activated. The target molecule can be a target molecule expressed at a higher level than a normal or comparison level. A normal or comparison level is a level that is, for example, normal for comparable cells and/or tissues that are not in a pathological or diseased state or that are in a subject note in a relevant pathological or disease state, or that is based on an average level in cells, tissues, or subjects not in a relevant pathological or disease state. In some forms, each copy of the target molecule expressed at a higher level than a normal or comparison level individually can have activity substantially the same as a copy of the target molecule expressed at the normal or comparison level, where the collective activity of the target molecule expressed at a higher level than a normal or comparison level is greater than the collective activity of the target molecule expressed at the normal or comparison level. By “collective activity” is meant the activity of all of the copies of the target molecule present in the, for example, cell, cells, tissue, or tissues of interest (as may be relevant).

### D. Target-Induced Drug Activation

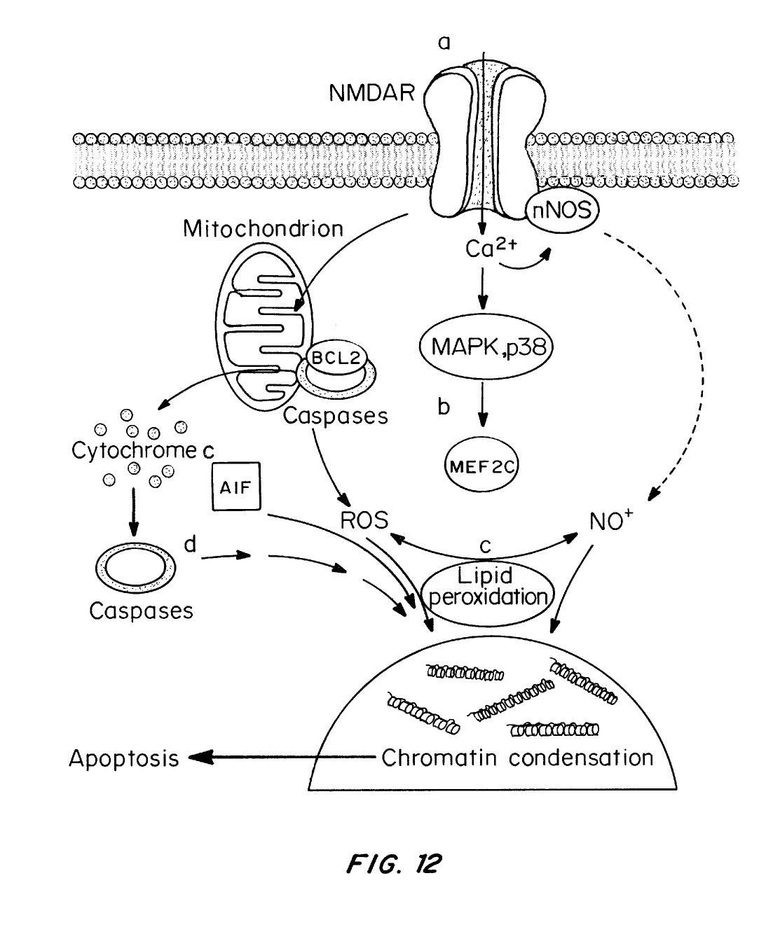

Another PAT mechanism is activation of the drug by its target. Here, an inhibitory drug remains inactive until it interacts with its target. For example, the target could be an enzyme that is pathologically expressed or overactive, and the drug is only activated on binding to the enzyme. Thus, abnormally increased levels or pathological activity of the enzyme results in activation of the drug, which in turn inhibits enzyme action in a feedback-type manner. Such target-induced activation can be considered a form of mechanism-based inhibition (see FIG. 10) (Ikejiri, M. et al. J. Biol. Chem. 280, 33992-34002 (2005); Gu, Z. et al. Science 297, 1186-1190 (2002); Gu, Z. et al. J. Neurosci. 25, 6401-6408 (2005)).

An example of such enzymes are the matrix metalloproteinases (MMPs). Abnormal activation of MMPs, particularly the subtypes MMP2 and MMP9, which are gelatinases located in the extracellular matrix, contribute to damage to the blood-brain barrier and neurons, and are involved in the neuropathology of stroke, Alzheimer's disease, multiple sclerosis, HIV-associated dementia and glaucoma (Gu, Z. et al. Science 297, 1186-1190 (2002); Gu, Z. et al. J. Neurosci. 25, 6401-6408 (2005); Yong, V. Nature Rev. Neurosci. 2, 502-511 (2001); Yong, V. Nature Rev. Neurosci. 6, 931-944 (2005); Lukes, A., et al. Mol. Neurobiol. 19, 267-284 (1999); Yang, Y., et al. J. Cereb. Blood Flow Metab. 27, 697-709 (2007); Jian Liu, et al. Free Radic. Biol. Med. 39, 71-80 (2005); Campbell, I. et al. Trends Neurosci. 22, 285-287 (1999); Montaner, J. et al. Stroke 32, 1759-1766 (2001); Romanic, A. et al. Stroke 29, 1020-1030 (1998); Asahi, M. et al. J. Cereb. Blood Flow Metab. 20, 1681-1689 (2000); Asahi, M. et al. J. Neurosci. 21, 7724-7732 (2001); Gasche, Y. et al. J. Cereb. Blood Flow Metab. 19, 1020-1028 (1999); Heo, J. H. et al. J. Cereb. Blood Flow Metab. 19, 624-633 (1999); Zhao, B. Q. et al. Nature Med. 12, 441-445 (2006)). It was recently shown that these conditions induce NO, which then activates MMPs (Gu, Z. et al. Science 297, 1186-1190 (2002); Gu, Z. et al. J. Neurosci. 25, 6401-6408 (2005)). In turn, excessive MMP activation can contribute to neuronal anoikis, which is a form of apoptotic death caused by detachment of cells from degraded extracellular matrix (Gu, Z. et al. Science 297, 1186-1190 (2002); Gu, Z. et al. J. Neurosci. 25, 6401-6408 (2005)).

In an example of mechanism-based inhibition, the inhibitory drug SB-3CT (3-(4-phenoxyphenylsulphonyl)-propylthiirane) is activated by the increasing availability of the catalytic site of MMP2 or MMP9 (Ikejiri, M. et al. J. Biol. Chem. 280, 33992-34002 (2005); Gu, Z. et al. Science 297, 1186-1190 (2002); Gu, Z. et al. J. Neurosci. 25, 6401-6408 (2005)). Thus, pathologically high activity of the enzyme MMP at the catalytic site results in greater activity of SB-3CT, which inhibits the enzyme, resulting in a form of feedback inhibition that turns off pathological MMP activity. The rapid non-covalent binding of the drug to the catalytic site of MMP is followed by a slower covalent reaction that provides this mechanism-based form of inhibition (Ikejiri, M. et al. J. Biol. Chem. 280, 33992-34002 (2005); Gu, Z. et al. Science 297, 1186-1190 (2002)). Unlike previous MMP inhibitors that were not specific for any subtype of MMP, this new class of drugs is specific for MMP2 and MMP9.

In this mode (target-induced drug activation) of the disclosed methods, the target molecule itself can change or alter the pathologically-activated compound so that it becomes active or effective. Other characteristics can also be useful. For example, expression of the target molecule could be induced, upregulated or overexpressed under pathological conditions (such as in disease states) such that the pathologically-activated compound differentially accumulates where the induced, upregulated or overexpressed target molecule is induced, upregulated or overexpressed, whereupon the pathologically-activated compound has a differential effect on the target molecule due to the differential accumulation. Thus, in this example, the target molecule is not (necessarily) in a pathological state. Rather, the normal (or pathological) form of the target compound is upregulated or overexpressed. Compounds that bind to the target molecule but that have a high off-rate (and thus, relatively low affinity for the target compound) are useful forms of pathologically-activated compounds for this purpose. Target-induced drug activation is particularly suited to cases where the induced, upregulated or overexpressed target molecule can affect molecules that bind (such as enzymes that chemically alter substrate molecules). The pathologically-activated compound can be, for example, a pro-drug that can be activated by the target molecule. In such cases, the fact that the target molecule is differentially expressed under pathological conditions links the effect of the pathologically-activated compound to the pathological expression of the target molecule.

For this strategy, the target molecule can have increased activity relative to a target molecule that is not activated. The target molecule can be a target molecule expressed at a higher level than a normal or comparison level. In some forms, each copy of the target molecule expressed at a higher level than a normal or comparison level individually can have activity substantially the same as a copy of the target molecule expressed at the normal or comparison level, where the collective activity of the target molecule expressed at a higher level than a normal or comparison level is greater than the collective activity of the target molecule expressed at the normal or comparison level.

### E. Activation of Pro-Drugs by Abnormal Redox States

Regulation of neuronal survival by redox reactions has recently been recognized as a major target for the development of neuroprotective drugs (Satoh, T. et al. Proc. Natl. Acad. Sci. USA 103, 768-773 (2006); Satoh, T. et al. Trends Neurosci 30, 37-45 (2007)). One type of chemical redox reaction that can provide neuroprotection involves electrophilic compounds. Electrophiles, which have electron-deficient carbon centers, react with key cysteine thiols to regulate protein function. Interestingly, certain electrophilic compounds, including natural products such as curcumin, the active ingredient in curry powder, have been shown to protect neurons from the effects of glutamate and free-radical insults (Satoh, T. et al. Proc. Natl. Acad. Sci. USA 103, 768-773 (2006); Satoh, T. et al. Trends Neurosci 30, 37-45 (2007)). These findings have resulted in the development of neuroprotective electrophilic drugs, including prostaglandin derivatives and hydroquinones, which work through activation of antioxidant signaling cascades (Satoh, T. et al. Proc. Natl. Acad. Sci. USA 103, 768-773 (2006); Satoh, T. et al. Trends Neurosci 30, 37-45 (2007); Itoh, K. et al. Free Radic. Biol. Med. 36, 1208-1213 (2004); Kraft, A. D. et al. J. Neurosci. 24, 1101-1112 (2004); Shih, A. Y. et al. J. Neurosci. 25, 10321-10335 (2005)).

However, some electrophiles produce severe side effects and may even cause cell damage, principally because they deplete antioxidants such as glutathione. For example, systemically administrated electrophiles such as 4-HBA (bis(4-hydroxybenzylidene)acetone) might react with thiols like glutathione before reaching their intended targets in the brain (Satoh, T. et al. Proc. Natl. Acad. Sci. USA 103, 768-773 (2006); Satoh, T. et al. Trends Neurosci 30, 37-45 (2007)). One way to avoid this problem is to develop compounds that act as a pro-drug and converts to an electrophile only when it is confronted with oxidizing or damaging free radicals on reaching the intended target. This represents another example of a pathological target that activates its own inhibitor. For example, terpenoids, which possess catechol rings, are pro-electrophilic compounds that can be converted to quinone-type electrophiles by oxidation (see FIG. 11).

Electrophilic compounds can, via redox reactions, induce transcription of many phase 2 enzymes such as heme oxygenase 1 (HO1; also known as HMOX1), which are involved in drug detoxification, redox regulation and neuroprotection (Satoh, T. et al. Proc. Natl. Acad. Sci. USA 103, 768-773 (2006); Satoh, T. et al. Trends Neurosci 30, 37-45 (2007)). This strategy can, for example, be applied in Parkinson's disease. Although oxidative stress plays a crucial role in the progression of Parkinson's disease, it can also be used to activate pro-electrophilic compounds at the target site to provide neuroprotection where it is needed. Thus, a useful strategy against neurodegenerative disorders is to activate electrophilic drugs via the pathological activity that they are intended to combat.

For activation of pro-drugs by abnormal redox states, the pathologically-activated compound can be active only under particular conditions that occur under pathological conditions, whereupon the pathologically-activated compound affects the target molecule only under pathological conditions. For example, many pathological states are associated with oxidizing conditions. A compound that is differentially reactive or that is altered and activated under oxidizing conditions can be used as a pathologically-activated compound for pathological states that includes oxidizing conditions. For example, compounds are known that become electrophiles under oxidizing conditions. Such compounds can be used in or as pathologically-activated compounds.

Pathologically-activated compounds can be made, for example, by linking a redox-activated group to a targeting compound, where the redox-activated group becomes chemically reactive when exposed to oxidizing conditions or the redox-activated group becomes chemically reactive when exposed to reducing conditions. Subject for this strategy can be, for example, subjects in need of inhibition of a target molecule a composition comprising a pathologically-activated compound active only under conditions that occur in a pathological state. The conditions that occur in a pathological state can be oxidizing conditions. In some forms, the pathologically-activated compound can become electrophilic under the conditions that occur in the pathological state. Subjects in need of inhibition of a target molecule refer to subjects that have too much of the target molecule, has target molecules that are too active, or both, generally where such activity is associated with or is a feature of the relevant pathological or disease condition or state. By “active only under conditions that occur in a pathological state” is meant that the compound or molecule exhibits substantial activity under conditions, or as a result of an activity, that are present in or under the relevant pathological or disease conditions or state.

Pro-electrophilic compounds can be pro-drugs that convert to an electrophile by oxidation upon reaching the intended target. For example, terpenoids that possess catechol (1,2-dihydroxybenzene) rings represent pro-electrophilic compounds that can be converted to quinone-type electrophiles by oxidation (Dinkova-Kostova et al., Proc. Natl. Acad. Sci. USA 102:4584-4589, 2005). Terpenoids are modified terpenes (compounds derived biosynthetically from units of isoprene), wherein methyl groups have been moved or removed, or oxygen atoms added.

Similarly, other phenolic compounds can be converted to quinone-type electophilic compounds via oxidation by free radicals. Examples of such reactions are phenolic oxidative coupling reactions which often produce quinone moieties from phenol-type moieties (Smith, M., “Organic Chemistry 2^{nd} Ed” McGraw-Hill Companies, Inc. New York, N.Y., (2002)).

### F. Administration and Treatment

The disclosed pathologically-activated compounds and pathologically-activated targeting compounds can be used to treat diseases and pathological conditions. For example, a pathologically-activated compound can be administered to a subject in need of inhibition of an activated target molecule. The pathologically-activated compound can be any disclosed pathologically-activated compound, including, for example, those identified using the disclosed methods. The pathologically-activated compound can be, for example, active only under conditions that occur in a pathological state. For example, the conditions that occur in a pathological state can be oxidizing conditions. As another example, the pathologically-activated compound can become electrophilic under the conditions that occur in the pathological state.

The subject can be suffering from or at risk for a disease characterized by an activated target molecule. A disease characterized by an activated target molecules refers to a pathological or disease condition or state where the activated target molecule is present (in activated form) in the pathological or disease condition or state and is not substantially present (in activated form) in comparable cells, tissue, etc. when the pathological or disease condition or state is not present or does not exist, for example, under normal conditions. The activated target molecule can have increased activity relative to a target molecule that is not activated. The activated target molecule can be a target molecule that is in a different state or has different potential relative to a target molecule that is not activated. The activated target molecule can be a target molecule expressed at a higher level than a normal or comparison level. In some forms, each copy of the target molecule expressed at a higher level than a normal or comparison level individually can have activity substantially the same as a copy of the target molecule expressed at the normal or comparison level, where the collective activity of the target molecule expressed at a higher level than a normal or comparison level is greater than the collective activity of the target molecule expressed at the normal or comparison level.

### Materials

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a pathologically-activated compound is disclosed and discussed and a number of modifications that can be made to a number of molecules including the pathologically-activated compound are discussed, each and every combination and permutation of pathologically-activated compounds and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Disclosed are pathologically-activated compounds and compounds and compositions for identifying, producing, and using pathologically-activated targeting compounds. Disclosed are pathologically-activated targeting compounds that bind uncompetitively to the target molecule, that are uncompetitive inhibitors of the target molecule, and/or that have a fast off-rate for the target molecule. The pathologically-activated compounds can have a nitro group, a redox-activated group, or both. The redox-activated group can become chemically reactive when exposed to oxidizing conditions or when exposed to reducing conditions.

As another example, the pathologically-activated compounds can have an apparent affinity for the target molecule of about 100 nM or greater, an apparent affinity for the target molecule of about 1 μM or greater, an apparent affinity for the target molecule of about 10 μM or greater, or an apparent affinity for the target molecule of about 100 μM or greater. The target molecule can be any that is altered or activated under pathological conditions, including by being abnormally expressed and/or abnormally activated. The target molecule can also be a molecule the activity of which is involved in the pathological condition. For example, the target molecule can be a receptor, a neuroreceptor, a G-coupled receptor, a channel receptor, or an N-methyl-D-aspartate (NMDA)-sensitive, AMPA-sensitive, or Kainate-sensitive glutamate receptor.

### A. Pathologically-Activated Compounds

A “pathologically-activated compound” is a compound that only has an effect, or has a disproportionate effect, on a target molecule when a pathological condition exists. As described herein, pathologically-activated compounds can have such effects under pathological conditions based on a variety of features. Examples and features of pathologically-activated compounds are also describes elsewhere herein.

A “targeting compound” is a compound that binds to and/or accumulates at the site of a target molecule. A “pathologically-activated targeting compound” is a targeting compound that only binds to and/or accumulates at the site of a target molecule when a pathological condition exists. For example, when a pathological condition exists the target molecule could be in a particular state to which the pathologically-activated targeting compound differentially binds or is differentially targeted. Thus, in this example, the target molecule is itself in a pathological state. As another example, expression of the target molecule could be induced, upregulated or overexpressed under pathological conditions (such as in disease states) such that the pathologically-activated targeting compound differentially accumulates where the induced, upregulated or overexpressed target molecule is induced, upregulated or overexpressed. Thus, in this example, the target molecule is not (necessarily) in a pathological state. Rather, the normal (or pathological) form of the target compound is upregulated or overexpressed.

A pathologically-activated targeting compound can be a pathologically-activated compound. However, not all pathologically-activated compounds are pathologically-activated targeting compound. Pathologically-activated targeting compounds are targeted to a target molecule when pathological conditions exist but may or may not affect the target compound. Pathologically-activated compounds affect the target molecule when pathological conditions exist but may or may not be targeted to the target molecule.

### B. Target Molecules

A “target molecule” is any compound, molecule, or composition that is, or is desired to be, the target of a pathologically-activated compound. Useful target molecules include, for example, molecules that are abnormally activated or expressed in a disease state or under disease conditions. Useful target molecules also include molecules characteristic of target cells or target tissues where an activating disease state of condition is or may be present. An “activating disease state” or “activating disease condition” is a state or condition that occurs in a disease or pathological state or condition that can activate a pathologically-activated compound.

An “activated target molecule” is a target molecule that has increased activity relative to a target molecule that is not activated or is a target molecule that is in a different state or has different potential relative to a target molecule that is not activated. A target molecule expressed at a higher level than a normal or comparison level can be an activated target molecule. A “disproportionate effect” refers to an effect that is 2, 3, 4, 5, 6, 7, 8, 9, 10, 20, 30, 40, 50, or 100 times greater in magnitude than a comparison effect.

### C. Pharmaceutical Compositions and Carriers

The disclosed compositions can be administered in vivo in a pharmaceutically acceptable carrier. By “pharmaceutically acceptable” is meant a material that is not biologically or otherwise undesirable, i.e., the material can be administered to a subject, along with the homing composition, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art. The materials can be in solution, suspension (for example, incorporated into microparticles, liposomes, or cells).

1. Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers can be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The pharmaceutical composition can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### D. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for identification of pathologically-activated compounds, the kit comprising test compounds, target molecules, or both.

### E. Mixtures

Disclosed are mixtures formed by performing or preparing to perform the disclosed method. For example, disclosed are mixtures comprising test compounds and target molecules, pathologically-activated compounds and target molecules, and pathologically-activated compounds and targeted cells or tissue.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

### F. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated. For example, disclosed and contemplated are systems comprising test compounds and assay apparatus.

### G. Data Structures and Computer Control

Disclosed are data structures used in, generated by, or generated from, the disclosed method. Data structures generally are any form of data, information, and/or objects collected, organized, stored, and/or embodied in a composition or medium. Binding kinetics data stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### Uses

The disclosed methods and compositions are applicable to numerous areas including, but not limited to, identifying, testing, producing, and administering pathologically-activated compounds. Other uses include designing modified pathologically-activated compounds based on identified pathologically-activated compounds. Other uses are disclosed, apparent from the disclosure, and/or will be understood by those in the art.

### EXAMPLES

The following example is put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### A. Example 1

### S-Nitrosylated Protein-Disulfide Isomerase Links Protein Misfolding to Neurodegeneration

Stress proteins located in the cytosol or endoplasmic reticulum (ER) maintain cell homeostasis and afford tolerance to severe insults (Ellgaard, L., et al. Science, 286, 1882-1888, (1999); Kaufman, R. J., Genes Dev. 13, 1211-1233, (1999); Patil, C., et al. Curr. Opin. Cell Biol., 13, 349-355, (2001)). In neurodegenerative diseases, several chaperones ameliorate the accumulation of misfolded proteins triggered by oxidative or nitrosative stress, or of mutated gene products (Rao, R. V., et al. Curr. Opin. Cell Biol., 16, 653-662, (2004); Haynes, C. M., et al. Mol. Cell, 15, 767-776, (2004)). Although severe ER stress can induce apoptosis (Kaufman, R. J., Genes Dev. 13, 1211-1233, (1999); Imai, Y., Cell, 105, 891-902, (2001)), the ER withstands relatively mild insults through the expression of stress proteins or chaperones such as glucose-regulated protein (GRP) and protein-disulphide isomerase (PDI), which assist in the maturation and transport of unfolded secretory proteins. PDI catalyses thiol-disulphide exchange, thus facilitating disulphide bond formation and rearrangement reactions (Edman, J. C., et al. Nature, 317, 267-270, (1985); Vuori, K., et al. J. Biol. Chem., 267, 7211-7214, (1992); Song, J. L., et al. Eur. J. Biochem, 231, 312-316, (1995)). PDI has two domains that function as independent active sites with homology to the small, redox-active protein thioredoxin (Edman, J. C., et al. Nature, 317, 267-270, (1985)). During neurodegenerative disorders and cerebral ischaemia, the accumulation of immature and denatured proteins results in ER dysfunction (Gilbert, H. F., “Protein disulfide isomerase”, Methods Enzymol., 290, 26-50, (1998)), but the upregulation of PDI represents an adaptive response to protect neuronal cells (Hu, B. R., et al. J. Neurosci. 20, 3191-3199, (2000); Ko, H. S., et al. J. Biol. Chem., 277, 35386-35392, (2002); Tanaka, S., et al. J. Biol. Chem., 275, 10388-10393, (2000)). Here it is shown that in brains manifesting sporadic Parkinson's or Alzheimer's disease, that PDI is S-nitrosylated, a reaction transferring a nitric oxide (NO) group to a critical cysteine thiol to affect protein function (Uehara et al., Nature 441, 513-517 (2006); Conn, K. J., et al. Brain Res., 1022, 164-172, (2004); Stamler, J. S., Cell, 78, 931-936, (1994); Haendeler, J., Nature Cell Biol., 4, 743-749, (2002)). NO-induced S-nitrosylation of PDI inhibits its enzymatic activity, leads to the accumulation of polyubiquitinated proteins, and activates the unfolded protein response. S-Nitrosylation also abrogates PDI-mediated attenuation of neuronal cell death triggered by ER stress, misfolded proteins or proteasome inhibition. Thus, PDI prevents neurotoxicity associated with ER stress and protein misfolding, but NO blocks this protective effect in neurodegenerative disorders through the S-nitrosylation of PDI.

1. Mammalian PDI

Mammalian PDI has six cysteine residues in all, with four of them representing two thioredoxin-like domains (one near the amino terminus and the other near the carboxy terminus) that contain the Cys-Gly-His-Cys (SEQ ID NO:8) sequence at the active site.

2. Methods and Results

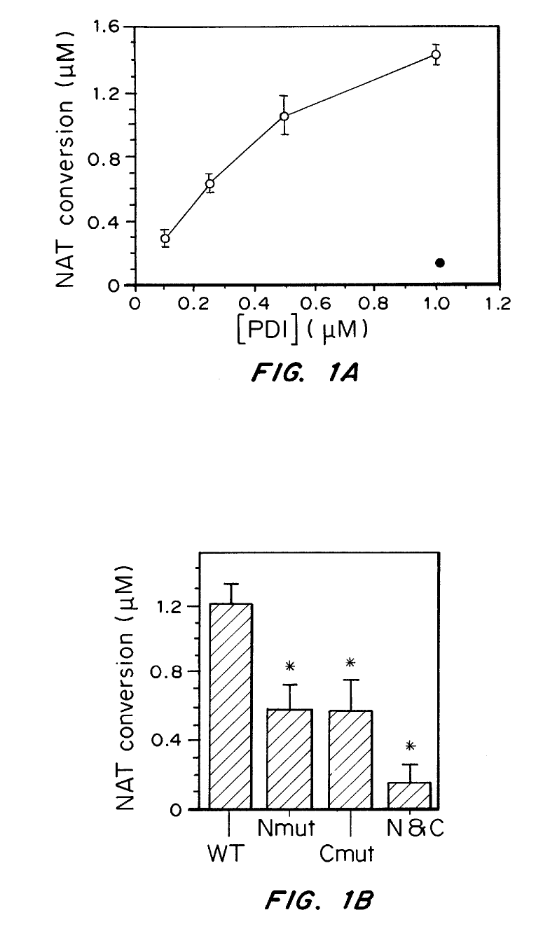

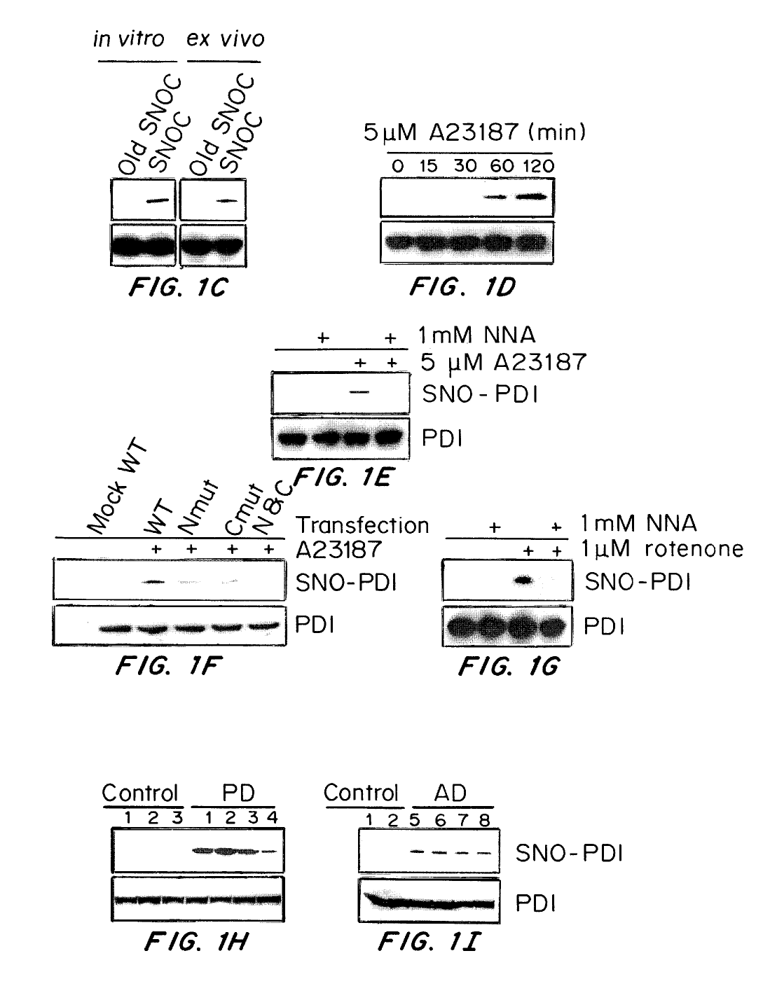

Two independent lines of chemical evidence show that PDI was S-nitrosylated in vitro and in vivo to form an S-nitrosylated protein (SNO-P). First, in a specific fluorescence assay for SNO-P, the reaction of recombinant PDI with the physiological NO donor S-nitrosocysteine (SNOC; FIG. 1a) was demonstrated. This assay detects the formation of SNO-P by the conversion of 2,3-diaminonaphthalene (DAN) to the fluorescent compound 2,3-naphthyltriazole (NAT) (Stamler, J. S., Cell, 78, 931-936, (1994); Sliskovic, I, et al. J. Biol. Chem., 280, 8733-8741, (2005); Gu, Z., Science, 297, 1186-1190, (2002)). SNOC-treated PDI resulted in significant SNO-P formation in a concentration-dependent manner.

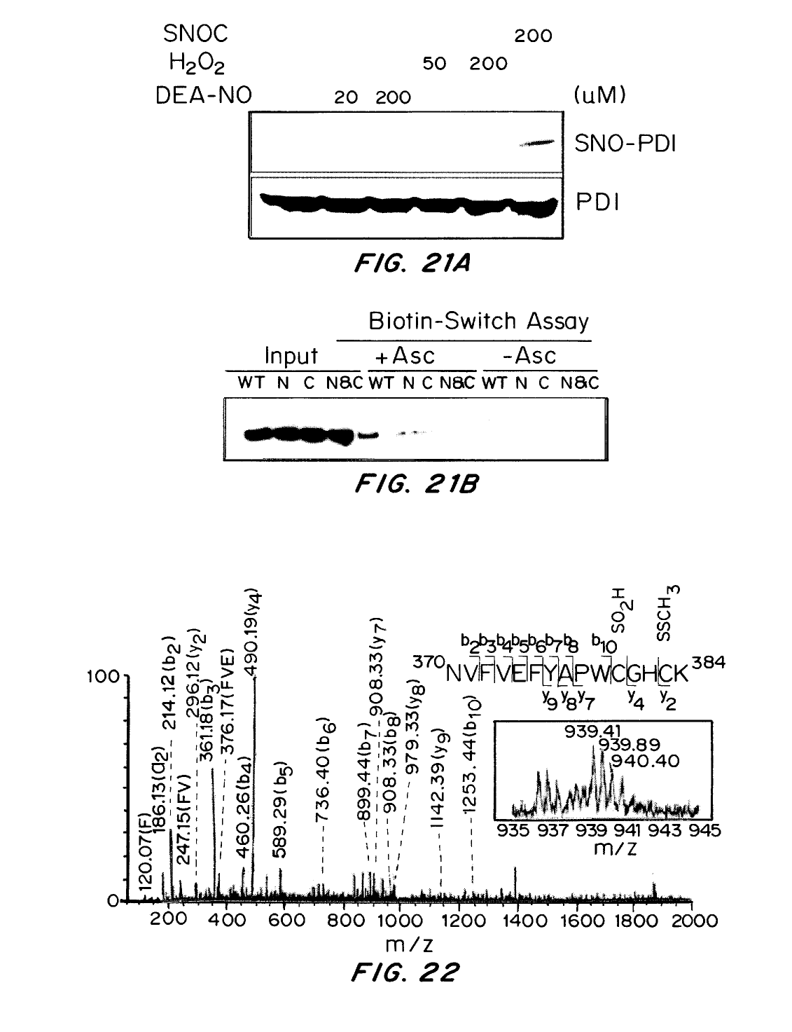

To determine the target site(s) of S-nitrosylation, the DAN assay was performed on immunoprecipitates from HEK-293T cell lysates transfected with wild-type or mutant PDI (cysteine-to-serine mutations in both of the active-site sequences). The fluorescence intensity in this assay of the N-terminal (C36S, C39S) and C-terminal (C383S, C386S) mutants was decreased by about 50% compared with the wild type. The double mutant (N-terminal and C-terminal) was completely devoid of fluorescence, indicating that both thioredoxin-like domains are targets of S-nitrosylation (FIG. 1b). Further, it was shown that PDI in 293T cells was 5-nitrosylated by the biotin-switch method. In this assay, a SNO-P is identified on western blots after replacing SNO with a more stable biotin group by chemical reduction with ascorbate, as described previously (Conn, K. J., et al. Brain Res., 1022, 164-172, (2004); Yao, D., et al. Proc. Natl. Acad. Sci. USA, 101, 10810-10814, (2004); Gu, Z., et al. Science, 297, 1186-1190, (2002)). SNOC markedly enhanced the level of S-nitrosylated PDI (SNO-PDI) in cell lysates or intact cells (FIG. 1c), whereas under the same conditions the NO^{·} donor diethylamine-NO (DEA-NO) or hydrogen peroxide did not (See FIG. 21a). Additionally, in HEK-293 cells stably expressing neuronal NO synthase (nNOS), endogenous PDI was nitrosylated by endogenous NO, and this reaction was inhibited by a NOS inhibitor (FIG. 1d, e). Furthermore, by the biotin-switch assay we identified the cysteine residues that were S-nitrosylated. It was found that endogenous nNOS activity led to the S-nitrosylation of cysteine residues in both thioredoxin-like domains of PDI (See FIG. 1f and FIG. 21b). Moreover, using mass spectrometry it was determined that one of the cysteine residues in the C-terminal thioredoxin-like domain was possibly further oxidized to sulphinic acid (—SO₂H) after exposure to NO (See FIG. 22). These data are consistent with the previous observation that reversible S-nitrosylation can facilitate further oxidation of the same cysteine thiol (Sliskovic, I, et al. J. Biol. Chem., 280, 8733-8741, (2005)).

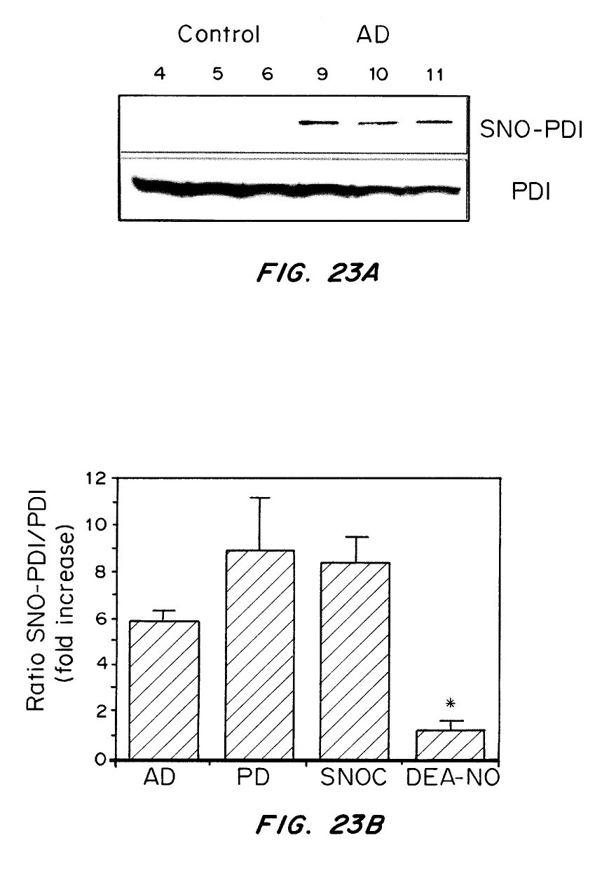

Further, it was investigated if SNO-PDI was produced in neurodegenerative disorders associated with high levels of nitrosative stress and protein misfolding, such as Parkinson's disease (PD). First, dopaminergic SH-SY5Y cells was incubated with the mitochondrial complex I inhibitor rotenone, which is known to induce a parkinsonian phenotype, at least in part, in a NO-dependent fashion (Jaffrey, S. R., Nature Cell Biol., 3, 193-197, (2001)). Exposure to rotenone led to the generation of SNO-PDI in these cells (See FIG. 1g). To extend this finding to humans, PD brains were obtained and examined shortly after death. Evidence showed formation of SNO-PDI in each of four PD brains but not in controls obtained from patients who had died of disorders that were not of central nervous system origin (See FIG. 1h and Table 1). Additionally, brains from another major neurodegenerative disorder associated with protein aggregation and nitrosative stress, Alzheimer's disease (AD), also showed evidence of SNO-PDI (See FIG. 1i, FIG. 23 and Table 1), consistent with the notion that this finding could represent a common denominator linking free-radical stress and protein misfolding.

Table 1 shows a list of human brain subjects used in this study

| | | | | Age at | |
|---|---|---|---|---|---|
| | | | | time of | |
| Subjects | Diagnosis | Brain Region | PMI (hr) | Death | Gender |
| Control 1 | Congestive heart failure | Medial frontal cortex | 8 | 74 | F |
| Control 2 | Complication of abdominal | Temporal cortex | 2 | 71 | M |
| | aortic aneurysm repair | | | | |
| Control 3 | Respiratory failure | Temporal cortex | 6 | 83 | M |
| Control 4 | N/A | Medial frontal cortex | 6 | 79 | F |
| Control 5 | N/A | Medial frontal cortex | 6 | 91 | M |
| Control 6 | Complication of abdominal | Medial frontal cortex | 2 | 71 | M |
| | aortic aneurysm repair | | | | |
| Patient 1 | PD | Temporal cortex | 12 | 88 | M |
| Patent 2 | PD | Temporal cortex | 4 | 77 | F |
| Patient 3 | PD | Temporal cortex | 4 | 77 | F |
| Patient 4 | PD | Temporal cortex | 6 | 75 | F |
| Patient 5 | AD | Medial frontal cortex | 6 | 86 | M |
| Patient 6 | AD | Medial frontal cortex | N/A | 96 | M |
| Patient 7 | AD | Medial frontal cortex | 6 | 88 | M |
| Patient 8 | AD | Medial frontal cortex | 8 | 88 | F |
| Patient 9 | AD | Medial frontal cortex | 6 | 93 | F |
| Patient 10 | AD | Medial frontal cortex | 12 | 88 | F |
| Patient 11 | AD | Medial frontal cortex | 5 | 65 | M |

PMI: Postmortem Interval (From the time of death of freezing of samples)

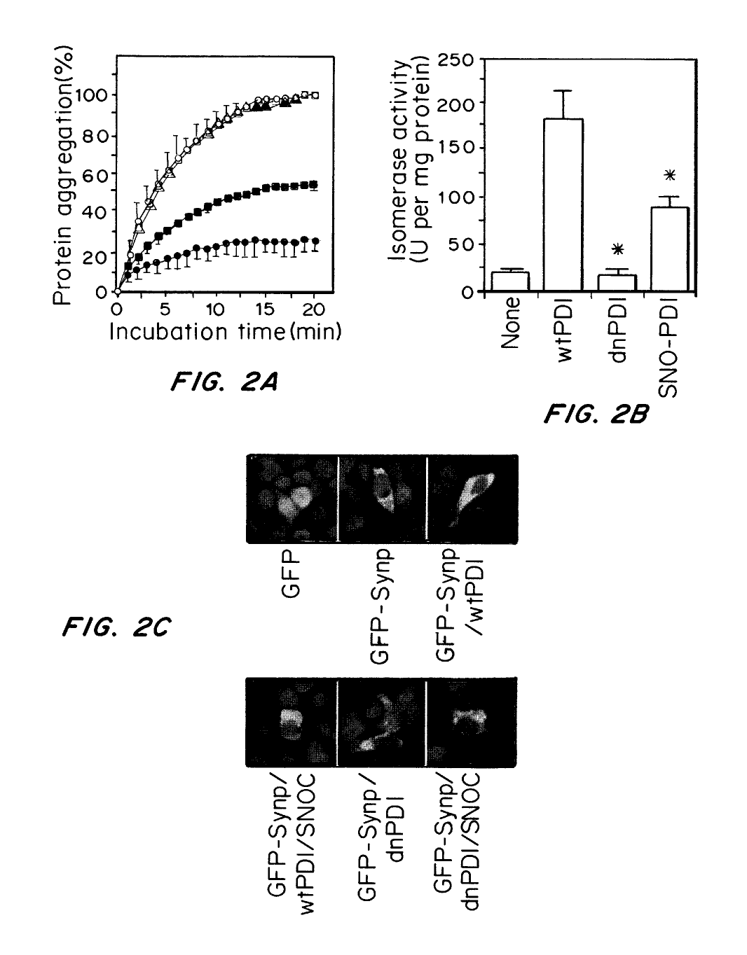

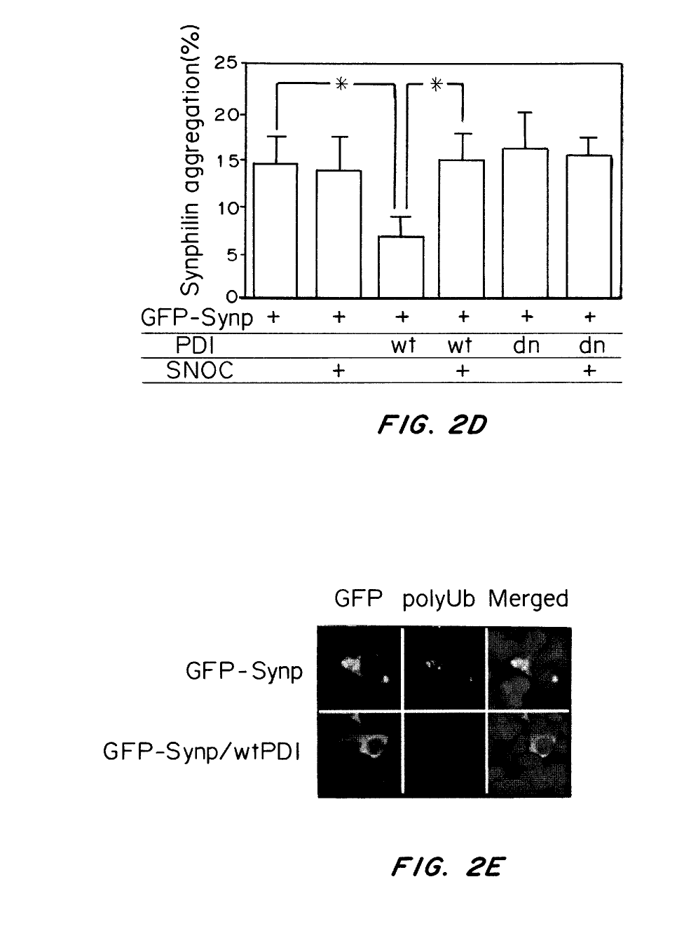

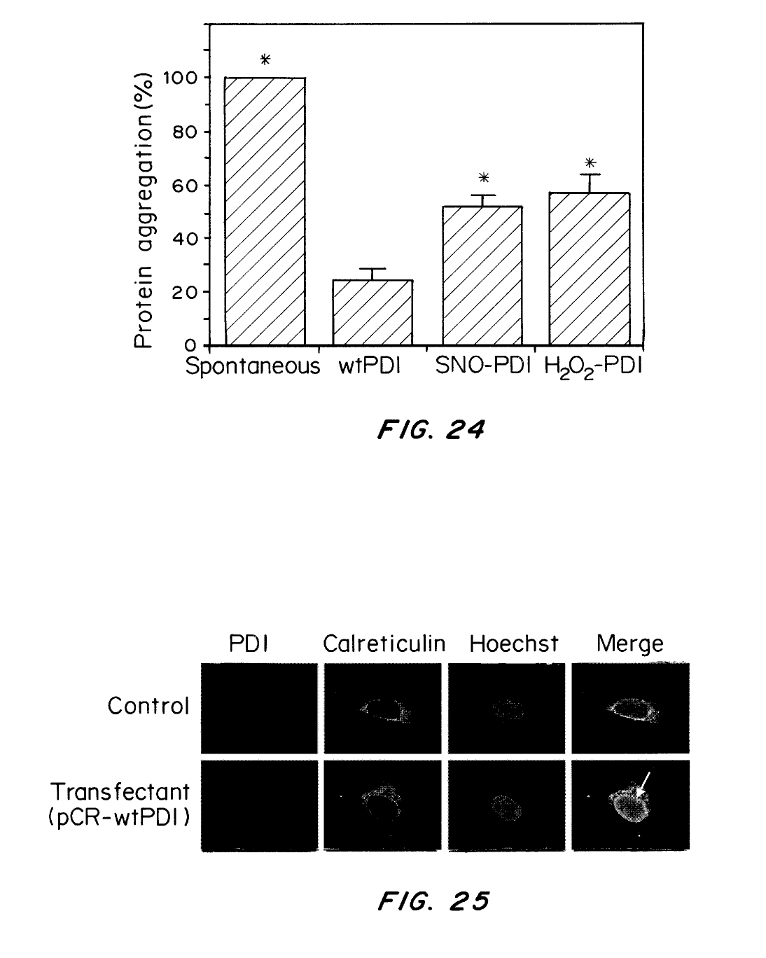

To determine whether S-nitrosylation affects PDI function, PDI chaperone and isomerase activities were monitored. Chaperone activity was assessed in a standard assay by measuring the degree of aggregation of rhodanese induced by guanidinium, as previously described9. Rhodanese aggregation occurred in a time-dependent manner, and incubation with recombinant wild-type PDI, but not dominantnegative PDI (produced by the N-terminal and C-terminal mutant) (Ko, H. S., et al. J. Biol. Chem., 277, 35386-35392, (2002)), suppressed this aggregation by about 80%. S-Nitrosylation of PDI also significantly inhibited this chaperone activity (See FIG. 2a). Further, the isomerase activity was measured in a standard assay that uses as a substrate an inactive form of RNase A containing scrambled disulphide bonds (Song, J. L., et al. Eur. J. Biochem, 231, 312-316, (1995)). PDI catalyses the renaturation (refolding) of this inactive RNase A. Recovery of RNase A activity by wild-type PDI was attenuated about 50% by S-nitrosylation (See FIG. 2b). Thus, S-nitrosylation inhibited the functional activities of PDI. In addition, direct oxidation of PDI by hydrogen peroxide could also decrease its activity (See FIG. 24), indicating that the sulphinated PDI derivative observed by mass spectrometry after exposure to NO (See FIG. 22) can also be pathophysiologically relevant to the inhibition of PDI activity. These results indicate that PDI could function in attenuating protein misfolding and consequent aggregation in neurodegenerative diseases, and that the formation of SNO-PDI might inhibit neuroprotective activity. Moreover, previous work indicates that ER stress can directly or indirectly influence the aggregation of both ER and cytosolic proteins (Rao, R. V., et al. Curr. Opin. Cell Biol., 16, 653-662, (2004); Haynes, C. M., et al. Mol. Cell, 15, 767-776, (2004); Betarbet, R., Nature Neurosci., 3, 1301-1306, (2000)). Therefore, the inhibitory effect of PDI on the aggregation of synphilin-1 was investigated, as observed in Lewy body inclusions in the brains of PD patients. First, it was tested if PDI could prevent the ubiquitinated, Lewy-body-like inclusions that are formed in the cytosol after synphilin-1 overexpression in cultured SH-SY5Y cells (See FIG. 2c). When wild-type PDI was co-expressed with synphilin-1, discrete inclusions were greatly decreased, and instead ubiquitin-negative synphilin-1 was localized diffusely in the cytosol (See FIG. 2c-e). As a control, immunofluorescent staining of transfected SH-SY5Y cells revealed that overexpression of PDI did not alter its predominant intracellular distribution in the ER (See FIG. 25). NO attenuated the protective effect of PDI on synphilin-1 inclusions (FIG. 2d). These findings indicate that PDI is involved in protein folding linked to PD in a NO-sensitive manner.

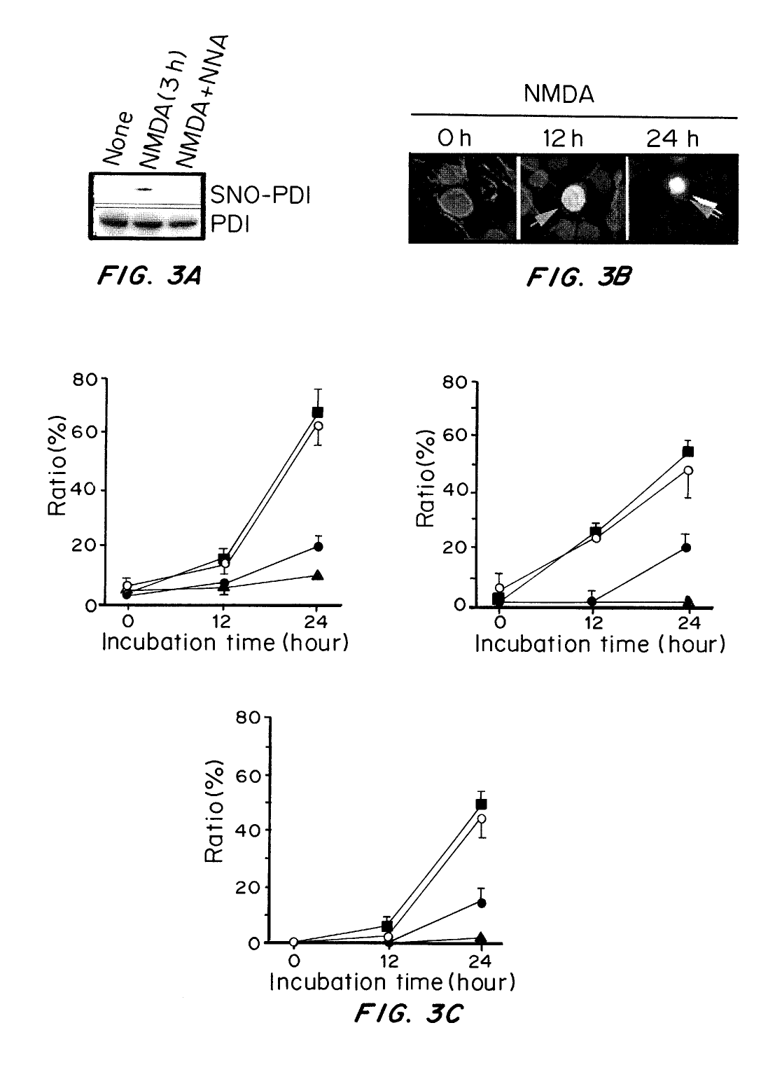

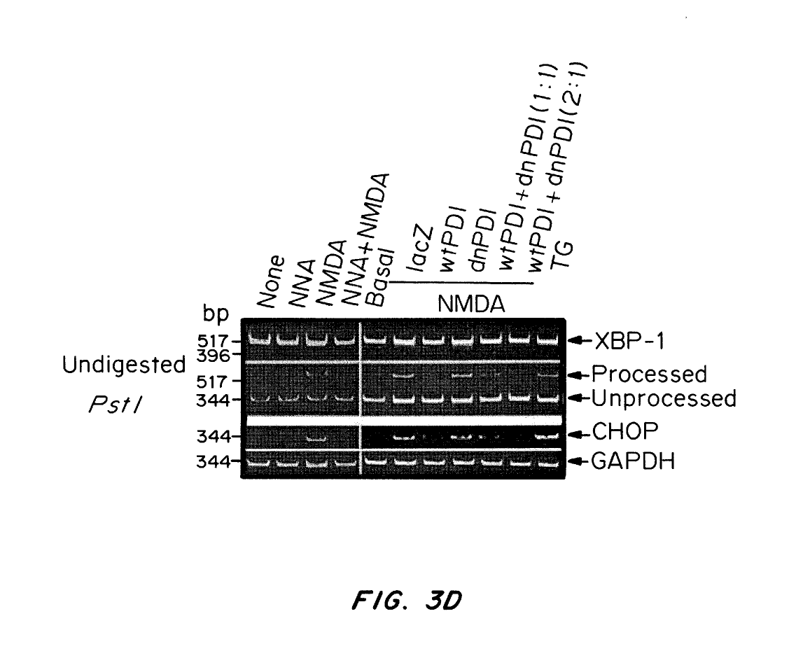

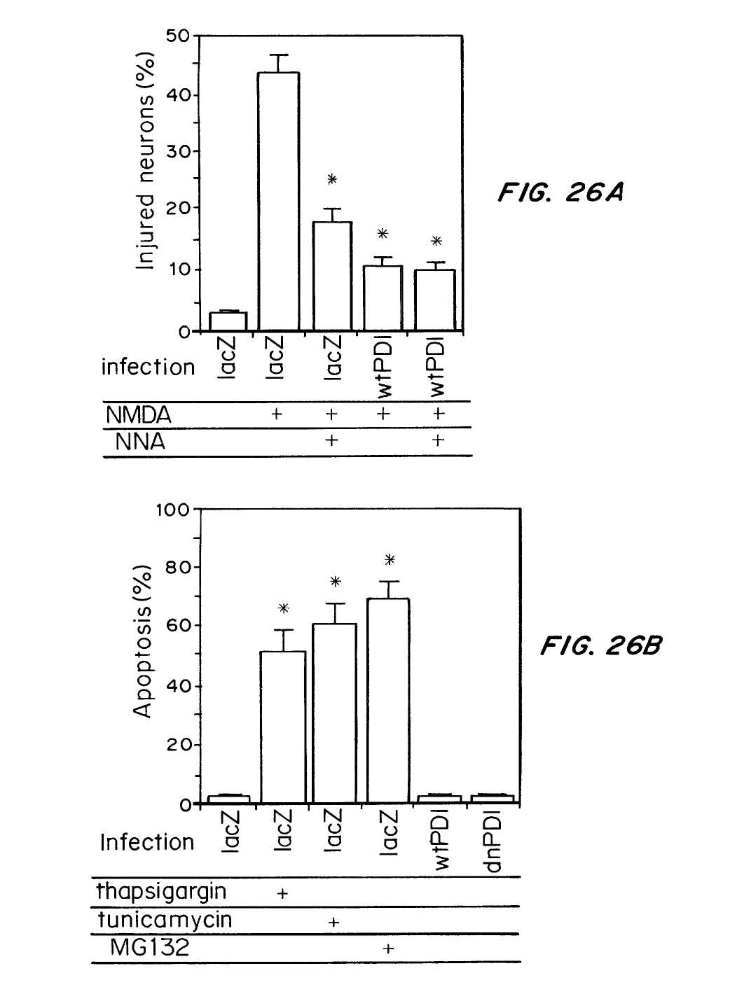

Excitotoxic damage is also thought to have a function in neurodegenerative disorders such as PD by triggering the production of free radicals, including NO, in part through the excessive stimulation of N-methyl-D-aspartate (NMDA)-type glutamate receptors; mild exposure to NMDA results in neuronal apoptosis (Nishikawa, A., Biochem. Biophys. Res. Commun., 318, 435-438, (2004); Bonfoco, E., Proc. Natl. Acad. Sci. USA, 92, 7162-7166, (1995)); Dawson, V. L., et al. Proc. Natl. Acad. Sci. USA, 88, 6368-6371, (1991); Lipton, S. A., et al. N. Engl. J. Med. 330, 613-622, (1994)). Here it was found that exposure of cerebrocortical neurons to NMDA induced SNO-PDI in a NOS-sensitive fashion (See FIG. 3a). Results indicate that abrogation of PDI chaperone and enzymatic activity by S-nitrosylation could contribute to the accumulation of unfolded and consequently polyubiquitinated proteins marked for degradation by the proteasome. Thus, it was examined whether the accumulation of polyubiquitinated proteins occurred in response to NMDA by using a polyubiquitin-specific antibody. Within 12 h of exposure to NMDA, polyubiquitin immunoreactivity was detected in neurons, but the cells remained viable at this time point (See FIG. 3b, c). By 24 h, many of the polyubiquitinated neurons had undergone apoptosis. Overexpression of wild-type PDI decreased the number of apoptotic, polyubiquitinated cells, indicating that PDI has a function in preventing the accumulation of unfolded, polyubiquitinated proteins in response to NMDA insult, and subsequent neuronal cell death. Next the involvement of the unfolded protein response (UPR) signaling pathway that is activated by the accumulation of misfolded proteins or ER dysfunction was investigated. Representing this pathway CHOP mRNA induction and XBP-1 mRNA processing by activated IRE1-a after exposure of cortical cultures to NMDA was detected (Ellgaard, L., Science, 286, 1882-1888, (1999); Kaufman, R. J., Genes Dev. 13, 1211-1233, (1999)). This UPR was attenuated by overexpression of wild-type PDI but not by dominant-negative PDI (See FIG. 3d). Additionally, the NOS inhibitor N-nitro-L-arginine blocked NMDA-induced apoptosis and the UPR, indicating that a pathophysiologically relevant amount of NO was produced under these conditions (See FIG. 3c, d and FIG. 26). Taken together, these findings indicate that NMDA activates a NO-mediated UPR through ER dysfunction, but this dysfunction can be mitigated by PDI activity.

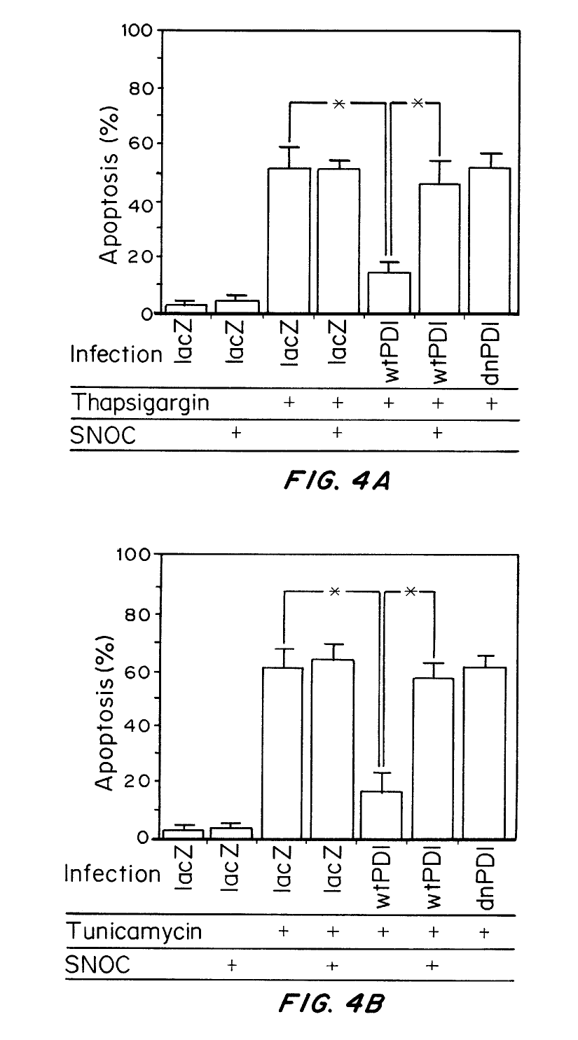

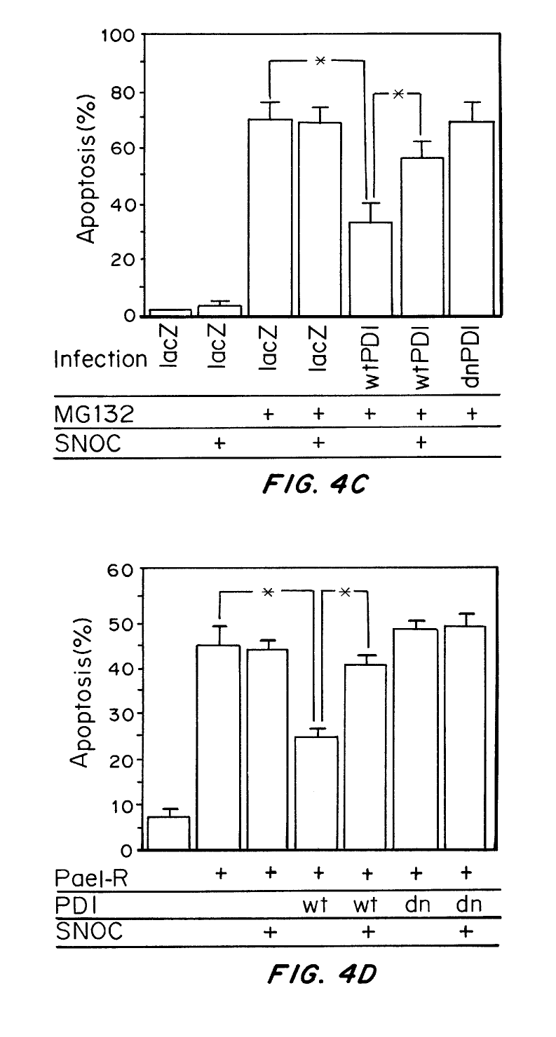

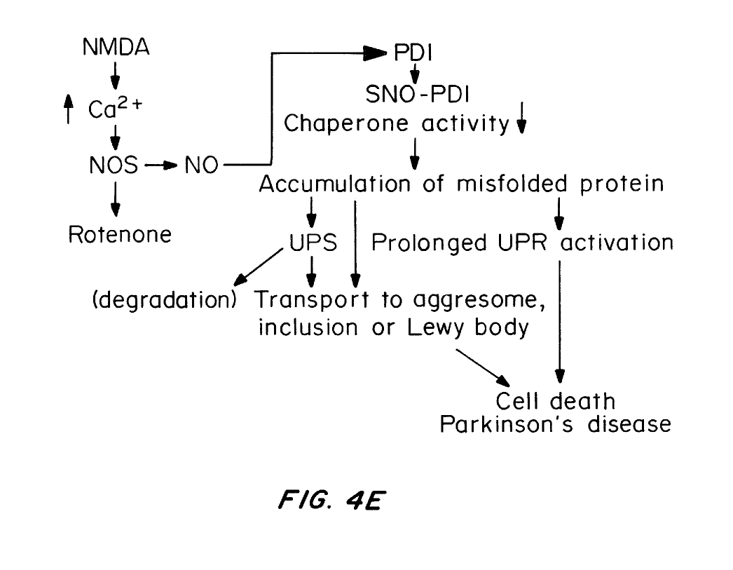

For further clarification of the relationship between the protective function of PDI and its S-nitrosylation, the effect of PDI on neuronal death after ER stress or proteasome inhibition (resulting in the accumulation of polyubiquitinated proteins that cannot be degraded by the proteasome) was investigated. For this purpose SH-SY5Y cells was used because, unlike cortical neurons, they were resistant to direct NO-induced damage under conditions of SNO-PDI formation (See FIG. 1), allowing to reveal the effect of NO on cell death and PDI S-nitrosylation. It was found that cell death precipitated by thapsigargin and tunicamycin (to induce ER stress) or MG132 (to inhibit the proteasome) was largely abrogated by wild-type PDI; however, this protective effect was reversed by exposure to SNOC (See FIG. 4a-c and FIG. 26). Similarly, wild-type PDI ameliorated cell death triggered by overexpression of the Pael receptor, a protein that abnormally accumulates in Parkinson's disease and serves as a potent inducer of the UPR and substrate of the E3 ubiquitin ligase parkin (Imai, Y., Cell, 105, 891-902, (2001); Hara, M. R., Nature Cell Biol., 7, 665-674, (2005)); exposure to SNOC also reversed this protective effect (See FIG. 4d). These results are consistent with the notion that NO impairs the protective role of PDI through S-nitrosylation. From these it can be concluded that cell death in response to proteasome inhibition or ER stress, which contributes to ER dysfunction, UPR activation and protein misfolding, can be attenuated by PDI.

These results show that SNO-PDI forms in brains of patients with PD and AD, neurodegenerative disorders that are characterized by abnormal protein accumulations. Cell models of neurodegeneration produced by exposure to the pesticide rotenone, NO or NMDA also result in the formation of SNO-PDI. S-Nitrosylation of PDI inhibits its activity, allows the accumulation of polyubiquitinated proteins and contributes to neuronal cell death (See FIG. 4e). To determine whether the level of SNO-PDI in neurodegenerative human brain is of pathophysiological significance, the ratio of SNO-PDI (by biotin-switch assay) was calculated to total PDI (from western blotting) and was found that the ratio was similar to that encountered in neuronal cell models manifesting polyubiquitinated proteins and cell death (See FIG. 23). This finding indicates that pathophysiologically relevant amounts of SNO-PDI are present in human brains with PD and AD.

In the absence of nitrosylation, wild-type PDI attenuates abnormal protein accumulation and ubiquitination, including the parkinsonian related protein synphilin-1, and affords neuroprotection. Previous reports have shown the development of ER stress and UPR activation in cellular models of PD (Murakami, T., Ann. Neurol., 55, 439-442, (2004)). In addition, there is increasing evidence that the accumulation of aggregated or misfolded proteins links cellular stress to the pathogenesis of PD (Imai, Y., Cell, 105, 891-902, (2001); Gu, Z., Science, 297, 1186-1190, (2002); Ryu, E. J., J. Neurosci., 22, 10690-10698, (2002)). Other reports have shown that NO can be involved in neurodegeneration by a variety of mechanisms (Conn, K. J., et al. Brain Res., 1022, 164-172, (2004); Sliskovic, I, et al. J. Biol. Chem., 280, 8733-8741, (2005); Nishikawa, A., Biochem. Biophys. Res. Commun., 318, 435-438, (2004); Bonfoco, E., et al. Proc. Natl. Acad. Sci. USA, 92, 7162-7166, (1995); Dawson, V. L., et al. Proc. Natl. Acad. Sci. USA, 88, 6368-6371, (1991); Lipton, S. A., et al. N. Engl. J. Med. 330, 613-622, (1994); Gu, Z., Science, 297, 1186-1190, (2002); Ryu, E. J., J. Neurosci., 22, 10690-10698, (2002)). The data demonstrate a relationship between NO and protein misfolding in degenerative disorders, showing that PDI can be a target of NO after mitochondrial insult in cellular models of PD and in human neurodegenerative diseases. Nitrosative stress resulting in PDI dysfunction therefore provides a mechanistic link between deficits in molecular chaperones, accumulation of misfolded proteins, and neuronal demise in neurodegenerative disorders. The elucidation of this SNO-PDI-mediated pathway that contributes to neuronal injury and apoptosis permits the development of new therapeutic approaches for neurodegenerative diseases and other disorders associated with abnormal protein accumulation and nitrosative stress.

3. Methods

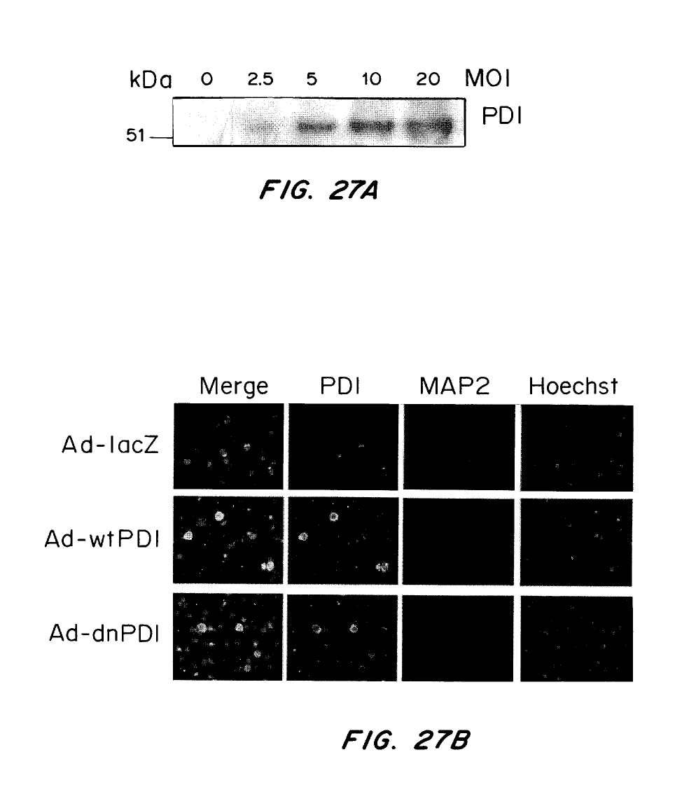

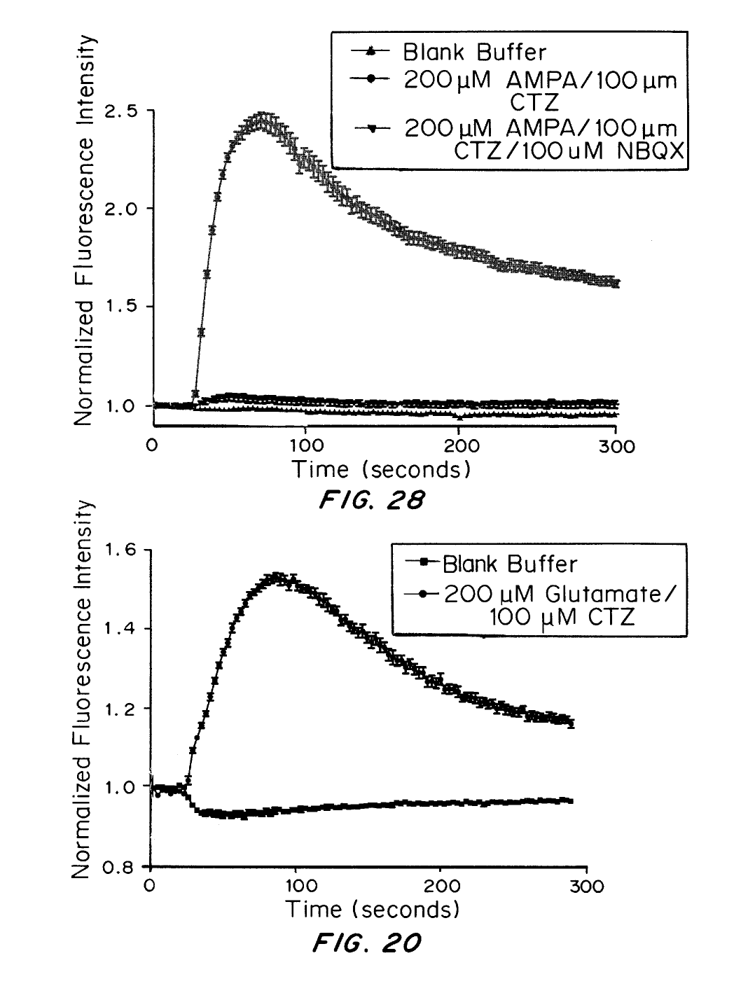

Fluorimetric detection of S-nitrosothiols. S-nitrosothiols were measured by the conversion of DAN (2,3-diaminonaphthalene) to fluorescent compound NAT (2,3-naphthyltriazole), as described (Stamler, J. S., Cell, 78, 931-936, (1994); Sliskovic, I., et al. J. Biol. Chem., 280, 8733-8741, (2005); Gu, Z., Science, 297, 1186-1190, (2002)). NAT was quantified with a FluoroMax-2 spectrofluorometer and DataMax software (Instruments S. A.). Serial NAT dilutions were used to construct a standard curve. Cell injury/death assays. Cerebrocortical neurons or SH-SY5Y cells were transduced with a wild-type or mutated PDI gene or with a Pael receptor expression construct and incubated for 24 h (See FIG. 27). The cells were then treated for 15 h with 5 mM thapsigargin, 10 mgml21 tunicamycin, 0.1 mM MG-132 or diluent in the presence or absence of 100 mM SNOC. Neurotoxicity of the Pael receptor was analyzed as described (Imai, Y., Cell, 105, 891-902, (2001)). Cortical neurons exposed to NMDA were incubated as described previously (Nishikawa, A., Biochem. Biophys. Res. Commun., 318, 435-438, (2004)). Hoechst staining was used to assess morphological changes of apoptotic nuclei. MAP2 staining was used to assess injury or retraction of neuronal processes. Additional details are described elsewhere herein. Expression and purification of recombinant PDI proteins, isolation of PDI complementary DNA and construction of adenoviral vectors, detection of S-nitrosylated proteins with the biotin-switch assay, liquid chromatography-mass spectrometry of PDI, cell culture and transduction by means of adenovirus, PDI enzymatic activity assays, detection of aggregated synphilin-1, immunocytochemistry, XBP-1 mRNA splicing and CHOP mRNA induction, human subjects, and statistics are all described elsewhere herein.

i. Expression and Purification of Recombinant PDI Proteins

Wild-type (wt) or mutated PDI (N, C, and N & C) cDNA was cloned into pGEX4T-1 vector and expressed in BL21 cells according to the manufacture's instructions (Amersham Biosciences). GST-fused PDI was then purified on a column of glutathione Sepharose beads (MicroSpin GST Purification Module). Bound fusion proteins were treated with thrombin to remove GST.

iI. Isolation of PDI cDNA and Construction of Adenoviral Vectors

Full-length rat PDI cDNA and PDI cysteine mutants were isolated as described (Ko, H. S., et al., J. Biol. Chem., 277, 35386-35392, 2002). Additionally, a Myc-tagged PDI construct was produced by PCR, inserting the Myc tag between amino acids E497 and D498, upstream of the KDEL sequence. Each cDNA was subcloned into pShuttle-CMV and then transformed into BJ5183-AD-1 cells that had been pre-transformed with pAdEasy-1. After confirmation that homologous recombination took place in the bacterial cells, the recombinant viral vector was isolated to infect HEK293 cells according to the manufacture's protocol (Stratagene).

iIi. Biotin-Switch Assay for Detection of S-Nitrosylated Proteins

Cell lysates and brain tissue extracts were prepared in HENC or HENT buffers (250 mM Hepes pH 7.5, 1 mM EDTA, 0.1 mM neocuproine, 0.4% CHAPS or 1% Triton X-100). A range of protein concentrations were tested in these assays, but typically 1 mg of cell lysate and up to 2 mg of tissue extract were used. Blocking buffer (2.5% SDS, 20 mM methyl methane thiosulphonate [MMTS] in HEN buffer) was mixed with the samples and incubated for 30 min at 50° C. to block free thiol groups. After removing excess MMTS by acetone precipitation, nitrosothiols were then reduced to thiols with 1 mM ascorbate. The newly formed thiols were then linked with the sulphhydryl-specific biotinylating reagent N-[6-(biotinamido)hexyl]-3′-(2′-pyridyldithio)propionamide (Biotin-HPDP). We then pulled down the biotinylated proteins with Streptavidin-agarose beads and performed Western blot analysis to detect the amount of PDI remaining in the samples (Gu, Z., Science, 297, 1186-1190, (2002); Yao, D., Proc. Natl. Acad. Sci. USA, 101, 10810-10814, (2004)). The input for these blots was typically 2 μg of cell lysate or 100 μg of tissue extract loaded in each lane.

iv. Liquid Chromatography-Mass Spectrometry (LC-MS) of PDI

Mass spectra were obtained after in-solution trypsin digestion of rat recombinant PDI using an LC Packings nano-LC system and a quadrupole time-of-flight mass spectrometer (Q-TOF API US, Micromass/Waters Corp.) using previously described methods Gu, Z., Science, 297, 1186-1190, (2002); Sliskovic, I, et al. J. Biol. Chem., 280, 8733-8741, (2005); Nadal M. S. et al. Neuron 37, 449-461 (2003)). Briefly, recombinant PDI was exposed to a range of concentrations of the NO donor SNOC in vitro for 30 min at room temperature. Remaining free thiols in PDI were blocked with 20 mM methyl methane thiosulphonate (MMTS) in HEN buffer at 50° C. for 30 min, followed by in-solution digestion with trypsin at 37° C. for 24 h. The analytical column was a PepMap C18 (LC Packings) with dimensions of 75 μm i.d.×15 cm. Mobile phase A consisted of 2% acetonitrile/0.1% formic acid, and mobile phase B was 80% acetonitrile/0.1% formic acid. For mobile phase B, the gradient was 2%˜90% over 45 min, followed by 90% for 10 min at a flow rate of ˜200 nl/min. MS/MS data were processed with MassLynx 4.0, ProteinLynx Global Server 2.01 and MaxEnt 3 (Micromass/Waters Corp). Modified cysteine residues of PDI were identified from the resulting .pk1 files using Mascot (Matrix Science, UK). These results were further validated by manual interpretation of the deconvoluted MS/MS spectra with assistance of the PepSeq program (Micromass/Waters Corp).

v. Cell Culture and Transduction Using Adenovirus

SH-SY5Y, HEK293T, or HEK293 cells stably expressing nNOS were grown in DMEM containing 10% FCS in a 5% CO₂/balance air atmosphere (Bredt, D. S. et al. Nature 351, 714-718 (1991); Choi, Y. B. et al. Nature Neurosci. 3, 15-21 (2000)). Cells were transiently infected with adenovirus at a multiplicity of infection of 10 (data shown in FIG. 27). Primary cortical cultures were prepared as described (Budd, S. L., et al. Proc. Natl. Acad. Sci. USA 97, 6161-6166 (2000); Okamoto, S. i. et al. Proc. Natl. Acad. Sci. USA 99, 3974-3979 (2002)), exposed to NMDA (50 μM plus 5 μM glycine for 20 min) in Earle's balanced salt solution, and then rinsed and replaced in the original conditioned medium. Neurons were subsequently assessed for survival.

vi. Assay for PDI Enzymatic Activity

Chaperone activity of PDI was measured by analyzing the renaturation (refolding) of denatured rhodanese in the presence of 1 μM PDI or its mutant form (Vuori, K., et al. J. Biol. Chem, 267, 7211-7214, (1992); Horibe, T. et al. J. Biol. Chem. 279, 4604-4611 (2004); Martin, J. et al. Nature 352, 36-42 (1991)). Rhodanese was denatured by guanidinium and then diluted in buffer (30 mM Tris-HCl, 50 mM KCl, pH 7.2) with or without 1 μM recombinant PDI. The aggregation of denatured rhodanese was monitored by the increase in absorbance at 320 nm. The value of denatured rhodanese after 20 min in the absence of PDI was set to 100%.

Isomerase activity of PDI was determined by measuring RNase A activity regenerated from scrambled RNase A¹⁰ (web site bio.takara.co.jp/bio_en/Catalog_d.asp?C_ID=C0606). One unit of PDI was defined as the amount required to recover one RNase A unit from reduced bovine RNase A at 25° C. in 15 minutes at pH 7.5. One RNase A unit was defined as the amount required for hydrolysis of cCMP (cytidine 2′,3′-cyclic monophosphate) that results in an increase of 0.001 absorbance unit at 284 nm per minute at 25° C., pH 7.5.

vii. Detection of Aggregated Synphilin-1

SH-SY5Y cells were transfected with GFP-fused synphilin-1 (GFP-Synp) and/or PDI (1 μg each) using Lipofectamine 2000 reagent in Opti MEM. The cells were placed in fresh medium (DMEM) containing fetal calf serum 5 h after transfection, exposed to 100 μM SNOC or control solution, and then incubated for another 19 h. The cells were fixed with 1% glutaraldehyde for 10 min, rinsed three times with PBS, and stained with Hoechst dye for 10 min. Stained cells were observed under epifluorescence deconvolution microscopy.

viii. Immunocytochemistry

Cells were fixed with 4% paraformaldehyde (PFA) for 10 min at room temperature and then washed three times in PBS, permeabilized with 0.5% Triton X-100 for 5 min, and washed three times. Non-specific antibody binding was minimized by incubation in blocking solution (10% goat serum in PBS) for 1 h at 37° C. Cells were incubated with antibodies to poly-Ub protein (BIOMOL) or MAP2 (Sigma) for 12 h at 4° C. After washing, the cells were incubated with anti-mouse or anti-rabbit antibodies conjugated with Alexa 488/594 for 1 h at 37° C. and then incubated in Hoechst 33342 dye (1 μg/ml) to fluorescently stain nuclei and allow assessment of nuclear morphology to determine apoptosis.

ix. XBP-1 mRNA Splicing and CHOP mRNA Induction

Each sample of total RNA was isolated using TR1 reagent (Sigma). To detect whether IRE1α cleaved the 26-nt segment from endogenous XBP-1 mRNA, PCR was used to amplify a 600-bp cDNA encompassing nucleotides 571-1144 in the mRNA sequence. Digestion of the unprocessed 600-bp cDNA with the restriction enzyme Pst I normally yields two fragments of about 300 bp on electrophoresis. However, cleavage by active IRE1α of a 26-nt segment in the XBP-1 mRNA yields a 574-bp amplification product and removes the Pst I restriction site (Ellgaard, L., et al. Science, 286, 1882-1888, (1999); Kaufman, R. J., Genes Dev. 13, 1211-1233, (1999); Calfon, M. et al. Nature 415, 92-96 (2002); Yoshida, H., et al. Cell 107, 881-891 (2001)). Endogenous XBP-1 mRNA was amplified by RT-PCR using following primers: sense 5′-AAA CAG AGT AGC AGC GCA GAC TGC-3′ (SEQ ID NO:2); antisense 5′-GGA TCT CTA AAA CTA GAG GCT TGG TG-3′ (SEQ ID NO:3). The PCR product was then digested with Pst I for 2 h at 37° C. and detected on 2% agarose gels.

For amplification of CHOP and GAPDH mRNA, the primers were as follows: CHOP sense; 5′-TCT GCC TTT CGC CTT TGA GAC-3′ (SEQ ID NO:4); CHOP antisense; 5′-CCC GGG CTG CGC ACT GAC CAC-3′ (SEQ ID NO:5); GAPDH sense; 5′-AAA CCC ATC ACC ATC TTC CAG-3′(SEQ ID NO:6); GAPDH antisense; 5′-AGG GGC CAT CCA CAG TCT TCT-3′ (SEQ ID NO:7). The primer sets for CHOP and GAPDH produced PCR products with a size of 326 and 361 bp, respectively.

x. Statistics

All data are expressed as mean±s.e.m. Statistical evaluation was performed by analysis of variance (ANOVA) followed by a post-hoc Scheffe test. Each experiment was performed ≧3 times in triplicate.

### B. Example 2

### Pathologically Activated Therapeutics for Neuroprotection

Although many factors, including absorption, distribution, metabolism, excretion (ADME) and pharmacokinetics, complicate drug development in general, brain function is particularly susceptible to disruption because many of the targets for drug action exert normal physiological actions in unaffected parts of the brain. Strong inhibition of these targets can block normal as well as abnormal activity.

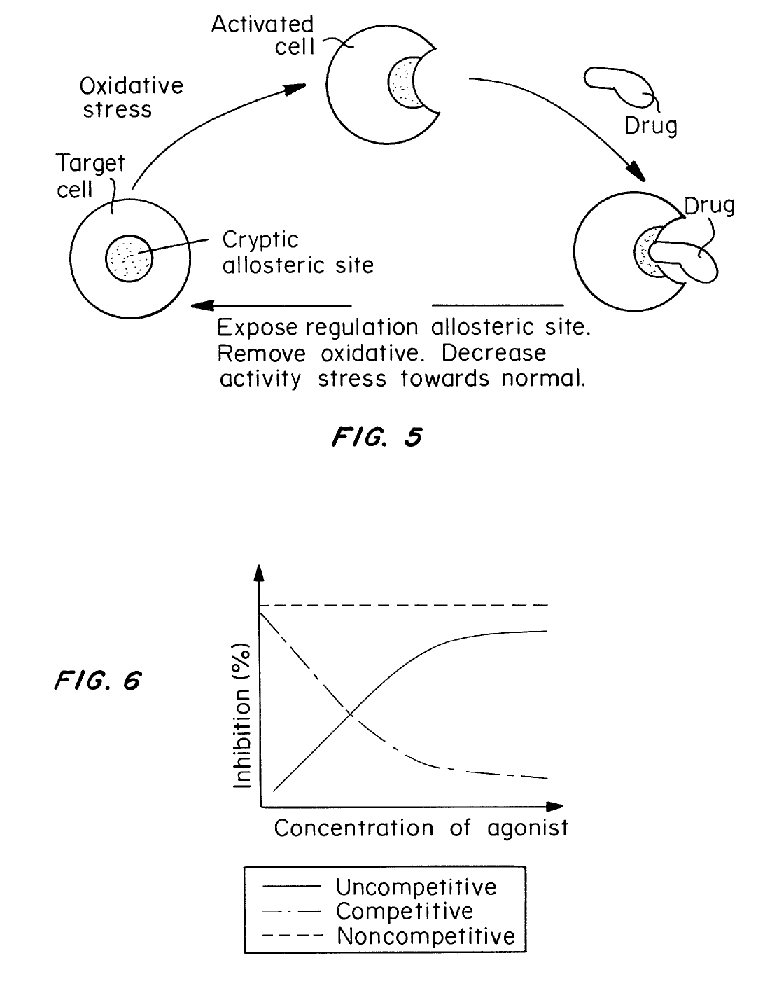

Described herein are strategies for the development of neuroprotective drugs that are clinically well tolerated. These strategies are based on the principle that drugs should interact with their target primarily during states of pathological activation but not interfere with the target if it functions normally. Such drugs preferably should therefore exhibit little inhibition of normal physiological function. Drugs developed using these strategies can be referred to as pathologically activated therapeutic (PAT) drugs (Lipton, S. A., Nature 428, 473 (2004); Lipton, S. A. et al., Cell Death Differ. 11, 18-20 (2004). The design of PAT drugs is based on mechanistic insights into their mode of action. These drugs can home in on and antagonize receptor channels, enzymes or other molecules that are excessively activated under pathological conditions. For instance, an allosteric modulator site on a drug target that remains cryptic under normal conditions may become exposed during excessive activation in pathological circumstances. This allows the PAT drug to inhibit the unwanted activity of the target, whereas it has little or no effect on the target's normal physiological activity (see FIG. 5).

Some older drugs are also known to act selectively in pathological tissue. For example, lidocaine, dilantin and dihydropyridines act, at least in part, by preferentially blocking Na⁺ channels (in the case of lidocaine and dilantin) or Ca²⁺ channels (in the case of dihydropyridines) on cells that are pathologically activated. In the case of lidocaine, this results in its local anaesthetic effect (Butterworth, J. et al. Anesthesiology 72, 711-734 (1990). Dihydropyridines selectively blocks L-type Ca²⁺ channels that are inactivated by depolarization (Bean, B. P. Proc. Natl. Acad. Sci. USA 81, 6388-6392 (1984); Bean, B. P. Nature 447, 1059-1060 (2007); Chan, C. S. et al. ‘Nature 447, 1081-1086 (2007)). As depolarization occurs in cells that are injured, these cells are preferentially affected by dihydropyridines.

Disclosed are PAT drugs with improved strategies, including uncompetitive antagonists; S-nitrosylation targeted to pathological areas of the brain; upregulation of drug targets by the pathological process; target-induced drug activation (Bean, B. P. Nature 447, 1059-1060 (2007); Chan, C. S. et al. Nature 447, 1081-1086 (2007)); and activation of pro-drugs by abnormal redox states of the tissue.

### C. Example 3

### Paradigm Shift in Neuroprotection by NMDA Receptor Blockade: Memantine and Beyond

The molecular basis for memantine efficacy in neurological diseases that are mediated, at least in part, by overactivation of NMDARs, producing excessive Ca²⁺ influx through the receptor's associated ion channel and consequent free-radical formation.

Dementia is a major cause of disability and death worldwide. Alzheimer's disease, the leading cause of dementia, ranks fourth in mortality in the US. The prevalence of vascular dementia (multi-infarct dementia) is not far behind Alzheimer's disease. Often elderly patients display both types of disease. Economists claim that the ageing population will consume the entire gross national product of western countries by 2050 for treatment of dementia. Excitotoxic (glutamate-related) neuronal cell injury and death is thought to contribute to this and virtually every other major neurodegenerative disorder (Lipton, S. A. N. Engl. J. Med. 330, 613-622 (1994); Lipton, S. A. et al. Cell Calcium 23, 165-171 (1998); Choi, D. Neuron 1, 623-634 (1988); Meldrum, B. et al. Trends Pharmacol. Sci. 11, 379-387 (1990); Rothman, S. Trends Neurosci. 10, 299-302 (1987)). Oligodendrocytes, providing the myelin sheath, can also be injured by this mechanism, indicating that excitotoxic injury could have a role in white matter (glial) as well as grey matter (neuronal) disorders. Excitotoxicity occurs in part because of overactivation of N-methyl-D-aspartate (NMDA)-sensitive glutamate receptors, permitting excessive Ca²⁺ influx through the receptor's associated ion channel.

Importantly, however, normal glutamate receptor activity mediates, in large measure, physiological excitatory synaptic transmission in the brain and is therefore crucial for the normal functioning of the nervous system. There are three classes of glutamate-gated ion (or ionotropic) channels, known as AMPA receptors, kainate receptors and NMDA receptors (NMDARs). Among these, the ion channels coupled to classical NMDARs are generally the most permeable to Ca²⁺. Excessive activation of the NMDAR in particular therefore leads to increased intracellular Ca²⁺ and the consequent production of damaging free radicals and activation of proteolytic processes that contribute to cell injury or death (Lipton, S. A., N. Engl. J. Med. 330, 613-622 (1994); Lipton, S. A. et al., Cell Calcium 23, 165-171 (1998); Choi, D., Neuron 1, 623-634 (1988); Meldrum, B. et al., Trends Pharmacol. Sci. 11, 379-387 (1990); Rothman, S. Trends Neurosci. 10, 299-302 (1987)). With the disruption of energy metabolism during acute and chronic neurodegenerative disorders, glutamate is not cleared properly and can even be inappropriately released. Moreover, energetically compromised neurons become depolarized (that is, more positively charged) because in the absence of energy they cannot maintain their ionic homeostasis; this depolarization relieves normal block of NMDAR coupled channels by Mg because the relatively positive charge in the cell repels positively charged Mg²⁺ from the channel pore. In many neurodegenerative diseases, therefore, excessive stimulation of glutamate receptors is thought to occur even in the absence of excessive glutamate levels (Zeevalk, G. et al., J. Neurochem. 59, 1211-1220 (1992). Disorders including Alzheimer's disease, Parkinson's disease, Huntington's disease, HIV-associated dementia (or HIV-associated neurocognitive disorders), multiple sclerosis, amyotrophic lateral sclerosis (ALS), neuropathic pain and glaucoma are caused by different mechanisms but might share a final common pathway to neuronal injury that is due to the overstimulation of glutamate receptors, especially of the NMDA subtypel. Acute disorders, such as stroke, central nervous system (CNS) trauma and seizures, also manifest a component of excitotoxicity. NMDAR antagonists can be of therapeutic benefit in a number of neurological disorders.

NMDARs are made up of different subunits: NR1 (whose presence is mandatory), NR2A-D, and, in some cases, NR3A or NR3B subunits. The receptor is probably composed of a tetramer of these subunits. The subunit composition determines the pharmacology and other parameters of the receptor-ion channel complex. Alternative splicing of some subunits, such as NR1, further contributes to the pharmacological properties of the receptor. The subunits are differentially expressed both regionally in the brain and temporally during development. Additionally, the NR2B subunit predominates in extrasynaptic rather than synaptic NMDARs, and it is thought that excessive stimulation of extrasynaptic (NR2B predominant) receptors contribute to neurotoxicity, whereas physiological synaptic activity (predominantly containing NR2A>NR2B subunits) might be neuroprotective (Hardingham, G. et al., Nature Neurosci. 5, 405-414 (2002). For this reason some authorities have indicated developing antagonists that are selective for particular subunits, such as NR2B, which is predominantly present in the forebrain (Kemp, J. et al. Nat. Neurosci. 5 (Suppl.), 1039-1042 (2002)).

1. Relationship of Vascular Dementia and Excitotoxic Damage

The glutamate content of whole brain is approximately 10 mM. Because of the activity of glutamate transporters, predominantly on astrocytes, most of this glutamate is intracellular. The extracellular glutamate concentration in brain has been estimated to be approximately 0.6 μm. The sensitivity to excitotoxicity of cultured cortical neurons isolated away from astrocytes is approximately 2-5 μM glutamate. Therefore, the ambient concentrations of glutamate are close to those that can cause neuronal death, and it is important that extracellular glutamate concentration and its compartmentalization be exquisitely controlled to prevent excitotoxicity. On the other hand, with 10 mM glutamate present in cells, the potential for disaster is very great.

Extracellular glutamate levels have been shown to rise in the face of hypoxic-ischaemic insults as seen in vascular dementia. There will probably prove to be several mechanisms for the excess accumulation of glutamate, even in a single disorder such as ischemial. Energy failure might cause abnormal accumulations of glutamate either by impairment of uptake (into neurons and especially astrocytes) mediated via glutamate transporters or by reversal of the direction of transport. This series of events would be followed by injury to some neurons and abnormal potentiation of glutamate release from others. With glutamate release from injured neurons, and excess physiological release from otherwise intact neighboring neurons, the process might then develop a self-propagating character that extends the area of neuronal damage.

Deprivation of oxygen and glucose—for example, during ischaemia—cause a decrement in the production of high-energy phosphate compounds and ‘energy failure.’ However, short-term energy failure per se is not particularly toxic to neurons. What does make energy failure highly neurotoxic is the activation of glutamate receptor-dependent mechanisms. If these are blocked by suitable glutamate antagonists, then neurons can survive a period of oxygen and metabolic-substrate deprivation (Lipton, S. A. et al., N. Engl. J. Med. 330, 613-622 (1994); Zurakowski, D. et al., Vision Res. 38, 1489-1494 (1998)).

2. Clinically Tolerated NMDAR Antagonists

Excitotoxicity represents a particularly attractive target for neuroprotective efforts because it is implicated in the pathophysiology of a wide variety of acute and chronic neurodegenerative disorders1. The challenge facing those trying to devise strategies for combating excitotoxicity is that the same processes that, in excess, lead to excitotoxic cell death are, at lower levels, absolutely crucial for normal neuronal function. Until recently, all drugs that showed promise as inhibitors of excitotoxicity also blocked normal excitatory synaptic activity and consequently had severe and unacceptable side effects. As a result, prior drugs lacked clinical tolerability, representing the major (but not the only) factor involved in the failure of these drugs in clinical trials for stroke, traumatic brain injury and Huntington's disease Lees, K. R. et al. Lancet 355, 1949-1954 (2000); Sacco, R. L. et al. JAMA 285, 1719-1728 (2001); Kemp, J. et al. in Handbook of Experimental Pharmacology (eds. Jonas, P. & Monyer, H.) 495-527 (Springer, Berlin, 1999)).

However, a mechanism has been discovered through which an old drug, memantine, was not only capable of blocking excitotoxic cell death (Seif el Nasr. et al. Eur. J Pharmacol. 185, 19-24 (1990)), but, most importantly, of doing it in a clinically tolerated manner (Lipton, S. Trends Neurosci 16, 527-532 (1993); Chen, H. J. Physiol. (Lond.) 499, 27-46 (1997); Chen, H.-S. V. et al. J. Neurosci. 12, 4427-4436 (1992); Chen, H.-S. V. et al. Neuroscience 86, 1121-1132 (1998)). Memantine was recently approved by the European Union and the US FDA for the treatment of moderate-to-severe Alzheimer's disease, and might show efficacy for more mild Alzheimer's disease and vascular dementia (Reisberg, B. et al. N. Engl. J. Med. 348, 1333-1341 (2003); Orgogozo, J. et al. Stroke 33, 1834-1839 (2002)). Prior to this time, cholinergic drugs had been approved for the treatment of Alzheimer's disease, but they offered only symptomatic relief (slowing of cognitive decline), whereas memantine is believed to be the first neuroprotective drug to achieve clinical approval. Memantine is also being developed as a treatment for other neurodegenerative disorders, including HIV-associated dementia, neuropathic pain, glaucoma, depression, Huntington's disease, ALS and movement disorders, among others (Lipton, S. et al. N. Engl. J. Med. 330, 613-622 (1994)).

Memantine can be used to illustrate strategies for developing neuroprotective drugs.

3. Excitotoxic Damage and Alzheimer's Disease

There are several links between excitotoxic damage and the primary insults of Alzheimer's disease, which, based on rare familial forms of the disease, are thought to involve toxicity from misfolded mutant proteins (Rogawski, M. A. et al. CNS Drug Rev. 9, 275-308 (2003). These proteins include soluble oligomers of β-amyloid peptide (A(3, particularly Aβ₁₋₄₂) and hyperphosphorylated tau proteins (Selkoe, D. J. Alzheimer's disease: genes, proteins, and therapy. Physiol. Rev. 81, 741-766 (2001). For example, oxidative stress and increased intracellular Ca²⁺ generated in response to Aβhave been reported to enhance glutamate-mediated neurotoxicity in vitro. Additional experiments indicate that Aβcan increase N-methyl-D-aspartate (NMDA) responses and therefore excitotoxicity (Wu, P. H., et al. Neurochem. Res. 9, 1019-1031). Another link comes from recent evidence that glutamate transporters are downregulated in Alzheimer's disease and that Aβcan either directly or indirectly inhibit glutamate reuptake or even enhance its release. Excessive NMDAR activity has also been reported to increase the hyperphosphorylation of tau, which contributes to neurofibrillary tangles. The NMDAR antagonist memantine has been found to offer protection from intrahippocampal injection of Aβ. Moreover, memantine improved performance on behavioural tests (T-maze and Morris water maze) in a transgenic mouse model of Alzheimer's disease consisting of a mutant form of presenilin-1 and amyloid precursor protein. Finally, memantine was recently found to reduce tau hyperphosphorylation, at least in culture.

4. Excitotoxicity in Neurological Disorders

Clinical relevance of basic concepts. The capacity of the nervous system to rapidly convey sensory information and motor commands from one part of the body to another, and to form thoughts and memories, is largely dependent on a single powerful excitatory neurotransmitter: glutamate. Most neurons and glia contain high concentrations of glutamate (˜10 mM) (Lipton, S. A. et al., N. Engl. J. Med. 330, 613-622 (1994)). After sequestration inside synaptic vesicles, glutamate is released for very brief amounts of time (milliseconds) to communicate with other neurons via synaptic endings. Because glutamate is so powerful, however, its presence in excessive amounts, or for excessive periods of time, can literally, excite cells to death. This phenomenon was first documented by Lucas and Newhouse (Lucas, D. et al., Arch. Ophathalmol. 58, 193-201 (1957)); Olney later coined the term ‘excitotoxicity’ to describe the event (Olney, J., Neuropathol. Exp. Neurol. 28, 455-474 (1969); Olney, J., Nature 227, 609-611 (1970)).

A large variety of insults can lead to the excessive release of glutamate or excessive stimulation of glutamate receptors within the nervous system, and thereby trigger excitotoxic injury or even cell death. When the nervous system suffers a severe mechanical insult, as in head or spinal cord injury, large amounts of glutamate are released from injured cells. These high levels of glutamate reach thousands of nearby cells that survived the original trauma, causing them to depolarize, swell, lyse and die by necrosis. The lysed cells release more glutamate, leading to a cascade of autodestructive events and progressive cell death that can continue for hours or even days after the original injury. A similar phenomenon occurs in stroke: the ischaemic event deprives many neurons of the energy they need to maintain ionic homeostasis, causing the intracellular buildup of cations with consequent depolarization, osmotic swelling, lysis and death, which propagates the same type of autodestructive events that are seen in traumatic injury (Lipton, S. A., Curr. Opin. Neurol. Neurosurg. 6, 588-596 (1993)). This acute form of cell death occurs by a necrotic-like mechanism, although a slower component leading to an apoptotic-like death can also be present, as well as a continuum of events somewhere between the two (Ankarcrona, M. et al., Neuron 15, 961-973 (1995); Bonfoco, E., Proc. Natl. Acad. Sci. USA 92, 7162-7166 (1995)).

In fact, less fulminant, subtler forms of excitotoxicity are implicated in a variety of slowly progressing neurodegenerative disorders, as well as in the penumbra of acute stroke damage. In chronic disorders, it is proposed that exposure to modest glutamate receptor hyperactivity for longer periods of time than occur during normal neurotransmission trigger cellular processes that eventually lead to neuronal injury or apoptotic-like cell death (Lipton, S. A. et al., Cell Calcium 23, 165-171 (1998); Bonfoco, E., et al. Proc. Natl. Acad. Sci. USA 92, 7162-7166 (1995); Dreyer, E. et al. NeuroReport 6, 942-944 (1995); Quigley, H. A. et al. Invest. Ophthalmol. Vis. Sci. 36, 774-786 (1995); Vorwerk, C. K. et al., Invest. Ophthalmol. Vis. Sci. 37, 1618-1624 (1996); Dreyer, E. et al., Clin. Neurosci. 4, 270-273 (1997); Dreyer, E. et al., JAMA 281, 306-308 (1999); Naskar, R. et al. Semin. Ophthalmol. 14, 152-158 (1999)). Importantly, the initial manifestation of these more subtle insults produces synaptic, dendritic and possibly axonal damage in the early stages of disease (Berliocchi, L. et al. J. Cell Biol. 168, 607-618 (2005); Garden, G. A. et al., J. Neurosci. 22, 4015-4024 (2002)). These processes precede cell death and might be reversible, and are therefore of considerable therapeutic interest. A key concept in this form of neuronal damage is that elevation in extracellular glutamate is not necessary to invoke an excitotoxic mechanism if NMDAR activity is increased because of relief from normal Mg²⁺ blockade (Zeevalk, G. et al., J. Neurochem. 59, 1211-1220 (1992).

i. Mechanism of NMDA Receptor Contribution to Excitotoxic Disorders.

During normal neurotransmission, the NMDA channel is activated for only brief (millisecond) periods of time due to relief of Mg²⁺ blockade, which occurs after cation influx into the neuron via AMPA sensitive glutamate receptor channels. In some areas of the brain, normal NMDAR activity is important for the induction of long-term potentiation (LTP), which is thought to be a cellular-electrophysiological correlate of learning and memory formation. Moreover, the reticular activating system in the brainstem, an area responsible for wakefulness and attention, bears many NMDARs, which, if not functioning normally, results in drowsiness and even coma. It is therefore very important to preserve physiological NMDAR activity in order to preserve these normal functions and avoid intolerable clinical side effects.

Under pathological conditions, however, over activation of the NMDAR causes an excessive Ca²⁺ influx into the nerve cell, leading to cellular damage and death.

In chronic neurodegenerative disorders signaling cascades lead to synaptic damage and eventually apoptotic-like cell death (see FIG. 12). These injurious processes include Ca²⁺ overload of mitochondria, resulting in oxygen free-radical formation, activation of caspases and release of apoptosis-inducing factor; Ca²⁺-dependent activation of neuronal nitric oxide synthase (nNOS), leading to increased nitric oxide (NO) production and the formation of toxic peroxynitrite (ONOO—) and S-nitrosylated GAPDH; and stimulation of p38 mitogenactivated protein kinase (p38 MAPK), which activates transcription factors that can go into the nucleus to influence neuronal injury and apoptosis (Bonfoco, E. et al., Proc. Natl. Acad. Sci. USA 92, 7162-7166 (1995); Dawson, V. L., et al. Proc. Natl. Acad. Sci. USA 88, 6368-6371 (1991); Dawson, V. L., et al. J. Neurosci 13, 2651-2661 (1993); Lipton, S. A. et al., Nature 364, 626-632 (1993); Tenneti, L., et al. J. Neurochem. 71, 946-959 (1998); Yun, H.-Y., et al. Proc. Natl. Acad. Sci. USA 95, 5773-5778 (1998); Budd, S. et al., Proc. Natl. Acad. Sci. USA 97, 6161-6166 (2000); Okamoto, S. i. et al., Proc. Natl. Acad. Sci. USA 99, 3974-3979 (2002); Wang, H. et al., J. Neurosci. 24, 10963-10973 (2004); Hara, M. et al., Nature Cell. Biol. 7, 665-674 (2005)).

iI. Pharmacological and Covalent Binding Sites on the NMDAR.

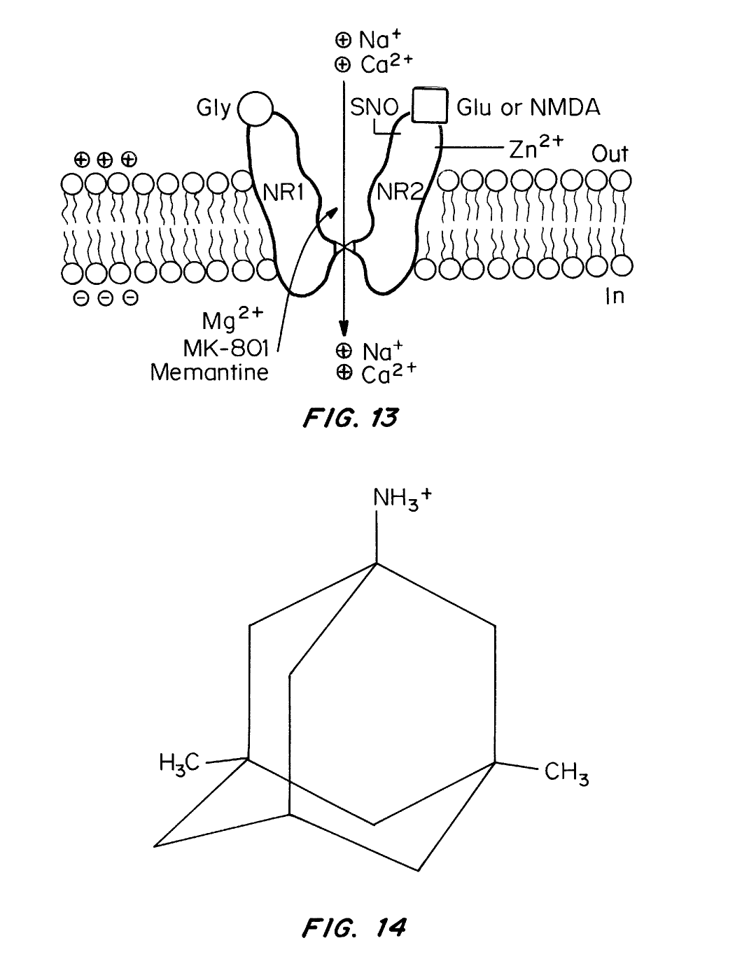

The NR2 family of NMDAR subunits contains a binding site for glutamate, the endogenous agonist, whereas the NR1 subunit binds glycine42, which is required as a co-agonist for receptor activation (see FIG. 13). Interestingly, d-serine is an endogenous substance that is made predominantly in astrocytes, and is at least, if not more, potent than glycine itself at the glycine site of classical NMDARs (composed of NR1/NR2 subunits). Although levels of glycine are tenfold higher than d-serine, recent reports indicate that endogenous D-serine and not glycine is the dominant endogenous co-agonist for NMDAR-mediated neurotoxicity (Shleper, M., et al. J. Neurosci. 25, 9413-9417 (2005); Mothet, J. P. et al. Proc. Natl. Acad. Sci. USA 97, 4926-4931 (2000); Wolosker, H., et al. Proc. Natl. Acad. Sci. USA 96, 13409-13414 (1999)).

Note that NMDA is not an endogenous substance in the body, but an experimental tool that is highly selective for this subtype of glutamate binding site and which therefore became the source of the receptor's name. When glutamate and glycine bind and the cell is depolarized to remove Mg²⁺ block, then the NMDAR channel opens with consequent influx of Ca²⁺ and Na²⁺ into the cell, the amount of which can be altered by higher levels of agonists and by substances binding to one of the modulatory sites on the receptor. The two modulatory sites that are most relevant to pathologically-activated drugs are the magnesium site, which is located within the ion channel, and S-nitrosylation sites, which are located towards the amino-terminus (and therefore extracellular region) of the receptor. S-Nitrosylation represents a covalent chemical reaction in which NO is transferred to a thiol or sulphydryl group (—SH) of a crucial cysteine residue. This reaction modulates protein function, in this case decreasing excessive channel activity associated with stimulation of the NMDAR. These sites are targets for therapeutic intervention to block excitotoxicity. Other modulatory sites also exist on the NMDAR and can be of therapeutic value. These include binding sites for Zn²⁺ and polyamines, as well as a pH-sensitive site (Lipton, S. A. et al., Cysteine regulation of protein function—as exemplified by NMDA-receptor modulation. Trends Neurosci. 25, 474-480 (2002)). Additionally, other redox-sensitive cysteine residues that are not susceptible to S-nitrosylation can nonetheless inhibit channel activity by virtue of their redox-sensitive capacity to form disulphide bonds (Lipton, S. A. et al. Cysteine regulation of protein function—as exemplified by NMDA-receptor modulation. Trends Neurosci. 25, 474-480 (2002)).

5. Concepts for Clinically Tolerated Antagonists

Competitive antagonists result in intolerable side effects. To be clinically acceptable, an anti-excitotoxic therapy should block excessive activation of the NMDAR while leaving normal function relatively intact in order to avoid side effects. Drugs that simply compete with glutamate or glycine at the agonist-binding sites block normal function and therefore do not meet this requirement, and have consequently failed in clinical trials to date because of side effects (such as drowsiness, hallucinations and even coma) (Lipton, S. A. et al., N. Engl. J. Med. 330, 613-622 (1994); Kemp, J. Nat. Neurosci. 5 (Suppl.), 1039-1042 (2002); Lipton, S. A Trends Neurosci 16, 527-532 (1993); Koroshetz, W. J et al., Trends Pharmacol. Sci. 17, 227-233 (1996); Hickenbottom, S. Semin. Neurol. 18, 485-492 (1998); Lutsep, H. et al. Drugs R & D 1, 3-8 (1999); Rogawski, M., Amino Acids 19, 133-149 (2000); Palmer, G. Curr. Drug Targets 2, 241-271 (2001)). What are the reasons for this? Competitive antagonists compete one for one with the agonist (glutamate or glycine) and will therefore block healthy areas of the brain (where lower, more physiological levels of these agonists exist) before they can affect pathological areas (where higher levels of agonist accumulate or higher levels of receptor activation are present). In fact, such drugs would preferentially block normal activity and would probably be displaced from the receptor by the high local concentrations of glutamate or glycine for prolonged periods that can exist under excitotoxic conditions.

i. Uncompetitive, Open-Channel Block: Importance of Off-Rate.

One mechanism that can result in blockade of excessive NMDAR activity while relatively sparing normal physiological activity involves blockade of the ion channel when it is excessively open (termed open-channel block). As a helpful analogy, the NMDAR can be thought of as a television set. The agonist sites are similar to the ‘on/off’ switch of the television. Drugs that block here cut off all normal NMDAR function and result in clinically unacceptable side effects. Instead, we need to find the equivalent of the volume control (or, in biophysical terms, the gain) of the NMDAR. Then, during excessive Ca²⁺ flux through the NMDAR-associated ion channel, the ‘volume’ of ion flow would be turned down towards normal. A blocker that acts at the Mg²⁺ site within the channel could act in such a manner. However, in the case of Mg²⁺ itself, the block is too ephemeral, creating a so-called ‘flickery block,’ and the cell continues to depolarize until the Mg²⁺ block is totally relieved. As a result, in most cases Mg²⁺ does not effectively block excessive Ca²⁺ influx to the degree needed to prevent neurotoxicity. If, on the other hand, a channel blocker binds too tightly or works too well at low levels of receptor activation, it will block normal as well as excessive activation and be clinically unacceptable. Using the television set analogy, turning the volume all the way down is as bad as turning the ‘on/off’ switch to the off position in terms of blocking normal function of the television. This is the case with MK-801; it is a very good excitotoxicity blocker, but because its ‘dwell time’ in the ion channel is so long (reflecting its slow ‘off-rate’) due to its high affinity for the Mg²⁺ site, it also blocks crucial normal functions. A human taking a neuroprotective dose of MK-801 would not only become drowsy, but would lapse into a coma. Drugs with slightly shorter but still excessive dwell times (off-rates) make patients hallucinate (for example, phencyclidine, also known as Angel Dust), or so drowsy that they can serve as anesthetics (for example, ketamine).

A clinically tolerated NMDAR antagonist should not make a patient drowsy, hallucinate or comatose, and in fact should spare normal neurotransmission while blocking the ravages of excessive NMDAR activation. In fact, one type of drug that can accomplish this feat and block preferentially higher (pathological) levels of glutamate over normal (physiological) levels is an ‘uncompetitive’ antagonist. An uncompetitive antagonist can be distinguished from a noncompetitive antagonist (which acts allosterically at a noncompetitive site—that is, at a site other than the agonist-binding site). An uncompetitive antagonist is defined as an inhibitor whose action is contingent on prior activation of the receptor by the agonist. This means that the same amount of antagonist blocks higher concentrations of agonist to a greater degree than lower concentrations of agonist. Some open channel blockers function as pure uncompetitive antagonists, depending on their exact properties of interaction with the ion channel. This uncompetitive mechanism of action coupled with a slower off-rate from the channel than Mg²⁺ but a substantially faster off-rate than MK-801 would yield a drug that preferentially blocks NMDAR-operated channels when they are excessively open while relatively sparing normal neurotransmission. As elaborated below, experiments indicated that memantine is such a drug (Lipton, S. A. et al., N. Engl. J. Med. 330, 613-622 (1994); Lipton, S. A., Trends Neurosci 16, 527-532 (1993); Chen, H.-S. V. and Lipton, S. A., J. Physiol. (Lond.) 499, 27-46 (1997); Chen, H.-S. V. et al., J. Neurosci. 12, 4427-4436 (1992); Chen, H.-S. V. et al., Neuroscience 86, 1121-1132 (1998)).

An important discovery that led to the testing of memantine as the first clinically successful NMDAR antagonist involved finding unique kinetics of drug action in the NMDAR-associated ion channel (Lipton, S. A. et al., N. Engl. J. Med. 330, 613-622 (1994); Lipton, S. A. Trends Neurosci 16, 527-532 (1993); Chen, H.-S. V. and Lipton, S. A., J. Physiol. (Lond.) 499, 27-46 (1997); Chen, H.-S. V. et al. J. Neurosci. 12, 4427-4436 (1992); Chen, H.-S. V. et al. Neuroscience 86, 1121-1132 (1998)). We found that the off-rate from the channel is a major determinant of clinical tolerability of open-channel blockers because an excessively slow off-rate (associated with a long dwell time) causes the drug to accumulate in the channels, interfere with normal neurotransmission and produce unacceptable adverse effects (as in the case of MK-801). In contrast, too rapid an off-rate yields a relatively ineffectual blockade, especially with membrane depolarization, which relieves the block of positively charged molecules (as seen with Mg²⁺). The apparent affinity of a positively charged channel blocker such as memantine at a particular membrane voltage is related to its off-rate divided by its on-rate. The on-rate is a property of the channel's open probability and the drug's diffusion rate and concentration. On the other hand, the off-rate is an intrinsic property of the drug-receptor complex, and is unrelated to drug concentration (Chen, H.-S. V. and Lipton, S. A., J. Physiol. (Lond.) 499, 27-46 (1997)). A relatively fast off-rate is a major contributor to a drug such as memantine's low affinity for the channel pore. Although many factors determine a drug's clinical efficacy and tolerability, it seems that the off-rate of open-channel blockers such as memantine is a major factor.

Memantine therefore represents a class of relatively low-affinity, open-channel blockers—that is, drugs that only enter the channel when it is opened by agonist. In the case of memantine, at concentrations administered to patients, the drug enters the channel preferentially when it is pathologically activated for long periods of time; for example, under conditions of excessive glutamate exposure. The relatively fast off-rate prevents the drug from accumulating in the ion channels and interfering with subsequent normal synaptic transmission. As a result memantine has favorable kinetics in the channel to provide neuroprotection while displaying minimal adverse effects (Lipton, S. A. et al., N. Engl. J. Med. 330, 613-622 (1994); Lipton, S. A., Trends Neurosci 16, 527-532 (1993); Chen, H.-S. V. and Lipton, S. A., J. Physiol. (Lond.) 499, 27-46 (1997)). Reported side effects include occasional restlessness (akathesia) or, in rare cases, slight dizziness at higher dosages than commonly used clinically.

ii. Kinetics of Memantine in NMDAR Channels

$$Channel + MEM ↔ {{Channel}^{{k}_{on}}}_{{k}_{off}} MEM ( Blocked channel ) \qquad ( 1 )$$

This simple bimolecular scheme predicts that the macroscopic blocking and unblocking actions of memantine (MEM) proceeds with exponential relaxation. The macroscopic pseudo-first order rate constant of blocking (kₒₙ) depends linearly on memantine concentration (as well as a constant, A), and the macroscopic unblocking rate (k_{off}) is independent of memantine concentration ([MEM]).

kₒₙ=A·[MEM] (2)

k_{off}: [MEM] independent (3)

These predictions were borne out experimentally (Chen, H.-S. V. et al. J. Physiol. (Lond.) 499, 27-46 (1997)). Both the macroscopic blocking and unblocking processes could be well fitted by a single exponential function. The macroscopic on-rate constant is related to the reciprocal of the measured time constant, which for onset (τₒₙ) is the sum of the pseudo-first order blocking rate constant (kₒₙ) and unblocking constant (k_{off}). The unblocking rate constant (k_{off}) is the reciprocal of the measured macroscopic unblocking time constant (k_{off}). These transformations lead to Equations (4) and (5):

kₒₙ=1/τₒₙ−1/τ_{off} (4)

k_{off}=1/τ_{off} (5)

The kon calculated from Equation 4 increased linearly with memantine concentration with a slope factor of 0.4±0.03 106 M−1 s−1 (mean±s.d.), while the k_{off} from Equation 5 remained relatively constant with a Y-axis intercept of 0.44±0.1 s−1 (Chen, H.-S. V. and Lipton, S. A., J. Physiol. (Lond.) 499, 27-46 (1997)). Here a rapid method was used to validate this result by estimating the apparent dissociation constant (Ki) at equilibrium for memantine action from the following equation:

Ki=k_{off}/(kₒₙ/[MEM]) (6)

Here it was found empirically that memantine was a relatively low-affinity (apparent affinity of ˜1 μM) open-channel blocker of the NMDAR-coupled ion channel, and a major component of the affinity was determined by k_{off} at clinically relevant concentrations in the low micromolar range.

6. History of Memantine as an NMDAR Antagonist

Memantine was first synthesized by Eli Lilly & Co. and patented in 1968, as documented in the Merck Index. Memantine is a derivative of amantadine, an anti-influenza agent and, like amantadine, memantine has a three-ring (adamantane) structure with a bridgehead amine (—NH₂) that is protonated under physiological conditions to carry a positive charge (—NH₃⁺). It was reported that memantine binds at or near the Mg²⁺ site in the NMDAR-associated channel (see FIG. 14) (Lipton, S. A., Trends Neurosci 16, 527-532 (1993); Chen, H.-S. V. and Lipton, S. A., J. Physiol. (Lond.) 499, 27-46 (1997); Chen, H.-S. V. et al., J. Neurosci. 12, 4427-4436 (1992); Chen, H.-S. V., et al. Neuroscience 86, 1121-1132 (1998); Chen, H.-S. V. and Lipton, S. A., J. Pharmacol. Exp. Ther. 314, 961-971 (2005)). Unlike amantadine, memantine has two methyl (—CH₃) side groups that prolong its dwell time in the channel, and which slow its off-rate and increase its affinity for the channel compared with amantadine. The reported efficacy of amantadine and memantine in Parkinson's disease, which was discovered by serendipity in a patient taking amantadine for influenza, led scientists to believe that these compounds were dopaminergic or possibly anticholinergic drugs. It was not until the late 1980s and early 1990s that memantine was found to be neither dopaminergic nor anticholinergic at its clinically useful dosage, but, rather, an NMDAR antagonist. Work at the small German pharmaceutical company Merz first indicated that the drug was a quite ‘potent’ NMDAR inhibitor (Bormann, J. Eur. J. Pharmacol. 166, 591-592 (1989)), which in fact it is not. Instead it was found that under physiological conditions the drug is of such weak potency at the NMDAR (affinity in the micromolar range rather than nanomolar or higher) that Big Pharma initially thought that it was a poor candidate as a neuroprotective drug. However, one should not confuse affinity with selectivity; as long as a drug acts selectively and specifically on the target of interest and the effective concentration can be achieved, high affinity per se is not the key issue. In fact, it was showed why memantine could be clinically tolerated as an NMDAR antagonist; namely, it was an uncompetitive, open-channel blocker with a relatively rapid off-rate from the channel. This constellation of properties allowed memantine to limit pathological activity of the NMDAR while relatively sparing normal synaptic activity (Chen, H.-S. V. et al., J. Neurosci. 12, 4427-4436 (1992); Chen, H.-S. V. et al., Neuroscience 86, 1121-1132 (1998); Chen, H.-S. V. and Lipton, S. A., J. Pharmacol. Exp. Ther. 314, 961-971 (2005)). These findings led to a number of US and worldwide patents on the use of memantine for NMDAR-mediated disorders and spurred on several successful clinical trials with the drug, as discussed below.

To help visualize the blockade of NMDA-induced ionic currents by memantine, a sample experiment is illustrated in FIG. 15 in which the membrane voltage of a neuron was held at approximately the resting potential. The low micromolar concentration of memantine used in this experiment is similar to the level that can be achieved in the human brain when the drug is used clinically. At such concentrations, memantine greatly reduces pathologically high levels of NMDA-induced current in ˜1 second. Once the memantine application stops, the NMDA response returns to previous levels over a period of about 5 seconds (FIG. 15, heavy trace). This indicates that memantine is an effective, but temporary, NMDAR blocker. In contrast, MK-801 block persists for a very long period after washout (FIG. 15, light trace, upper right), producing accumulation of the drug in NMDAR-operated channels and blockade of subsequent normal activity. The relatively rapid off-rate of the effect of memantine is crucially important for its clinical tolerability, as discussed below.

Perhaps the most intriguing property of memantine is shown in FIG. 16 (Lipton, S. A., Trends Neurosci 16, 527-532 (1993); Chen, H.-S. V. et al., J. Neurosci. 12, 4427-4436 (1992)). In this experiment, the concentration of memantine was held constant (at a clinically achievable level of 1 μM) while the concentration of NMDA was increased over a wide range. It was found that the degree to which this fixed concentration of memantine blocked NMDAR activity actually increased as we increased the concentration of NMDA to pathological levels. In fact, memantine was relatively ineffectual at blocking the low levels of receptor activity associated with normal neurological function but became exceptionally effective at higher concentrations. This behavior represents classical ‘uncompetitive’ antagonism.

Further studies indicate that memantine exerts its effect on NMDAR activity by binding at or near the Mg²⁺ site within the ion channel (Lipton, S. A., Trends Neurosci 16, 527-532 (1993); Chen, H.-S. V. and Lipton, S. A., J. Physiol. (Lond.) 499, 27-46 (1997); Chen, H.-S. V. et al., J. Neurosci. 12, 4427-4436 (1992); Chen, H.-S. V. et al., Neuroscience 86, 1121-1132 (1998); Chen, H.-S. V. and Lipton, S. A., J. Pharmacol. Exp. Ther. 314, 961-971 (2005)). Recently, it was showed that the Mg²⁺ binding site on the NR1 rather than the NR2 subunit is most intimately involved in memantine binding, representing the so-called ‘intracellular’ Mg²⁺ site (Chen, H.-S. V. and Lipton, S. A., J. Pharmacol. Exp. Ther. 314, 961-971 (2005)). These new data readily explain the prior observation that memantine blocks all types of NMDAR-operated channels that comprise NR1 and various NR2 subunits (Bresink, I. et al., Br. J. Pharmacol 119, 195-204 (1996).; Chen, H.-S. V. and Lipton, S. A. in Post Genomic Drug Discovery Research (ed. Huang, Z.) (John Wiley & Sons, Hoboken, N.J. (in the press)). Previously, this observation was puzzling because particular NR2 subunits (that is, NR2c and NR2D) are less sensitive to inhibition by Mg²⁺, so it was thought that NMDARs containing these subunits would be less affected by memantine. However, the fact that memantine blocks at the NR1 rather than the NR2 subunit affords an explanation for the effectiveness of memantine at all NMDARs.

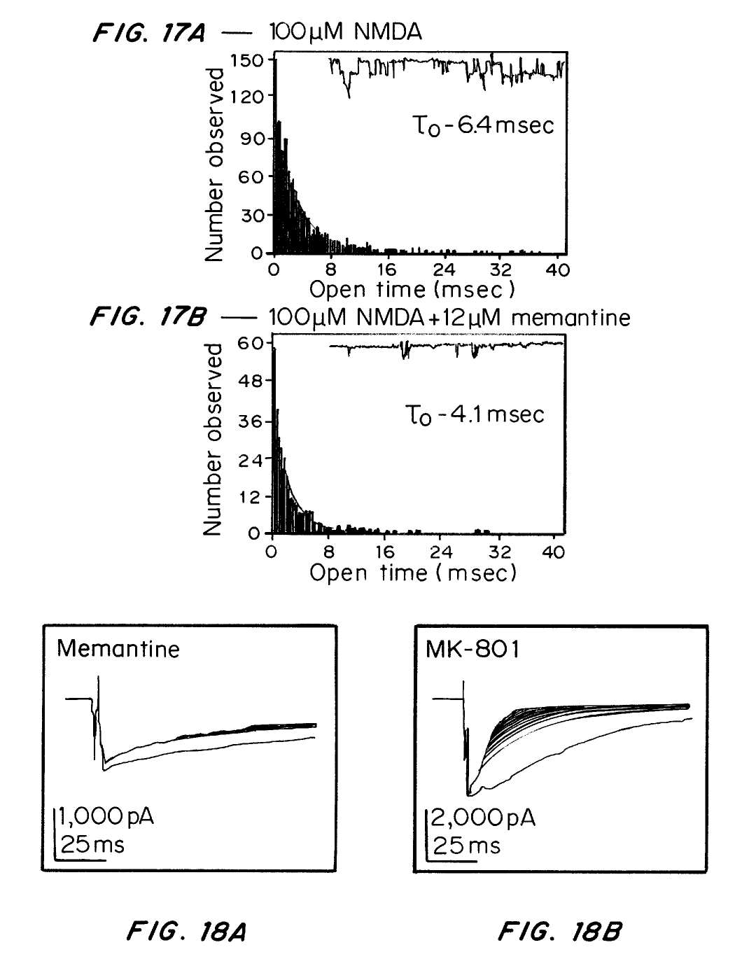

The information on memantine blocking at the so-called internal Mg²⁺ site on the NR1 subunit, coupled with the pharmacological/kinetic data presented earlier, indicates that memantine preferentially blocks NMDAR activity if the ion channel is excessively open. FIG. 17 highlights this effect of memantine at the single-channel level, showing in an outside-out patch from a neuron that channel activity is damped down after memantine addition (compare right side (b) FIG. 17 with left (a)). Statistical evaluation of long epochs of such traces showed that memantine was blocking the open channels (Chen, H.-S. V. and Lipton, S. A., J. Physiol. (Lond.) 499, 27-46 (1997); Chen, H.-S. V. et al., J. Neurosci. 12, 4427-4436 (1992)).

However, during normal synaptic activity, NMDAR-associated channels are open on average for only several milliseconds, and memantine is unable to accumulate in the channels so that synaptic activity continues essentially unabated. This concept is illustrated in FIG. 18, which shows that the initial AMPA receptor-mediated portion of excitatory synaptic current is unaffected by the NMDAR antagonists memantine (left-hand panel) or MK-801 (right-hand panel). However, MK-801 accumulates in a use-dependent manner in the channels to block the late NMDAR-mediated component of synaptic currents, whereas memantine blocks very little (10-15%) of this component.

In contrast, during prolonged activation of NMDARs, as occurs under excitotoxic conditions, memantine becomes a very effective blocker (FIG. 15). In essence, memantine only acts under pathological conditions without much affecting normal function, and so relatively spares synaptic transmission. It was also shown that this relative sparing of normal neurotransmission results in the preservation of NMDAR-mediated long-term potentiation as well as physiological function when monitored with behavioural tests such as the Morris water maze (Chen, H.-S. V. et al., Neuroscience 86, 1121-1132 (1998)). Additionally, preserved synaptic activity is associated with the absence of structural defects that other NMDAR antagonists cause, such as vacuolarization of adult cingulate neurons and apoptosis of perinatal neurons (Chen, H.-S. V. et al., Neuroscience 86, 1121-1132 (1998)). The kinetics of memantine action in the NMDAR-associated ion channel explain this favourable clinical safety profile because normal synaptic activity is preserved.

Additional effects of memantine: voltage dependence, partial trapping and other mechanisms. It was reported that memantine block is voltage-dependent like magnesium ions (Chen, H.-S. V. and Lipton, S. A., J. Physiol. (Lond.) 499, 27-46 (1997); Chen, H.-S. V. et al., J. Neurosci. 12, 4427-4436 (1992)) (recall that memantine is positively charged at physiological pH). During depolarization caused by excitatory neurotransmission (excitatory postsynaptic potentials), memantine block would therefore be partially relieved. Nonetheless, it was shown that even when voltage-clamped to avoid such depolarization, memantine does not block synaptic activity to any substantial degree (Chen, H.-S. V. et al., Neuroscience 86, 1121-1132 (1998)). The voltage-dependence of memantine is therefore not the major factor for its lack of effect on synaptic activity as some have claimed. Rather, the relatively fast off-rate from the ion channel seems to be a crucial feature, as explained above. Additionally, Chen, H.-S. V. and Lipton, S. A., J. Physiol. (Lond.) 499, 27-46 (1997), and Blanpied, T. A. J. Neurophysiol. 77, 309-323 (1997), demonstrated that if agonist is washed out quickly, then memantine can be trapped in the NMDAR-associated ion channel (that is, the channel shuts behind memantine and traps it in the closed channel). However, since memantine does not block a substantial proportion of channels during normal synaptic activity, trapping in the channel cannot be a major factor under physiological conditions. Moreover, because rapid washout of agonist is required to observe trapping, this mechanism is not relevant under pathophysiological conditions when prolonged glutamate exposure activates, for example, extrasynaptic receptors. Trapping of a related but less potent NMDAR antagonist, amantadine, has recently been reported to account, at least in part, for the drug's action (Blanpied, T. et al., J. Neurosci. 25, 3312-3322 (2005)). However, the presence of two low-affinity sites for amantadine in the NMDAR associated channel (Chen, H.-S. V. and Lipton, S. A., J. Pharmacol. Exp. Ther. 314, 961-971 (2005); Chen, H.-S. V. and Lipton, S. A. in Post Genomic Drug Discovery Research (ed. Huang, Z.) (John Wiley & Sons, Hoboken, N.J. (in the press)) represents an alternative explanation for these findings rather than physiological trapping. Additionally, closed-channel block (entering the channel before it has opened) has been proposed as a mechanism of action of memantine (Sobolevsky, A. et al., J. Physiol. (Lond.) 512, 47-60 (1998)), but this occurs only at very high concentrations of memantine, so this action is not pathophysiologically relevant under clinical conditions (Chen, H.-S. V. and Lipton, S. A., J. Physiol. (Lond.) 499, 27-46 (1997). Finally, memantine can also block 5-hydroxy tryptamine (5-HT)3 receptor-channels and α7 nicotinic receptor channels but, when measured at equilibrium, at apparently higher concentrations than those that block NMDAR-channels (Chen, H.-S. V. and Lipton, S. A., J. Physiol. (Lond.) 499, 27-46 (1997); Chen, H.-S. V. et al., J. Neurosci. 12, 4427-4436 (1992); Chen, H.-S. V. et al., Neuroscience 86, 1121-1132 (1998); Chen, H.-S. V. and Lipton, S. A. in Post Genomic Drug Discovery Research (ed. Huang, Z.) (John Wiley & Sons, Hoboken, N.J. (in the press); Rammes, G. et al. Neurosci. Lett. 306, 81-84 (2001); Reiser, G. Brain Res. 443, 338-344 (1988)). An effect of memantine on 5-HT3 receptors might enhance cognitive performance, whereas blockade of presynaptic nicotinic channels might decrease glutamate release.

Neuroprotective efficacy of memantine. The neuroprotective properties of memantine have been studied in a large number of in vitro and in vivo animal models by several laboratories (Parsons, C. G. et al., Neuropharmacology 38, 735-767 (1999). Neurons protected in this manner both in culture and in vivo include cerebrocortical neurons, cerebellar neurons and retinal neurons (Lipton, S. A., Trends Neurosci 16, 527-532 (1993); Chen, H.-S. V. and Lipton, S. A., J. Physiol. (Lond.) 499, 27-46 (1997); Chen, H.-S. V. et al. J. Neurosci. 12, 4427-4436 (1992); Chen, H.-S. V. et al., Neuroscience 86, 1121-1132 (1998); Vorwerk, C. K. et al. Invest. Ophthalmol. Vis. Sci. 37, 1618-1624 (1996); Lipton, S. A., Neurology 42, 1403-1405 (1992); Pellegrini, J. et al. Ann. Neurol. 33, 403-407 (1993); Sucher, N. et al. Vision Res. 37, 3483-3493 (1997); Osborne, N. Vis. Neurosci. 16, 45-52 (1999)). Additionally, in a rat model of stroke, memantine, given up to 2 hours after the ischaemic event, reduces brain damage by approximately 50% (Chen, H.-S. V. et al., J. Neurosci. 12, 4427-4436 (1992); Chen, H.-S. V. et al. Neuroscience 86, 1121-1132 (1998)). An increasing number of clinical trials have proven memantine to be an effective treatment for neurodegenerative conditions. Interestingly, the fact that the beneficial effect of memantine was observed during the washout period for a month or more in some human clinical trials is consistent with the drug manifesting a neuroprotective profile (Navia, B. A. Neurology 64 (Suppl. 1), A247-A238 (2005)).

A series of human clinical trials have been completed or nearly completed that test the efficacy of memantine in Alzheimer's disease, vascular dementia, HIV-associated dementia, diabetic neuropathic pain, depression and glaucoma. One high-profile publication reported the results of a US Phase III (final) clinical study showing that memantine (20 mg per day) is efficacious in slowing moderate-to-severe Alzheimer's disease (Reisberg, B. et al., N. Engl. J. Med. 348, 1333-1341 (2003)). Another study reported that, in combination with donepezil (Aricept; Eisai/Pfizer), memantine treatment improved cognitive function to some degree in moderate-to-severe Alzheimer's patients (Tariot, P. N. et al., JAMA 291, 317-324 (2004)). These results convinced the European Union to approve memantine for Alzheimer's disease 3.5 years ago, and the US FDA in late 2003. Concerning other forms of dementia, a European multicentre, randomized controlled trial reported that memantine was beneficial in severely demented patients, probably representing both Alzheimer's disease and vascular dementia (Winblad, B. et al., Int. J. Geriat. Psych. 14, 135-146 (1999)). Still another recent publication described significant benefit from in mild-to-moderate vascular dementia (Orgogozo, J. M. et al., Stroke 33, 1834-1839 (2002)). Most trials have reported minimal adverse effects of memantine. Rarely encountered were dizziness and restlessness/agitation (but usually at higher doses of 40-60 mg per day).

Importantly, the counterintuitive aspect of memantine action that was discovered, showing that more glutamate receptor activity is blocked better than less activity at a fixed dose of memantine, has clinical implications. Memantine would be expected to work better for severe conditions. A case in point is neuropathic pain, thought to be mediated at least in part by excessive NMDAR activity. A Phase IIB clinical trial indicated that more severe (nocturnal) pain was abated more than mild (daytime) pain, and, in fact, milder pain conditions did not statistically benefit from memantine in a Phase III clinical trial. Similarly, for mild Alzheimer's disease memantine has been found to be useful in some studies but not in others, leading to the recent FDA decision to put approval of memantine on hold for mild dementia in contrast with its approval for moderate-to-severe disease.

Paradoxically, the work would also predict that a higher concentration of memantine would be needed to combat pain syndromes or mild dementia than to prevent the more severe synaptic damage and neuronal cell death seen in moderate-to-severe dementia, because greater NMDAR activity is associated with severe synaptic damage and cell death. Recall that the on-rate of channel block by memantine can be increased by increasing the drug's concentration, resulting in a greater proportion of channels blocked. Again, clinical trials have indicated that this scenario is indeed correct because 40 mg per day of memantine have been needed in successful studies of nocturnal pain but only 20 mg per day in studies of moderate-to-severe dementia. Following this line of reasoning, one would predict that higher concentrations of memantine (≧40 mg per day) would be necessary for optimal treatment of mild dementia. On the other hand, as alluded to above, higher doses increase the risk of side effects.

7. NitroMemantines

As promising as clinical trials are with memantine, additional modulatory sites (‘volume controls’) can be used to block excitotoxicity even more effectively. S-Nitrosylation by an NO or ‘nitro’ group (for example, nitroglycerin) of crucial cysteine residue(s) on the NMDAR downregulates excessive receptor activity (see FIG. 13) (Lipton, S. A., Trends Neurosci 16, 527-532 (1993); Dreyer, E. B., NeuroReport 6, 942-944 (1995); Lipton, S. A. et al., Trends Neurosci. 25, 474-480 (2002); Lipton, S. A. & Wang, Y. F. in Pharmacology of Cerebral Ischemia (ed. Krieglstein, J.) 183-191 (Medpharm Scientific, Stuttgart, 1996); Zurakowski, D. et al. Vision Res. 38, 1489-1494 (1998); Lipton, S. A., et al. in Prog Brain Res. (eds Mize, V., Dawson, T. M., Dawson, M. & Friedlander, M.) 73-82 (Elsevier, Amsterdam, 1998); Choi, Y.-B. et al. Nature Neurosci. 3, 15-21 (2000)). Unfortunately, nitroglycerin itself is not very attractive as a neuroprotective agent because it could cause dangerously large drops in blood pressure. Consequently, a nitro group was attached to memantine in order to target NMDARs of overly active neurons. These drugs, called NitroMemantines, represent second-generation memantine derivatives that are designed to have enhanced neuroprotective efficacy without sacrificing safety. Two sites of modulation are analogous to having two volume controls on your television set for fine tuning. Preliminary studies have shown that NitroMemantines are substantially more effective than memantine as neuroprotectants, both in vitro and in vivo in animal models, and do not display any ill effect on blood pressure. The surprisingly yet clinically tolerated action of uncompetitive, low-affinity/fast off-rate drugs in animal models and clinical studies of both acute and chronic neurological disorders indicates that memantine and newer derivatives could become very important weapons in the fight against neuronal damage (Le, D. A. et al. Drugs Aging 18, 717-724 (2001); Lipton, S. A., Nature 428, 473 (2004); Lipton, S. A. et al., Cell Death Differ. 11, 18-20 (2004)). Additionally, evidence demonstrates that NMDARs are also present on the processes of oligodendrocytes, constituting the myelin sheath of CNS axons, and contribute to injury, cell death and demyelination from exposure to glutamate. For this reason, NMDAR antagonists such as memantine and the newer NitroMemantines can be used in white-matter (myelin covering axonal cylinders) as well as grey-matter (neuronal) diseases, including multiple sclerosis and periventricular leukomalacia. Moreover, by virtue of their relatively gentle binding, drugs of this genre work best under pathological conditions, while exerting minimal effects on normal brain activity. As described herein, this concept can be extended to other neuroprotective targets and even to other pharmaceutical agents in general.

### D. Example 4

### Platform to Discover Pathologically-Activated Therapeutics (PAT Drugs)

Many if not all drugs, particularly when developed against targets in the brain, manifest unacceptable clinical side effects. Described herein is a platform to discover pharmacological therapeutic agents that are pathologically activated, thereby avoiding side effects, using high-throughput screening (HTS) assays of chemical libraries combined in an iterative fashion with structural biology (crystallographic structural analysis with in silico screens). The ‘initial hits’ in this approach to drug discovery finds cryptic sites on drug targets that are exposed only upon excessive activation by agonist. Developing antagonists to these ‘cryptic’ sites insures that only excessive (pathological) activity of the drug target is curtailed by the drug thus developed, while sparing normal activity of the target.

The described methods alter the way that biopharmaceuticals are discovered. The disclosed methods are a whole new way to look at how to discover new drug therapies.

Many drugs that have been developed to treat degenerative diseases fail to gain approval for clinical use because they are not well tolerated in humans. Described herein is a series of strategies for the development of protective therapeutics that are both effective and well tolerated. These strategies are based on the principle that drugs should be activated by the pathological state that they are intended to inhibit. Described herein is a systematic way to screen for such clinically-tolerated yet efficacious drugs in order to revolutionize and improve drug discovery.

1. Methods

The disclosed methods screen for uncompetitive drugs against a number of targets. For example, one technique involves using fluo-4 NW, a recently-developed and improved intracellular Ca²⁺ indicator dye not requiring washout from the solution and hence suitable for high-throughput screening (HTS) of chemical libraries. Primary neurons or cell lines are used that contain (or are genetically engineered to contain) a target molecule on their surface, such as the AMPA/kainate-type glutamate receptors with unedited GluR2 subunits or other molecules that result in intracellular Ca²⁺ signaling, such as G-protein-coupled receptors. Glutamate receptors containing unedited GluR2 subunits are thought to be important in mediating neuronal damage in global ischemia and in sporadic cases of ALS (amyotrophic lateral sclerosis), and hence represent an important drug target without clinical drugs to date. Screening (by quantifying the Ca²⁺ response) with two concentrations of agonist for the target, a low concentration and a higher concentration, against chemicals in the library administered at a single micromolar concentration. For these AMPA receptors, kainate can be used as the agonist for HTS because the responses are less desensitizing than those to AMPA. The agonist results in an increase in intracellular Ca²⁺ that can be monitored by HTS using the Ca²⁺ indicator dye fluo-4 NW.

The screening can be applied to any compounds. For example, known drugs, obtained from a library of FDA-approved drugs, small chemical libraries, such as NIH Clinical Collection (450 compounds) or Sigma LOPAC library (1,280 compounds), larger chemical libraries, such as the Chembridge DIVERSet Collection (˜50,000 compounds), and the Burnham Collection with 100,000 to 150,000 compounds. A positive ‘hit’ in an uncompetitive screen such as those described here quite paradoxically will find that responses to a high concentration of agonist are inhibited to a greater degree percentage-wise than responses to a lower concentration of agonist at a fixed dose of the antagonist drug being screened for. As discussed elsewhere herein, uncompetitive antagonists that are clinically tolerated are generally of relatively low (micromolar) affinity because they have rapid off-rates (which is desired so that the antagonists drug does not accumulate and block normal responses after excessive activity has abated). As a validation of this approach, the action of memantine, known to work as an uncompetitive antagonist at the NMDA receptor as the target, has already been successfully achieved in a preliminary screen.

2. Results

Using cerebrocortical neurons and calcium-sensitive dyes, as described above, conditions have been optimized for screening for AMPA/Kainate-receptor antagonists. For this purpose, the following experimental parameters were varied: cell density, composition of culture medium, length of cultures, type of calcium-sensitive dye, and condition (temperature and length) of dye loading. The following protocol was established. Neurons are plated at 15K/well in 384-well plates and cultured in D10C medium for 13 days before they are used for HTS.

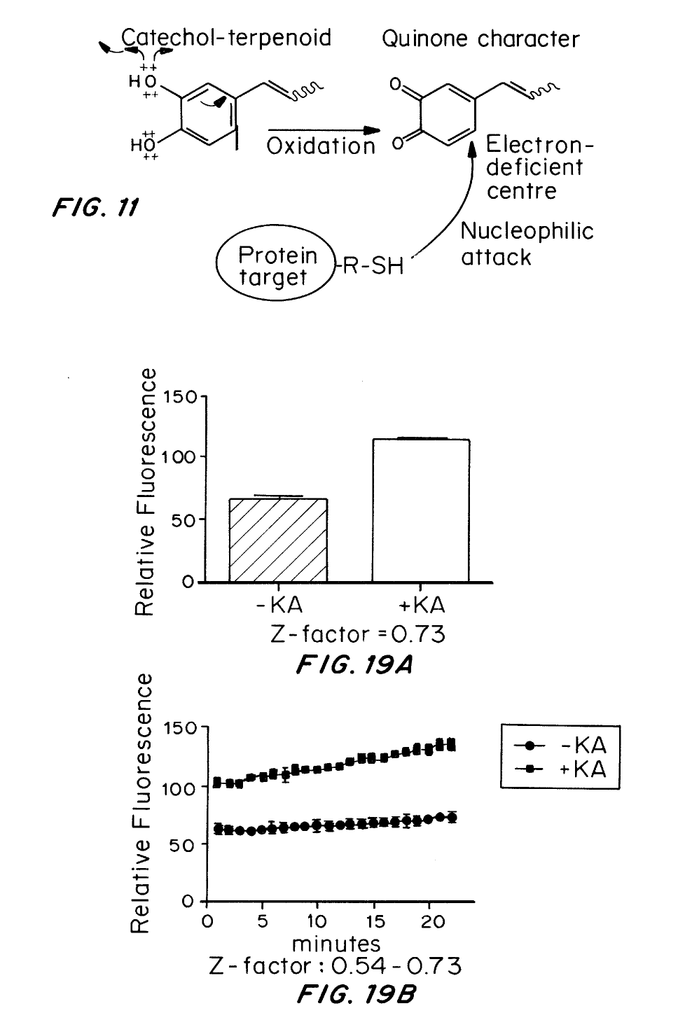

Next a primary screen is performed of the LOPAC 1280 library for potential inhibitors of KA using the fluorescent assay shown in FIG. 19. In three separate screens, 9, 23, and 14 compounds were identified that significantly blocked calcium entry induced by KA. Strikingly, there were two ‘hits’ representing compounds that were scored positively in all three screens, and they were NBQX and DNQX, established AMPA/KA-type glutamate receptor antagonists. In addition, three other compounds were positively scored, and one of them was CNQX, another established AMPA/KA-type glutamate receptor antagonist. These preliminary results indicate that the established conditions are appropriate for HTS. Sanford-Burnham Medical Research Institute's HTS Core Facility estimates that this screen is sufficiently robust to be able to screen approximately a million compounds in the Chemical Libraries in a two-week period. A secondary screen can then be used to determine if the “hits” represent an true uncompetitive antagonist, which at a fixed dose would block a higher concentration of agonist (e.g., 200 μM KA) to a greater degree than a lower concentration of agonist (e.g., 20 μM KA). Finally, as part of the screening process, if the test compound acts in such an uncompetitive manner, then a tertiary screen can be performed by, for example, whole-cell patch-clamp recording under steady-state application of the agonist to monitor the kinetics of action of the inhibitory compound to insure that it has a relatively fast off-rate, the other critical determinant in UFO action, as described above. As described below, an automated procedure for patch clamping is available that approaches high-throughput capacity using the AVIVA, Inc. automated system. Importantly, an unedited GluR2 cell line in HEK293 cells is currently made for HTS because screening with cell lines will produce less background in the assays, and therefore even more reliable screens.

3. Alternative Primary and Secondary Screening Methods

Off-target effects, particularly of low-affinity ‘hits,’ can be ruled out by counter-screening against other targets. For example, NMDA-type glutamate receptor-expressing cells lines can be used for this purpose if we are screening for AMPA-type glutamate receptor antagonists. Additionally, screens against other targets to insure selectivity can be used when optimizing hits, for example a commercially available binding assay can be used that will rule out binding to approximately 100 other channel and G-protein related targets.

Primary neurons can be used for screening. However, the use of primary neurons in a screen can exhibit background fluorescence and variability in the screening process due to heterogeneity of cells. In fact, some variability in the Z values have been observed while developing a screening assay using primary neurons. As an alternative, cell lines can be used. For example, several stable cell lines that express a variety of AMPA/KA-type glutamate receptor (GluR) subunits can be used, including, for example, various forms of GluR1 and GluR2. We initially used a homomeric receptor consisting of an ‘unedited’ form of GluR2 (designated GluR2Q) expressed in an HEK293 cell line for HTS because, as discussed elsewhere herein, unedited GluR2 is permeable to Ca²⁺ (therefore, allowing the use of Ca²⁺-sensitive dye for monitoring receptor function), and screening with these cell lines will produce less background fluorescence in the assays, therefore affording even more reliable screens. When we used a GluR2Q cell line with the improved Ca²⁺-sensitive dye, Calcium 5 FLIPR, improved and more reliable z values=0.8 were observed (see FIG. 28). A FlexStation 3 Plate reader from Molecular Devices was used to monitor the fluorescence intensity change (termed a Fluorescent Imaging Plate Reader (FLIPR) assay).

In another example of primary and secondary screening procedure, instead of using calcium-sensitive dyes, voltage changes induced by receptor activation using the membrane potential FLIPR kit assay can also be monitored by utilizing a dye sensitive to membrane potential (or voltage) changes (see FIG. 20). This approach allows screening of receptor-channels that are not as permeable to Ca²⁺ but do allow flux of other ions, resulting in a membrane potential change in the cells.

HEK293 cells stably expressing AMPA/KA glutamate receptors may succumb to glutamate-related toxicity (termed excitotoxicity) because of chronic stimulation with glutamate, which can arise, for example, from glutamine in the media. To avoid this, inducible forms of recombinant AMPA/KA receptors were also produced (whose subunits are designated as GluRs) using a doxycycline-inducible (tet-on) system. This can be an important advantage in that expression of the receptors can be turned “on” when desired, thereby avoiding glutamate-mediated damage to the cell line by keeping glutamate receptor expression switched “off” until a screen is ready to be run. An example of a membrane potential assay on such a cell line is shown in FIG. 20.

Below are examples of various AMPA/KA-type glutamate receptor cell lines available to be used for screening. The following GluR subunits stably expressed in HEK293 cells lines are available, which can used for optimizing the HTS procedures:

Calcium permeable HEK lines: GluR1, GluR2Q, GluR3, and GluR4 for AMPA receptors; GluR5Q and GluR6Q for Kainate receptors. Each of these lines represent homomeric channels (each receptor contains only one kind of subunit).

Calcium impermeable HEK lines: GluR1/GluR2 heteromeric AMPAR channels (this line contains a receptor composed of GluR1 and GluR2 subunits. The GluR2 subunit was edited (Q to R), and hence not very permeable to calcium ions, but mimics many native channels of this sort that are encountered in the brain. Note that the cell line expressing calcium impermeable receptor-channels should be screened with a membrane voltage sensitive dye (as shown in FIG. 20) because of the lack of Ca²⁺ influx via these channels. Note that several of these lines also express GFP, so it can readily be determined if the receptors are expressed in the cells, and GFP does not interfere with the fluorescence assays that were chosen for HTS.

4. Tertiary Screen Using AVIVA, Inc. Technology for HTS Patch Clamping

Recently, another type of screen was developed that can look for UFO drugs, i.e. uncompetitive inhibition with a fast off-rate, in a single step. Since this screen is “slower,” more expensive, and labor intensive than the initial fluorescent screen with the Ca²⁺-sensitive fluo-4 NW dye (described above), this automated patch-clamp procedure can most effectively be used as a tertiary screen for initial ‘hits’ obtained from large chemical libraries or from ‘hits’ after optimization because the patch-clamp data can give the kinetic information of antagonist action needed to determine if the uncompetitive antagonist of interest identified by the initial fluorescent screen has a sufficiently fast off-rate to predict clinical tolerability. In this type of screen, which works for receptors coupled to ion channels, a new automated patch-clamp system is used that in a multiwell plate can monitor the effects of two concentrations of antagonist, looking for uncompetitive behavior, while assaying the kinetics of the on-rate and off-rate of the antagonist during whole-cell recording. In this automated patch-clamp screen, all the information needed can be obtained to determine if a drug in a chemical library exhibits UFO behavior in a single step and with much faster throughput than by conventional whole-cell recording with a patch electrode (Lipton, S. A., Nature Rev. Drug Discov. 5, 160-170 (2006); Chen, H.-S. V. and Lipton, S. A., J. Physiol. 499, 27-46 (1997); Chen, H. S. V. et al. J. Neurosci. 12, 4427-446 (1992)). In one day, the effect of 50-80 of the initial ‘hits’ can be monitored using this method, whereas using conventional patch clamping only 1-2 drugs could be monitored in a single day. The on and off rates of the drug during application and, after reaching a steady state effect, during washout, as well as the effect of various concentrations of a presumptive antagonist was determined using AVIVA, Inc. automated patch clamp for HTS. Multiple drug additions were monitored by HT (hight-throughput)-automated patch-clamp recording and showed resolution of kinetics (on and off rates of drug action). The IC₅₀ (concentration of antagonist that inhibits 50% of the response) calculated for antagonists added during automated patch-clamp recording with the AVIVA, Inc. instrument and software was calculated to be 0.29±0.03 μM (using percent block versus the concentration of antagonist for a single concentration of agonist).

It should be noted that heretofore developed methods for HTS using only ‘high-affinity screens’ have not been capable of discovering uncompetitive drugs like the assay described herein, so pharmaceutical companies have not performed this type of screen to date for any target. Once a “hit” or lead compound is identified, it can be optimized by chemists as discussed below. In an iterative fashion, optimized antagonist drugs can be re-run through the screen in an effort to increase the degree of potency a fixed dose of antagonist for blocking higher concentrations of agonist to a greater degree than lower concentrations of agonist. Crystallographic/structural data can also be used to optimize antagonist design in an iterative fashion as discussed below, following the next section which describes the chemical libraries to be screened.

5. Iterative Process of Drug Optimization with Crystallography Structures

Also described herein are methods for taking an initial hit from the UFO screening assays as described elsewhere herein and using the atomic structure of the target for drug optimization. As an example, the channel structure for GluR2 homomeric AMPA receptor-operated channels was recently solved (Sobolevsky, A. I. et al., Nature 462, 745-756 (2009)). The channel structure can be used to optimize the hits from the initial screens using in silico computational modeling, which can fit drug structures to the target channel crystal structure. Using these in silico programs, medicinal chemistry efforts can be guided at enhancing compound potency for the drug antagonists to be developed. In this manner, it will be possible to iteratively use the crystal structure to improve the structure of a ‘hit’ in order to increase its potency, which will allow medicinal chemists to rationally design improved UFO drugs. In a similar fashion, the coordinates of the structures of other target molecules can be used to refine drug hits from additional high-throughput screens for UFO drugs against these other targets. This iterative nature of the drug discovery process for UFO drugs—obtaining a hit from an initial HTS screen, refining it with in silico programs using the crystal structure, and then optimizing the drug—provides a rational way forward in optimizing such UFO drugs.

6. Medicinal Chemistry

The disclosed methods can make use of small molecule reagents, design and execution of structure-activity relationship (SAR) studies, and hit-to-lead optimization. These reagents are useful for mechanistic studies as well as advanced medications development studies. Synthetic compounds and analysis can be used for the development of small molecules as mechanistic biochemical probes and as drug candidates. Once identified through primary screening efforts, ‘hits’ can be re-synthesized and analyzed to validate potency of the pure compounds. The disclosed methods can also make use of hit scaffolds and smart libraries of analogs with the goal of improving potency and other pharmacological properties. Complete characterization of synthesized compounds can include, for example, TLC, HPLC, GC, UV-vis, NMR, and MS. Analysis related to chiral purity can also be performed. Small molecules can be developed into affinity reagent probes for biochemical target identification. An iterative process known as Dynamic Medicinal Chemistry can be used to put analogs through rounds of biological testing. The results can be analyzed to guide further chemical optimization using in silico and further rounds of UFO drug screening procedures. Compounds can be evaluated with regard to aqueous solubility, chemical stability, lipophilicity, and permeability. Lead compounds from in vitro testing can be optimized for in vivo administration in terms of overall bioavailability and metabolic stability, and for distribution and toxicity in small animals. Additional pharmacological data can be used to prioritize hits will include receptor binding and functional studies, protein binding, and in vivo pharmacokinetics.

The following is an example of this approach to drug screening:

1. Screening for AMPA-type glutamate receptors using Ca²⁺ indicator dye and high-throughput patch-clamp recordings in multiwell plates. Comparison with memantine, known to work as an uncompetitive antagonist at the NMDA receptor.

2. Screening for uncompetitive AMPA-type glutamate receptor antagonists. As an alternative, additional screens for G-protein-mediated receptors that signal via intracellular Ca²⁺ can be done, allowing to use the Ca²⁺ indicator dye in the HTS procedure to obtain antagonist drugs against additional targets. The UFO nature of the drug hits can be assessed in these screens.

3. Screening of AMPA-type glutamate receptor uncompetitive antagonists and optimization of drugs from in silico model fitting to crystal structures with reiterative screening.

Alternatively, screening for G-protein can be done by mediated receptor uncompetitive antagonists.

Other targets can be considered for the development of the screens described herein. The UFO nature of the drug hits can be assessed in these screens.

4. Lead compounds for AMPA-type glutamate receptor uncompetitive antagonists can be identified after optimization.

Drug optimization with reiterative screening can continue for G-protein, coupled receptors.

As alternative paths, screening against other targets can be performed. The UFO nature of the drug hits will be assessed in these screens.

5. Lead compounds for G-protein, coupled receptor antagonists can be identified after optimization.

### E. Example of Scalable Assay Approach for Neurobiological Function

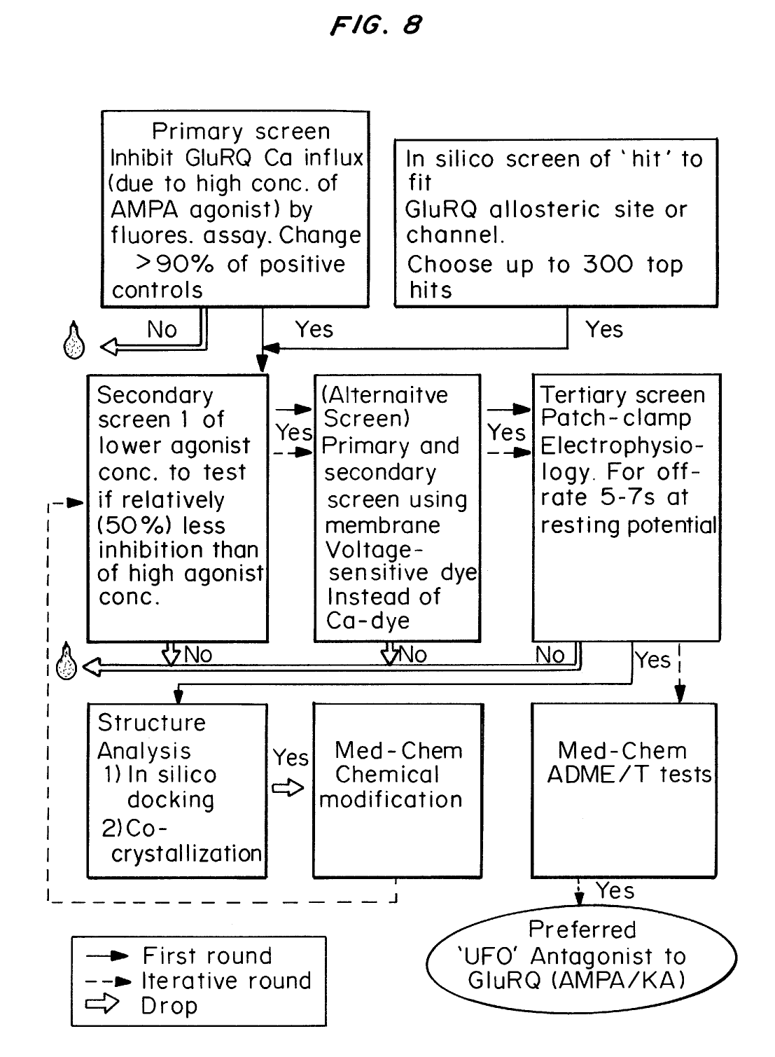

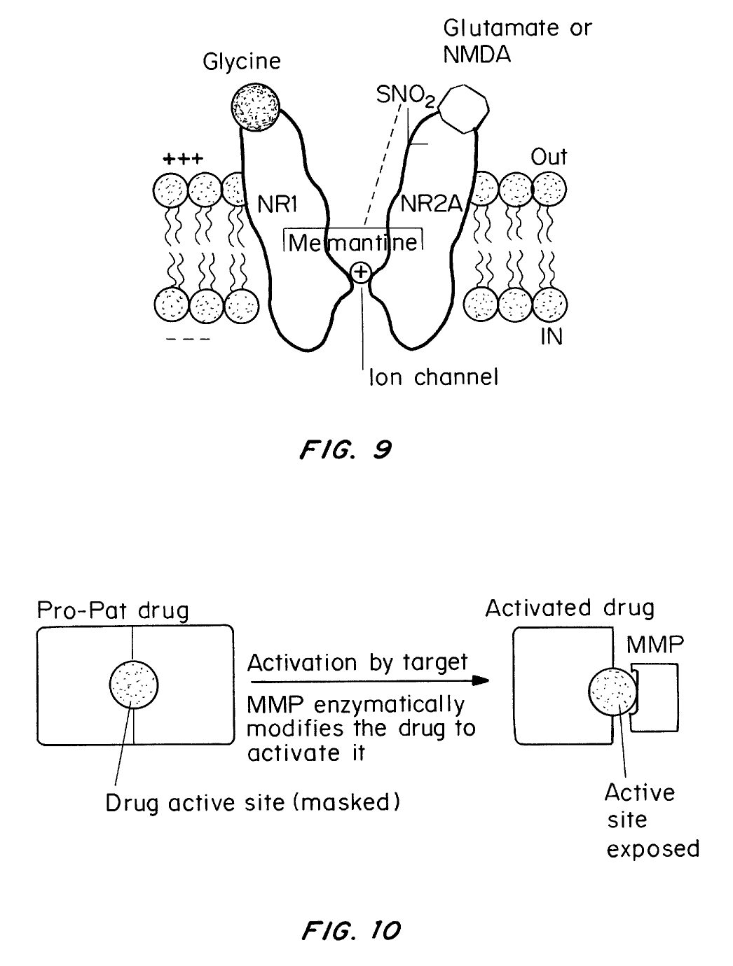

As an example of some methods described herein, FIG. 8 shows a workflow diagram, illustrating an example of an iterative process for screening and working up ‘hits’ for uncompetitive/fast off-rate (UFO)-type drugs with med-chem approaches. FIG. 8 outlines the primary screen, with a fixed dose of putative antagonist in a chemical library, initially tested against a high concentration of agonist. A secondary screen can then be performed on the initial ‘hits’ against a lower concentration of agonist to test if the relative degree of blockade is greater against the higher concentration of agonist, as found for an uncompetitive inhibitor. An alternative screen using voltage-sensitive fluorescent dyes can be used instead of (or in addition to) Ca²⁺-sensitive fluorescent dyes. A tertiary screen can then be performed involving, for example, moderate-throughput patch-clamp recording to resolve the kinetics of action of a ‘hit’ compound in order to determine if it has a relatively fast off-rate from the receptor target. If a ‘hit’ is thus determined from these screens to be an uncompetitive/fast off-rate (UFO) antagonist against the AMPA/kainate-type glutamate receptor, then structural analysis (with, for example, in silico docking and possible co-crystallization of drug and target) can be performed in an iterative fashion with the assays to help guide med-chem efforts at compound optimization. Eventually, lead compounds can be taken through ADME/T (absorption, distribution, metabolism, excretion, and toxicology) testing in rodents. Different assays, a different order of assays, and/or different iterations can be used in screening processes of this type. Any target molecule or enzyme and any lead or “hit” compounds can be used in similar staged and/or iterative processes.

### REFERENCES

Ankarcrona, M. et al. Glutamate-induced neuronal death: A succession of necrosis or apoptosis depending on mitochondrial function. Neuron 15, 961-973 (1995).
Asahi, M. et al. Effects of matrix metalloproteinase-9 gene knock-out on the proteolysis of blood-brain barrier and white matter components after cerebral ischemia. J. Neurosci. 21, 7724-7732 (2001).
Asahi, M. et al. Role for matrix metalloproteinase 9 after focal cerebral ischemia: effects of gene knockout and enzyme inhibition with BB-94. J. Cereb. Blood Flow Metab. 20, 1681-1689 (2000).
Bean, B. P. Neurophysiology: stressful pacemaking Nature 447, 1059-1060 (2007).
Bean, B. P. Nitrendipine block of cardiac calcium channels: high-affinity binding to the inactivated state. Proc. Natl. Acad. Sci. USA 81, 6388-6392 (1984).
Beckman, J. S., Beckman, T. W., Chen, J., Marshall, P. A. & Freeman, B. A. Apparent hydroxyl radical production by peroxynitrite: implications for endothelial injury from nitric oxide and superoxide. Proc. Natl. Acad. Sci. USA 87, 1620-1624 (1990).
Berliocchi, L. et al. Botulinum neurotoxin C initiates two different programs for neurite degeneration and neuronal apoptosis. J. Cell Biol. 168, 607-618 (2005).
Bernaudin, M. et al. A potential role for erythropoietin in focal permanent cerebral ischemia in mice. J. Cereb. Blood Flow Metab. 19, 643-651 (1999).
Bernaudin, M. et al. Neurons and astrocytes express EPO mRNA: oxygen-sensing mechanisms that involve the redox-state of the brain. Glia 30, 271-278 (2000).
Betarbet, R., “Chronic systemic pesticide exposure reproduces features of Parkinson's disease”, Nature Neurosci., 3, 1301-1306, 2000.
Blanpied, T. A., Boekman, F. A., Aizenman, E. & Johnson, J. W. Trapping channel block of NMDAactivated responses by amantadine and memantine. J. Neurophysiol. 77, 309-323 (1997).
Blanpied, T. A., Clarke, R. J. & Johnson, J. W. Amantadine inhibits NMDA receptors by accelerating channel closure during channel block. J. Neurosci. 25, 3312-3322 (2005).
Bonfoco, E., Krainc, D., Ankarcrona, M., Nicotera, P. & Lipton, S. A. Apoptosis and necrosis: two distinct events induced respectively by mild and intense insults with NMDA or nitric oxide/superoxide in cortical cell cultures. Proc. Natl. Acad. Sci. USA 92, 7162-7166 (1995).
Bormann, J. Memantine is a potent blocker of N-methyl-D-aspartate (NMDA) receptor channels. Eur. J. Pharmacol. 166, 591-592 (1989).
Bredt, D. S. et al. Cloned and expressed nitric oxide synthase structurally resembles cytochrome P-450 reductase. Nature 351, 714-718 (1991).
Bresink, I. et al. Effects of memantine on recombinant rat NMDA receptors expressed in HEK 293 cells. Br. J. Pharmacol 119, 195-204 (1996).
Brines, M. & Cerami, A. Emerging biological roles for erythropoietin in the nervous system. Nature Rev. Neurosci. 6, 484-494 (2005).
Bruick, R. K. & McKnight, S. L. A conserved family of prolyl-4-hydroxylases that modify HIF. Science 294, 1337-1340 (2001).
Budd, S. L., Tenneti, L., Lishnak, T. & Lipton, S. A. Mitochondrial and extramitochondrial apoptotic signaling pathways in cerebrocortical neurons. Proc. Natl. Acad. Sci. USA 97, 6161-6166 (2000).
Butterworth, J. F. & Strichartz, G. R. Molecular mechanisms of local anesthesia: a review. Anesthesiology 72, 711-734 (1990).
Calfon, M. et al. IRE1 couples endoplasmic reticulum load to secretory capacity by processing the XBP-1 mRNA. Nature 415, 92-96 (2002).
Campbell, I. L. & Pagenstecher, A. Matrix metalloproteinases and their inhibitors in the nervous system: the good, the bad and the enigmatic. Trends Neurosci. 22, 285-287 (1999).
Chan, C. S. et al. ‘Rejuvenation’ protects neurons in mouse models of Parkinson's disease. Nature 447, 1081-1086 (2007).
Chatterton, J. E. et al. Excitatory glycine receptors containing the NR3 family of NMDA receptor subunits. Nature 415, 793-798 (2002).
Chen, H.-S. V. & Lipton, S. A. in Post Genomic Drug Discovery Research (ed.
Huang, Z.) (John Wiley & Sons, Hoboken, N.J.).
Chen, H.-S. V. & Lipton, S. A. Mechanism of memantine block of NMDA-activated channels in rat retinal ganglion cells: uncompetitive antagonism. J. Physiol. 499, 27-46 (1997).
Chen, H.-S. V. & Lipton, S. A. Pharmacological implications of two distinct mechanisms of interaction of memantine with NMDA-gated channels. J. Pharmacol. Exp. Ther. 314, 961-971 (2005).
Chen, H.-S. V. & Lipton, S. A. The chemical biology of clinically tolerated NMDA receptor antagonists. J. Neurochem. 97, 1611-1626 (2006).
Chen, H.-S. V. et al. J. Neurosci. 12, 4427-4436 (1992).
Chen, H.-S. V. et al. Neuroprotective concentrations of the NMDA open-channel blocker memantine are effective without cytoplasmic vacuolation following post-ischemic administration and do not block maze learning or LTP. Neuroscience 86, 1121-1132 (1998).
Chen, H.-S. V. et al. Open-channel block of N-methyl-D-aspartate (NMDA) responses by memantine: therapeutic advantage against NMDA receptor-mediated neurotoxicity. J. Neurosci. 12, 4427-4436 (1992).
Cho, D.-H., Nakamura, T., Fang, J., Cieplak, P., Godzik, A., Gu, Z. & Lipton, S. A. S-Nitrosylation of Drp1 mediates β-amyloid-related mitochondrial fission and neuronal injury. Science 324, 102-105 (2009).
Choi, D. W. Glutamate neurotoxicity and diseases of the nervous system. Neuron 1, 623-634 (1988).
Meldrum, B. & Garthwaite, J. Excitatory amino acid neurotoxicity and neurodegenerative disease. Trends Pharmacol. Sci. 11, 379-387 (1990).
Choi, Y. B. et al. Molecular basis of NMDA receptor-coupled ion channel modulation by S-nitrosylation. Nature Neurosci. 3, 15-21 (2000).
Chung, K. K. S-Nitrosylation of parkin regulates ubiquitination and compromises parkin's protective function. Science, 304, 1328-1331 (2004).
Conn, K. J. et al. Identification of the protein disulfide isomerase family member PDIp in experimental Parkinson's disease and Lewy body pathology. Brain Res., 1022, 164-172 (2004).
Das, S. et al. Increased NMDA current and spine density in mice lacking the NMDA receptor subunit NR3A. Nature 393, 377-381 (1998).
Dawson, V. L., Dawson, T. M., Bartley, D. A., Uhl, G. R. & Snyder, S. H. Mechanisms of nitric oxide-mediated neurotoxicity in primary brain cultures. J. Neurosci 13, 2651-2661 (1993).
Dawson, V. L., Dawson, T. M., London, E. D., Bredt, D. S. & Snyder, S. H. Nitric oxide mediates glutamate neurotoxicity in primary cortical cultures. Proc. Natl. Acad. Sci. USA 88, 6368-6371 (1991).
De Felice, F. G. et al. Aβ oligomers induce neuronal oxidative stress through an N-methyl-D-aspartate receptor-dependent mechanism that is blocked by the Alzheimer drug memantine. J. Biol. Chem. 282, 11590-11601 (2007).
Digicaylioglu, M. & Lipton, S. A. Erythropoietin-mediated neuroprotection involves cross-talk between Jak2 and NF-κB signaling cascades. Nature 412, 641-647 (2001).
Digicaylioglu, M. et al. Localization of specific erythropoietin binding sites in defined areas of the mouse brain. Proc. Natl. Acad. Sci. USA 92, 3717-3720 (1995).
Dreyer, E. B. & Grosskreutz, C. L. Excitatory mechanisms in retinal ganglion cell death in primary open angle glaucoma (POAG). Clin. Neurosci. 4, 270-273 (1997).
Dreyer, E. B. & Lipton, S. A. New perspectives on glaucoma. JAMA 281, 306-308 (1999).
Dreyer, E. B., Zhang, D. & Lipton, S. A. Transciptional or translational inhibition blocks low dose NMDA mediated cell death. NeuroReport 6, 942-944 (1995).
Edman, J. C., Ellis, L., Blacher, R. W., Roth, R. A., Rutter, W. J., “Sequence of protein disulphide isomerase and implications of its relationship to thioredoxin” Nature, 1985, 317, 267-270, 1985.
Ehrenreich, H. et al. Erythropoietin therapy for acute stroke is both safe and beneficial. Mol. Med. 8, 495-505 (2002).
Ellgaard, L., Molinari, M., Helenius, A., “Setting the standards: quality control in the secretory pathway”, Science, 286, 1882-1888, 1999.
Finch, C. A. Erythropoiesis, erythropoietin, and iron. Blood 60, 1241-1246 (1982).
Garden, G. A. et al. Caspase cascades in human immunodeficiency virus-associated neurodegeneration. J. Neurosci. 22, 4015-4024 (2002).
Gasche, Y. et al. Early appearance of activated matrix metalloproteinase-9 after focal cerebral ischemia in mice: a possible role in blood-brain barrier dysfunction. J. Cereb. Blood Flow Metab. 19, 1020-1028 (1999).
Gilbert, H. F., “Protein disulfide isomerase”, Methods Enzymol., 290, 26-50, (1998).
Gu, Z. et al. A highly specific inhibitor of matrix metalloproteinase-9 rescues laminin from proteolysis and neurons from apoptosis in transient focal cerebral ischemia. J. Neurosci. 25, 6401-6408 (2005).
Gu, Z. et al. S-Nitrosylation of matrix metalloproteinases: signaling pathway to neuronal cell death. Science 297, 1186-1190 (2002).
Haendeler, J., “Redox regulatory and anti-apoptotic functions of thioredoxin depend on S-nitrosylation at cysteine 69”, Nature Cell Biol., 4, 743-749, 2002.
Hara, M. R. et al. S-nitrosylated GAPDH initiates apoptotic cell death by nuclear translocation following Siahl binding. Nature Cell. Biol. 7, 665-674 (2005).
Hardingham, G. E., Fukunaga, Y. & Bading, H. Extrasynaptic NMDARs oppose synaptic NMDARs by triggering CREB shut-off and cell death pathways. Nature Neurosci. 5, 405-414 (2002).
Haynes, C. M., Titus, E. A., “Degradation of misfolded proteins prevents ER-derived oxidative stress and cell death”, Mol. Cell, 15, 767-776, 2004.
Heo, J. H. et al. Matrix metalloproteinases increase very early during experimental focal cerebral ischemia. J. Cereb. Blood Flow Metab. 19, 624-633 (1999).
Hess, D. T., Matsumoto, A., Kim, S. O., Marshall, H. E. & Stamler, J. S. Protein S-nitrosylation: purview and parameters. Nature Rev. Mol. Cell. Biol. 6, 150-166 (2005).
Hickenbottom, S. L. & Grotta, J. Neuroprotective therapy. Semin. Neurol. 18, 485-492 (1998).
Horibe, T. et al. Different contributions of the three COX motifs of human protein-disulfide isomerase-related protein to isomerase activity and oxidative refolding. J. Biol. Chem. 279, 4604-4611 (2004).
Hu, B. R., Martone, M. E., Jones, C. L., Liu, C. L., “Protein aggregation after transient cerebral ischemia”, 20, 3191-3199, 2000.
Huettner, J. E. & Bean, B. P. Block of N-methyl-D-aspartate-activated current by the anticonvulsant MK-801: selective binding to open channels. Proc. Natl. Acad. Sci. USA 85, 1307-1311 (1988).
Ikejiri, M. et al. Potent mechanism-based inhibitors for matrix metalloproteinases. J. Biol. Chem. 280, 33992-34002 (2005).
Imai, Y. An unfolded putative transmembrane polypeptide, which can lead to endoplasmic reticulum stress, is a substrate of Parkin. Cell 105, 891-902 (2001).
Itoh, K., Tong, K. I. & Yamamoto, M. Molecular mechanism activating Nrf2-Keap1 pathway in regulation of adaptive response to electrophiles. Free Radic. Biol. Med. 36, 1208-1213 (2004).
Jaakkola, P. et al. Targeting of HIF-α to the von Hippel-Lindau ubiquitylation complex by O2-regulated prolyl hydroxylation. Science 292, 468-472 (2001).
Jaffrey, S. R., Erdjument-Bromage, H., Ferris, C. D., Tempst, P., Snyder, S. H. Protein S-nitrosylation: a physiological signal for neuronal nitric oxide. Nature Cell Biol., 3, 193-197 (2001).
Jelkmann, W. Erythropoietin: structure, control of production, and function. Physiol. Rev. 72, 449-489 (1992).
Jian Liu, K. & Rosenberg, G. A. Matrix metalloproteinases and free radicals in cerebral ischemia. Free Radic. Biol. Med. 39, 71-80 (2005).
Johnson, J. W. & Ascher, P. Glycine potentiates the NMDA response in cultured mouse brain neurons. Nature 325, 529-531 (1987).
Káradóttir, R., Cavelier, P., Bergersen, L. H. & Atwell, D. NMDA receptors in oligodendrocyte physiology and pathology. Nature (in the press).
Karschin, A., Aizenman, E. & Lipton, S. A. The interaction of agonists and noncompetitive antagonists at the excitatory amino acid receptors in rat retinal ganglion cells in vitro. J. Neurosci. 8, 2895-2906 (1988).
Kask K, Zamanillo D, Rozov A, Brnashev N, Sprengel R, Seeburg P H. The AMPA receptor subunit GluR-B in its Q/R site-unedited form is not essential for brain development and function Proc Natl Acad Sci USA 95, 13777-13782 (1998).
Kaufman, R. J. Stress signaling from the lumen of the endoplasmic reticulum: coordination of gene transcriptional and translational controls. Genes Dev. 13, 1211-1233 (1999).
Kaul, M., Garden, G. A. & Lipton, S. A. Pathways to neuronal injury and apoptosis in HIV-associated dementia. Nature 410, 988-994 (2001).
Kemp J A & McKernan R M. NMDA receptor pathways as drug targets. Nat. Neurosci. 5, 1039-1042 (2002).
Kemp, J. A., Kew, J. N. & Gill, D. L. in Handbook of Experimental Pharmacology (eds. Jonas, P. & Monyer, H.) 495-527 (Springer, Berlin, 1999).
Kim, W. K. et al. Attenuation of NMDA receptor activity and neurotoxicity by nitroxyl anion, NO—. Neuron 24, 461-469 (1999).
Kleckner, N. W. & Dingledine, R. Requirement for glycine in activation of NMDA-receptors expressed in Xenopus oocytes. Science 241, 835-837 (1988).
Ko, H. S., Uehara, T., Nomura, Y. Role of ubiquilin associated with protein-disulfide isomerase in the endoplasmic reticulum in stress-induced apoptotic cell death. J. Biol. Chem. 277, 35386-35392 (2002).
Koroshetz, W. J. & Moskowitz, M. A. Emerging treatments for stroke in humans. Trends Pharmacol. Sci. 17, 227-233 (1996).
Kraft, A. D., Johnson, D. A. & Johnson, J. A. Nuclear factor E2-related factor 2-dependent antioxidant response element activation by tert-butylhydroquinone and sulforaphane occurring preferentially in astrocytes conditions neurons against oxidative insult. J. Neurosci. 24, 1101-1112 (2004).
Kwak S, Kawahara Y. Deficient RNA editing of GluR2 and neuronal death in amyotrophic lateral sclerosis. J Mol Med 83, 110-120 (2005).
Lacor, P. N. et al. Aβ oligomer-induced aberrations in synapse composition, shape, and density provide a molecular basis for loss of connectivity in Alzheimer's disease. J. Neurosci. 27, 796-807 (2007).
Lando, D., Peet, D. J., Whelan, D. A., Gorman, J. J. & Whitelaw, M. L. Asparagine hydroxylation of the HIF transactivation domain a hypoxic switch. Science 295, 858-861 (2002).
Le, D. A. & Lipton, S. A. Potential and current use of N-methyl-D-aspartate (NMDA) receptor antagonists in diseases of aging. Drugs Aging 18, 717-724 (2001).
Lees, K. R. et al. Glycine antagonist (gavestinel) in neuroprotection (GAIN International) in patients with acute stroke: a randomised controlled trial. GAIN International Investigators. Lancet 355, 1949-1954 (2000).
Lei, S. Z. et al. Effect of nitric oxide production on the redox modulatory site of the NMDA receptor-channel complex. Neuron 8, 1087-1099 (1992).
Lipton S A. Pathologically activated therapeutics for neuroprotection. Nat. Rev Neurosci. 8, 803-808 (2007).
Lipton, S. A. & Chen, H.-S. Paradigm shift in neuroprotective drug development: clinically tolerated NMDA receptor inhibition by memantine. Cell Death Differ. 11, 18-20 (2004).
Lipton, S. A. & Gendelman, H. E. Dementia associated with the acquired immunodeficiency syndrome. N. Engl. J. Med. 332, 934-940 (1995).
Lipton, S. A. & Nicotera, P. Calcium, free radicals and excitotoxins in neuronal apoptosis. Cell Calcium 23, 165-171 (1998).
Lipton, S. A. & Rosenberg, P. A. Mechanisms of disease: Excitatory amino acids as a final common pathway for neurologic disorders. N. Engl. J. Med. 330, 613-622 (1994).
Lipton, S. A. & Wang, Y. F. in Pharmacology of Cerebral Ischemia (ed. Krieglstein, J.) 183-191 (Medpharm Scientific, Stuttgart, 1996).
Lipton, S. A. Concepts: turning down, but not off. Nature 428, 473 (2004).
Lipton, S. A. Erythropoietin for neurologic protection and diabetic neuropathy. N. Engl. J. Med. 350, 2516-2517 (2004).
Lipton, S. A. et al. A redox-based mechanism for the neuroprotective and neurodestructive effects of nitric oxide and related nitroso-compounds. Nature 364, 626-632 (1993).
Lipton, S. A. et al. Cysteine regulation of protein function—as exemplified by NMDA-receptor modulation. Trends Neurosci. 25, 474-480 (2002).
Lipton, S. A. Memantine prevents HIV coat proteininduced neuronal injury in vitro. Neurology 42, 1403-1405 (1992).
Lipton, S. A. Molecular mechanisms of trauma-induced neuronal degeneration. Curr. Opin. Neurol. Neurosurg. 6, 588-596 (1993).
Lipton, S. A. NMDA receptors, glial cells, and clinical medicine. Neuron 50, 9-11 (2006).
Lipton, S. A. Paradigm shift in neuroprotection by NMDA receptor blockade: memantine and beyond. Nature Rev. Drug Discov. 5, 160-170 (2006).
Lipton, S. A. Prospects for clinically tolerated NMDA antagonists: open-channel blockers and alternative redox states of nitric oxide. Trends Neurosci 16, 527-532 (1993).
Lipton, S. A., Rayudu, P. V., Choi, Y.-B., Sucher, N. J. & Chen, H.-S. V. in Prog Brain Res. (eds Mize, V., Dawson, T. M., Dawson, M. & Friedlander, M.) 73-82 (Elsevier, Amsterdam, 1998).
Lipton, S. A., Rosenberg, P. A. Excitatory amino acids as a final common pathway for neurologic disorders. N. Engl. J. Med. 330, 613-622 (1994).
Lucas, D. R. & Newhouse, J. P. The toxic effect of sodium L-glutamate on the inner layers of the retina. Arch. Ophathalmol. 58, 193-201 (1957).
Lukes, A., Mun-Bryce, S., Lukes, M. & Rosenberg, G. A. Extracellular matrix degradation by metalloproteinases and central nervous system diseases. Mol. Neurobiol. 19, 267-284 (1999).
Lutsep, H. L. & Clark, W. M. Neuroprotection in acute ischaemic stroke. Current status and future potential. Drugs R & D 1, 3-8 (1999).
Maiese, K., Li, F. & Chong, Z. Z. New avenues of exploration for erythropoietin. JAMA 293, 90-95 (2005).
Martin, J. et al. Chaperonin-mediated protein folding at the surface of groEL through a ‘molten globule’-like intermediate. Nature 352, 36-42 (1991).
Masuda, S. et al. Functional erythropoietin receptor of the cells with neural characteristics. Comparison with receptor properties of erythroid cells. J. Biol. Chem. 268, 11208-11216 (1993).
Mattson, M. P. Pathways towards and away from Alzheimer's disease. Nature 430, 631-639 (2004).
Mayer, M. L., Westbrook, G. L. & Guthrie, P. B. Voltage-dependent block by Mg²⁺ of NMDA responses in spinal cord neurones. Nature 309, 261-263 (1984).
McBain, C. J. & Mayer, M. L. N-Methyl-D-aspartic acid receptor structure and function. Physiol. Rev. 74, 723-760 (1994).
McKercher, S. R., Nakamura, T., Lipton, S. A. From Reactive Oxygen and Nitrogen Species to Therapy. In: Encyclopedia of Life Sciences: Cell Death. Melino G, Vaux D, eds, John Wiley & Sons, 2010, pp. 262-272.
Meguro, H. et al. Functional characterization of a heteromeric NMDA receptor channel expressed from cloned cDNAs. Nature 357, 70-74 (1992).
Meldrum, B. & Garthwaite, J. Excitatory amino acid neurotoxicity and neurodegenerative disease. Trends Pharmacol. Sci. 11, 379-387 (1990).
Micu, I. et al. NMDA receptors mediate calcium accumulation in central nervous system myelin during chemical ischaemia. Nature 439, 988-992 (2006).
Montaner, J. et al. Matrix metalloproteinase expression after human cardioembolic stroke: temporal profile and relation to neurological impairment. Stroke 32, 1759-1766 (2001).
Monyer, H. et al. Heteromeric NMDA receptors: molecular and functional distinction of subtypes. Science 256, 1217-1221 (1992).
Mori, H., Masaki, H., Yamakura, T. & Mishina, M. Identification by mutagenesis of a Mg²⁺-block site of the NMDA receptor channel. Nature 358, 673-675 (1992).
Morishita, E., Masuda, S., Nagao, M., Yasuda, Y. & Sasaki, R. Erythropoietin receptor is expressed in rat hippocampal and cerebral cortical neurons, and erythropoietin prevents in vitro glutamate-induced neuronal death. Neuroscience 76, 105-116 (1997).
Mothet, J. P. et al. D-serine is an endogenous ligand for the glycine site of the N-methyl-D-aspartate receptor. Proc. Natl. Acad. Sci. USA 97, 4926-4931 (2000).
Murakami, T., “Pael-R is accumulated in Lewy bodies of Parkinson's disease”, Ann. Neurol., 55, 439-442, 2004.
Nadal M. S et al., The CD26-related dipeptidyl aminopeptidase-like protein DPPX is a critical component of Neuronal A-type K⁺ channels. Neuron 37, 449-461 (2003).
Nakanishi, S. Molecular diversity of glutamate receptors and implications for brain function. Science 258, 597-603 (1992).
Nakanishi, N., Tu, S., Shin, Y., Cui, J., Kurokawa, T., Zhang, D., Chen, H.-S. V., Tong, G. & Lipton, S. A. Neuroprotection by the NR3A subunit of the NMDA receptor. J. Neurosci. 29, 5260-5265 (2009).
Naskar, R., Vorwerk, C. K. & Dreyer, E. B. Saving the nerve from glaucoma: memantine to caspaces. Semin. Ophthalmol. 14, 152-158 (1999).
Navia, B. A., Yiannoutsos, C., Ellis, R., Schifitto, G., Nath, A., Shriver, S., Millar, L. & Lipton, S. A. Memantine may prevent further cognitive decline in subjects with AIDS Dementia Complex with detectable CSF HIV RNA. Neurology 64 (Suppl. 1), A247-A238 (2005).
Nishikawa, A., “Novel function of PS2V: change in conformation of tau proteins”, Biochem. Biophys. Res. Commun., 318, 435-438, 2004.
Nowak, L., Bregestovski, P., Ascher, P., Herbet, A. & Prochiantz, A. Magnesium gates glutamate-activated channels in mouse central neurones. Nature 307, 462-465 (1984).
Okamoto S-i, Pouladi M, Talantova M, Yao D, Xia P, Ehrnhoefer D E, Zaidi R, Clemente A, Kaul M, Graham R K, Zhang D, Chen H-S V, Tong G, Hayden M R, Lipton S A. Balance between synaptic versus extrasynaptic NMDA receptor activity influences inclusions and neurotoxicity of mutant huntingtin. Nature Med. 15, 1407-1413 (2009).
Okamoto, S. et al. Dominant-interfering forms of MEF2 generated by caspase cleavage contribute to NMDA-induced neuronal apoptosis. Proc. Natl. Acad. Sci. USA 99, 3974-3979 (2002).
Olney, J. W. & Ho, O. L. Brain damage in infant mice following oral intake of glutamate, aspartate or cysteine. Nature 227, 609-611 (1970).
Olney, J. W. Glutamate-induced retinal degeneration in neonatal mice. Electron microscopy of the acutely evolving lesion. J. Neuropathol. Exp. Neurol. 28, 455-474 (1969).
Orgogozo, J. M., Rigaud, A. S., Stoffler, A., Mobius, H. J. & Forette, F. Efficacy and safety of memantine in patients with mild to moderate vascular dementia: a randomized, placebo-controlled trial (MMM 300). Stroke 33, 1834-1839 (2002).
Osborne, N. N. Memantine reduces alterations to the mammalian retina, in situ, induced by ischemia. Vis. Neurosci. 16, 45-52 (1999).
Palmer, G. C. Neuroprotection by NMDA receptor antagonists in a variety of neuropathologies. Curr. Drug Targets 2, 241-271 (2001).
Parsons, C. G., Danysz, W. & Quack, G. Memantine is a clinically well tolerated N-methyl-D-aspartate (NMDA) receptor antagonist—a review of preclinical data. Neuropharmacology 38, 735-767 (1999).
Patil, C., Walter, P., “Intracellular signaling from the endoplasmic reticulum to the nucleus: the unfolded protein response in yeast and mammals”, Curr. Opin. Cell Biol., 13, 349-355, 2001.
Pellegrini, J. W. & Lipton, S. A. Delayed administration of memantine prevents N-methyl-D-aspartate receptor mediated neurotoxicity. Ann. Neurol. 33, 403-407 (1993).
Quigley, H. A. et al. Retinal ganglion cell death in experimental glaucoma and after axotomy occurs by apoptosis. Invest. Ophthalmol. Vis. Sci. 36, 774-786 (1995).
Rammes, G., Rupprecht, R., Ferrari, U., Zieglgansberger, W. & Parsons, C. G. The N-methyl-D-aspartate receptor channel blockers memantine, MRZ 2/579 and other amino-alkyl-cyclohexanes antagonise 5-HT(3) receptor currents in cultured HEK-293 and N1E-115 cell systems in a non-competitive manner. Neurosci. Lett. 306, 81-84 (2001).
Rao, R. V., Bredesen, D. E., “Misfolded proteins, endoplasmic reticulum stress and neurodeneration”, Curr. Opin. Cell Biol., 16, 653-662, 2004.
Reisberg, B. et al. Memantine in moderate-to-severe Alzheimer's disease. N. Engl. J. Med. 348, 1333-1341 (2003).
Reiser, G., Binmoller, F. J. & Koch, R. Memantine (1-amino-3,5-dimethyladamantane) blocks the serotonin-induced depolarization response in a neuronal cell line. Brain Res. 443, 338-344 (1988).
Rogawski, M. A. & Wenk, G. L. The neuropharmacological basis for the use of memantine in the treatment of Alzheimer's disease. CNS Drug Rev. 9, 275-308 (2003).
Rogawski, M. A. Low affinity channel blocking (uncompetitive) NMDA receptor antagonists as therapeutic agents—toward an understanding of their favorable tolerability. Amino Acids 19, 133-149 (2000).
Romanic, A. M., White, R. F., Arleth, A. J., Ohlstein, E. H. & Barone, F. C. Matrix metalloproteinase expression increases after cerebral focal ischemia in rats: inhibition of matrix metalloproteinase-9 reduces infarct size. Stroke 29, 1020-1030 (1998).
Rothman, S. M. & Olney, J. W. Excitotoxicity and the NMDA receptor—still lethal after eight years. Trends Neurosci. 18, 57-58 (1995).
Rothman, S. M. & Olney, J. W. Excitotoxicity and the NMDA receptor. Trends Neurosci. 10, 299-302 (1987).
Ryu, E. J., “Endoplasmic reticulum stress and the unfolded protein response in cellular models of Parkinson's disease”, J. Neurosci., 22, 10690-10698, 2002.
Sacco, R. L. et al. Glycine antagonist in neuroprotection for patients with acute stroke: GAIN Americas: a randomized controlled trial. JAMA 285, 1719-1728 (2001).
Salter, M. & Fern, R. NMDA receptors are expressed in developing oligodendrocyte processes and mediate injury. Nature (in the press).
Satoh, T. & Lipton, S. A. Redox regulation of neuronal survival mediated by electrophilic compounds. Trends Neurosci 30, 37-45 (2007).
Satoh, T. et al. Activation of the Keap1/Nrf2 pathway for neuroprotection by electrophilic phase II inducers. Proc. Natl. Acad. Sci. USA 103, 768-773 (2006).
Seif el Nasr, M., Perucher, B., Rossberg, C., Mennel, H.-D. & Krieglstein, J. Neuroprotective effect of memantine demonstrated in vivo and in vitro. Eur. J Pharmacol. 185, 19-24 (1990).
Selkoe, D. J. Alzheimer's disease: genes, proteins, and therapy. Physiol. Rev. 81, 741-766 (2001).
Shankar, G. M. et al. Natural oligomers of the Alzheimer amyloid-β protein induce reversible synapse loss by modulating an NMDA-type glutamate receptor-dependent signaling pathway. J. Neurosci. 27, 2866-2875 (2007).
Shih, A. Y., Li, P. & Murphy, T. H. A small-molecule-inducible Nrf2-mediated antioxidant response provides effective prophylaxis against cerebral ischemia in vivo. J. Neurosci. 25, 10321-10335 (2005).
Shleper, M., Kartvelishvily, E. & Wolosker, H. D-serine is the dominant endogenous coagonist for NMDA receptor neurotoxicity in organotypic hippocampal slices. J. Neurosci. 25, 9413-9417 (2005).
Sliskovic, I, Raturi, A., Mutus, B., “Characterization of the S-denitrosation activity of protein disulfide isomerase”, J. Biol. Chem., 280, 8733-8741, 2005.
Sobolevsky A I, Rosconi M P, Gouaux E. X-ray structure, symmetry and mechanism of an AMPA-subtype glutamate receptor. Nature 462, 745-756 (2009).
Sobolevsky, A. I., Koshelev, S. G. & Khodorov, B. I. Interaction of memantine and amantadine with agonist-unbound NMDA-receptor channels in acutely isolated rat hippocampal neurons. J. Physiol. (Lond.) 512, 47-60 (1998).
Song, J. L., Wang, C. C., “Chaperone-like activity of protein disulfide-isomerase in the refolding of rhodanese”, Eur. J. Biochem, 231,312-316, 1995.
Stamler, J. S., Redox signaling: nitrosylation and related target interactions of nitric oxide”, Cell, 78, 931-936, 1994.
Stamler, J. S., Singel, D. J. & Loscalzo, J. Biochemistry of nitric oxide and its redox-activated forms. Science 258, 1898-1902 (1992).
Stern-Bach, Y., Bettler, B., Hartley, M., Sheppard, P. O., O'Hara, P. J. & Heinemann, S. F. Agonist selectivity of glutamate receptors is specified by two domains structurally related to bacterial amino acid-binding proteins. Neuron 13, 1345-1357 (1994).
Sucher, N. J. et al. Developmental and regional expression pattern of a novel NMDA receptor-like subunit (NMDAR-L) in the rodent brain. J. Neurosci. 15, 6509-6520 (1995).
Sucher, N. J., Lipton, S. A. & Dreyer, E. B. Molecular basis of glutamate toxicity in retinal ganglion cells. Vision Res. 37, 3483-3493 (1997).
Takahashi, H. et al. Hypoxia enhances S-nitrosylation-mediated NMDA receptor inhibition via a thiol oxygen sensor motif. Neuron 53, 53-64 (2007).
Tanaka, S., Uehara, T., Nomura, Y. Up-regulation of protein-disulfide isomerase in response to hypoxia/brain ischemia and its protective effect against apoptotic cell death. J. Biol. Chem., 275, 10388-10393 (2000).
Tariot, P. N. et al. Memantine treatment in patients with moderate to severe Alzheimer disease already receiving donepezil: a randomized controlled trial. JAMA 291, 317-324 (2004).
Tenneti, L., D'Emilia, D. M., Troy, C. M. & Lipton, S. A. Role of caspases in N-methyl-D-aspartate-induced apoptosis of cerebrocortical neurons. J. Neurochem. 71, 946-959 (1998).
Uehara, T., Nakamura, T., Yao, D., Shi, Z.-Q., Gu, Z., Masliah, E., Nomura, Y. & Lipton, S. A. S-Nitrosylation of protein-disulphide isomerase links protein misfolding to neurodegeneration. Nature 441, 513-517 (2006).
Vorwerk, C. K. et al. Chronic low dose glutamate is toxic to retinal ganglion cells: toxicity blocked by memantine. Invest. Ophthalmol. Vis. Sci. 37, 1618-1624 (1996).
Vuori, K., Myllyla, R., Pihlajaniemi, T., Kivirikko, K. I., “Expression and site-directed mutagenesis of human protein disulfide isomerase in Escherichia coli. This multifunctional polypeptide has two independently acting catalytic sites for the isomerase activity”, J. Biol. Chem, 267, 7211-7214, 1992.
Wada, A., Takahashi, H., Lipton, S. A. & Chen, H.-S. V. NR3A modulates the outer vestibule of the “NMDA” receptor channel. J. Neurosci. 26, 13156-13166 (2006).
Wang, H. et al. Apoptosis-inducing factor substitutes for caspase executioners in NMDA triggered excitotoxic neuronal death. J. Neurosci. 24, 10963-10973 (2004).
Winblad, B. & Poritis, N. Memantine in severe dementia: Results of the 9M-best study (benefit and efficacy in severely demented patients during treatment with memantine). Int. J. Geriat. Psych. 14, 135-146 (1999).
Wolosker, H., Blackshaw, S. & Snyder, S. H. Serine racemase: a glial enzyme synthesizing D-serine to regulate glutamate-N-methyl-D-aspartate neurotransmission. Proc. Natl. Acad. Sci. USA 96, 13409-13414 (1999).
Wong, H. K. et al. Temporal and regional expression of NMDA receptor subunit NR3A in the mammalian brain. J. Comp. Neurol. 450, 303-317 (2002).
Wu, P. H., Moron, M. & Barraco, R. Organic calcium channel blockers enhance [3H]purine release from rat brain cortical synaptosomes. Neurochem. Res. 9, 1019-1031.
Yang, Y., Estrada, E. Y., Thompson, J. F., Liu, W. & Rosenberg, G. A. Matrix metalloproteinase-mediated disruption of tight junction proteins in cerebral vessels is reversed by synthetic matrix metalloproteinase inhibitor in focal ischemia in rat. J. Cereb. Blood Flow Metab. 27, 697-709 (2007).
Yao, D., “Nitrosative stress linked to sporadic Parkinson's disease: S-nitrosylation of parkin regulates its E3 ubiquitin ligase activity”, Proc. Natl. Acad. Sci. USA, 101, 10810-10814, 2004.
Yong, V. W. Metalloproteinases: mediators of pathology and regeneration in the CNS. Nature Rev. Neurosci. 6, 931-944 (2005).
Yong, V. W., Power, C., Forsyth, P. & Edwards, D. R. Metalloproteinases in biology and pathology of the nervous system. Nature Rev. Neurosci. 2, 502-511 (2001).
Yoshida, H., Matsui, T., Yamamoto, A., Okada, T. & Mori, K. XBP1 mRNA is induced by ATF6 and spliced by IRE1 in response to ER stress to produce a highly active transcription factor. Cell 107, 881-891 (2001).
Yun, H.-Y., Gonzalez-Zulueta, M., Dawson, V. L. & Dawson, T. M. Nitric oxide mediates N-methyl-D-aspartate receptor-induced activation of p21ras. Proc. Natl. Acad. Sci. USA 95, 5773-5778 (1998).
Xia P, Chen H-S V, Zhang D, Lipton S A. Memantine preferentially blocks extrasynaptic over synaptic NMDA receptor currents in hippocampal autapses, J. Neurosci. 30, 11246-11250 (2010).
Zeevalk, G. D. & Nicklas, W. J. Evidence that the loss of the voltage-dependent Mg2+ block of the N-methyl-D-aspartate receptor underlies receptor activation during inhibition of neuronal metabolism. J. Neurochem. 59, 1211-1220 (1992).
Zhao, B. Q. et al. Role of matrix metalloproteinases in delayed cortical responses after stroke. Nature Med. 12, 441-445 (2006).

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a pathologically-activated compound” includes a plurality of such pathologically-activated compounds, reference to “the pathologically-activated compound” is a reference to one or more pathologically-activated compounds and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps.

Those skilled in the art will recognize, or be able to ascertain using no more than routine experimentation, many equivalents to the specific embodiments of the method and compositions described herein. Such equivalents are intended to be encompassed by the following claims.

1. A method of identifying a pathologically-activated targeting compound, the method comprising determining if a test compound binds uncompetitively to a target molecule, wherein if the test compound binds uncompetitively to the target molecule then the test compound is identified as a pathologically-activated targeting compound.

2. The method of claim 1 further comprising determining if the test compound is an uncompetitive inhibitor of the target molecule, wherein if the test compound is an uncompetitive inhibitor of the target molecule then the test compound is identified as a pathologically-activated compound.

3. The method of claim 2, wherein determining if a test compound binds uncompetitively to the target molecule and determining if the test compound is an uncompetitive inhibitor of the target molecule are accomplished in different assays.

4. The method of claim 2, wherein determining if a test compound binds uncompetitively to the target molecule is performed prior to determining if the test compound is an uncompetitive inhibitor of the target molecule.

5. The method of claim 2, wherein determining if the test compound is an uncompetitive inhibitor of the target molecule is performed prior to determining if a test compound binds uncompetitively to the target molecule.

6. The method of claim 2, wherein determining if a test compound binds uncompetitively to the target molecule and determining if the test compound is an uncompetitive inhibitor of the target molecule are accomplished in the same assay.

7. The method of claim 1, wherein a nitro group is linked to the pathologically-activated targeting compound.

8. The method of claim 1, wherein a redox-activated group is linked to the pathologically-activated targeting compound, wherein the redox-activated group becomes chemically reactive when exposed to oxidizing conditions or the redox-activated group becomes chemically reactive when exposed to reducing conditions.

9. The method of claim 8, wherein the redox-activated group becomes chemically reactive when exposed to oxidizing conditions.

10. The method of claim 1 further comprising determining if the test compound has a fast off-rate for the target molecule.

11. The method of claim 10, wherein the off-rate is a fast off-rate if the off-rate has a ratio of 2:1 to the on-rate.

12. The method of claim 1, wherein determining if the test compound binds uncompetitively to the target molecule is accomplished by steps comprising bringing into contact the target molecule and the test compound and determining if the test compound exhibits uncompetitive binding to the target molecule.

13. The method of claim 2, wherein determining if the test compound is an uncompetitive inhibitor of the target molecule is accomplished by steps comprising bringing into contact the target molecule and the test compound and determining if the test compound exhibits uncompetitive inhibition on the activity of the target molecule.

14. The method of claim 1, wherein determining if the test compound binds uncompetitively to the target molecule is accomplished by comparing the binding of the test molecule in the presence of the same concentration of test compound and different concentrations of a molecule know to bind the target molecule.

15. The method of claim 2, wherein determining if the test compound is an uncompetitive inhibitor of the target molecule is accomplished by comparing the activity of the target molecule in the presence of the same concentration of test compound and different concentrations of an agonist of the target molecule.

16. The method of claim 1, wherein determining if the test compound binds uncompetitively to the target molecule is determined by measuring the ratio of binding to the target molecule in the presence of a concentration of test compound and a first concentration of a molecule known to bind the target molecule compared to binding to the target molecule in the presence of the same concentration of test compound and a second concentration of the molecule known to bind the target molecule, wherein the first concentration of the molecule known to bind the target molecule is greater than the second concentration of the molecule known to bind the target molecule, wherein a ratio of greater than 1 indicates that the test compound binds uncompetitively to the target molecule.

17. The method of claim 2, wherein determining if the test compound is an uncompetitive inhibitor of the target molecule is determined by measuring the ratio of the activity of the target molecule in the presence of a concentration of test compound and a first concentration of an agonist of the target molecule to the activity of the target molecule in the presence of the same concentration of test compound and a second concentration of an agonist of the target molecule, wherein the first concentration of agonist is greater than the second concentration of agonist, wherein a ratio of greater than 1 indicates that the test compound is an uncompetitive inhibitor of the target molecule.

18. The method of claim 1, wherein determining if the test compound binds uncompetitively to the target molecule is accomplished by comparing the binding of the test compound in the presence of the same concentration of test compound and different expression levels of the target molecule.

19. The method of claim 2, wherein determining if the test compound is an uncompetitive inhibitor of the target molecule is accomplished by comparing the activity of the target molecule in the presence of the same concentration of test compound and different expression levels of the target molecule.

20. The method of claim 1, and 19, wherein determining if the test compound binds uncompetitively to the target molecule is determined by measuring the ratio of binding to the target molecule in the presence of a concentration of test compound and a first expression level of the target molecule compared to binding to the target molecule in the presence of the same concentration of test compound and a second expression level of the target molecule, wherein the first expression level of the target molecule is greater than the second expression level of the target molecule, wherein a ratio of greater than 1 indicates that the test compound binds uncompetitively to the target molecule.

21. The method of claim 2, and 20, wherein determining if the test compound is an uncompetitive inhibitor of the target molecule is determined by measuring the ratio of the activity of the target molecule in the presence of a concentration of test compound and a first expression level of the target molecule to the activity of the target molecule in the presence of the same concentration of test compound and a second expression level of the target molecule, wherein the first expression level of the target molecule is greater than the second expression level of the target molecule, wherein a ratio of greater than 1 indicates that the test compound is an uncompetitive inhibitor of the target molecule.

22. The method of claim 1, wherein the test compound is a modified form of a prior test compound, wherein the prior test compound was determined to bind uncompetitively to the target molecule, determined to be an uncompetitive inhibitor of the target molecule, or both.

23. The method of claim 22, wherein the prior test compound is a modified form of a second prior test compound, wherein the second prior test compound was determined to bind uncompetitively to the target molecule, determined to be an uncompetitive inhibitor of the target molecule, or both.

24. The method of claim 22, wherein the prior test compound was produced via multiple iterations of modifying an earlier test compound that was determined to bind uncompetitively to the target molecule, determined to be an uncompetitive inhibitor of the target molecule, or both, and determining if the modified test compound binds uncompetitively to the target molecule, is an uncompetitive inhibitor of the target molecule, or both.

25. The method of claim 22, wherein different iterations and/or different instances of determining if any of the test compounds binds uncompetitively to the target molecules and/or different iterations and/or different instances of determining if any of the test compounds is an uncompetitive inhibitor of the target molecule are accomplished with the same type of assays, different types of assays, or a combination.

26. The method of claim 1, wherein the test molecule has an apparent affinity for the target molecule of about 100 nM or greater.

27. The method of claim 1, wherein the test molecule has an apparent affinity for the target molecule of about 1 μM or greater.

28. The method of claim 1, wherein the test molecule has an apparent affinity for the target molecule of about 10 μM or greater.

29. The method of claim 1, wherein the test molecule has an apparent affinity for the target molecule of about 100 μM or greater.

30. The method of claim 1, wherein the target molecule is a receptor.

31. The method of claim 30, wherein the receptor is a neuroreceptor.

32. The method of claim 30, wherein the receptor is a G-coupled receptor.

33. The method of claim 30, wherein the receptor is a channel receptor.

34. The method of claim 30, wherein the receptor is an N-methyl-D-aspartate (NMDA)-sensitive, AMPA-sensitive, or Kainate-sensitive glutamate receptor.

35. The method of claim 1 further comprising producing the pathologically-activated targeting compound.

36. The method of claim 2 further comprising producing the pathologically-activated compound.

37. The method of claim 1, wherein determining if a test compound binds uncompetitively to a target molecule is performed simultaneously on 96 or more test compounds.

38. The method of claim 2, wherein determining if a test compound is an uncompetitive inhibitor of a target molecule is performed simultaneously on 96 or more test compounds.

39. The method of claim 1, wherein determining if a test compound binds uncompetitively to a target molecule is accomplished using a high throughput screen.

40. The method of claim 2, wherein determining if a test compound is an uncompetitive inhibitor of a target molecule is accomplished using a high throughput screen.

41. A method comprising administering to a subject in need of inhibition of an activated target molecule a composition comprising a pathologically-activated compound identified by the method of claim 1.

42. The method of claim 41, wherein the subject is suffering from or at risk for a disease characterized by an activated target molecule.

43. The method of claim 41, wherein the activated target molecule has increased activity relative to a target molecule that is not activated.

44. The method of claim 41, wherein the activated target molecule is a target molecule that is in a different state or has different potential relative to a target molecule that is not activated.

45. The method of claim 41, wherein the activated target molecule is a target molecule expressed at a higher level than a normal or comparison level.

46. The method of claim 45, wherein each copy of the target molecule expressed at a higher level than a normal or comparison level individually has activity substantially the same as a copy of the target molecule expressed at the normal or comparison level, wherein the collective activity of the target molecule expressed at a higher level than a normal or comparison level is greater than the collective activity of the target molecule expressed at the normal or comparison level.

47. The method of claim 41, wherein the composition is produced by determining if a test compound binds uncompetitively to a target molecule, wherein if the test compound binds uncompetitively to the target molecule then the test compound is identified as a pathologically-activated targeting compound,
determining if the test compound is an uncompetitive inhibitor of the target molecule, wherein if the test compound is an uncompetitive inhibitor of the target molecule then the test compound is identified as a pathologically-activated compound, and
producing the pathologically-activated compound.

48. A method of making a pathologically-activated compound, the method comprising producing a pathologically-activated compound identified by the method of claim 1.

49. A method of forming a pathologically-activated compound, the method comprising linking a nitro group to a pathologically-activated targeting compound.

50. A method of forming a pathologically-activated compound, the method comprising linking a redox-activated group to a targeting compound, wherein the redox-activated group becomes chemically reactive when exposed to oxidizing conditions or the redox-activated group becomes chemically reactive when exposed to reducing conditions.

51. A pathologically-activated compound produced by the method of claim 36.

52. A method comprising administering to a subject in need of inhibition of a target molecule a composition comprising a pathologically-activated compound active only under conditions that occur in a pathological state.

53. The method of claim 52, wherein the conditions that occur in a pathological state are oxidizing conditions.

54. The method of claim 52, wherein the pathologically-activated compound becomes electrophilic under the conditions that occur in the pathological state.

55. A method for identifying compounds that inhibit S-nitrosylation, the method comprising comparing S-nitrosylation of a target of interest in the presence and absence of a test compound, wherein an amount of S-nitrosylation of the target measured in the presence of the test compound that is less the amount of S-nitrosylation of the target measured in the absence of the test compound identifies the test compound as an inhibitor of S-nitrosylation of the target.

56. The method of claim 55, wherein the amount of S-nitrosylation of the target is measured by detecting conversion of 2,3-diaminonaphthalene (DAN) to 2,3-naphthyltriazole (NAT).

57. The method of claim 56, wherein conversion of 2,3-diaminonaphthalene (DAN) to 2,3-naphthyltriazole (NAT) is accomplished by measuring the fluorescence of NAT.

58. The method of claim 55, wherein the target of interest is protein disulfide isomerase (PDI), dynamin-related protein 1 (Drp1), parkin, DJ-1, a MEF2 transcription factor, a matrix metalloproteinase (MMP), GAPDH, COX-2, a peroxiredoxin (Prx), or PrxII.

59. The method of claim 55, wherein nitric oxide is released in the presence of by CuCl₂.


SEQUENCE LISTING (8 sequences)

SEQ ID NO: 1 - protein, 15 aa, Homo sapiens
  MISC_FEATURE (11)..(11): modified by Sulfination
NVFVEFYAPWCGHCK

SEQ ID NO: 2 - DNA, 24 bp, Artificial Sequence
  Synthetic primer
aaacagagtagcagcgcagactgc

SEQ ID NO: 3 - DNA, 26 bp, Artificial Sequence
  Synthetic primer
ggatctctaaaactagaggcttggtg

SEQ ID NO: 4 - DNA, 21 bp, Artificial Sequence
  Synthetic primer
tctgcctttcgcctttgagac

SEQ ID NO: 5 - DNA, 21 bp, Artificial Sequence
  Synthetic primer
cccgggctgcgcactgaccac

SEQ ID NO: 6 - DNA, 21 bp, Artificial Sequence
  Synthetic primer
aaacccatcaccatcttccag

SEQ ID NO: 7 - DNA, 21 bp, Artificial Sequence
  Synthetic primer
aggggccatccacagtcttct

SEQ ID NO: 8 - protein, 4 aa, Unknown
  Thioredoxin-like domain in Mammalian PDI protein
CGHC